SEQUENCE LISTING (23 sequences)

SEQ ID NO: 1 - DNA, 243 bp, Arxula adeninivorans
gcaatattgtgcaaagatacaagcgcatttatctatagttggccagcaccaaagatcatgcctttacttctggagtctatctaggtcgttatcgattgttgcatatatgcttaaaggattgtcgaaaacagccccgtcgaggtttcgaagggggacactggcgcggcttgccagcgacgcgtattgaaagttggacatttaatagtttcggagaacagagtaattaactcttcatttggcgca

SEQ ID NO: 2 - DNA, 724 bp, Arxula adeninivorans
gaattcatatcccgggccgtcacattagcaaatccaaaaaaggcttgggtaccgagcatagggcttccagtctgattagaggatgccacctcgagacgcaggtctgctccatccaacggagcgccattatcagcagacggttcaccgcatacatttaaggtaacaaacaatgtctggttttgatggctcggaattcctgatagttcgtatctatgagtttctcctgcctgcaacgtaaaccgaatcggagtcgcctgttgtataggggtagttgtcgccgaagtgtgaaatccttagctttagttggaagcaaaaggtccacttcgatgcctttatcattgctcaatgagctttcggattctttcctaggttcccgcttccaaaagttaactgactggagattttccacaaattcaaaccctgcaacgagaaaagggtcaaggtcaaatgccagtgctaggctcggccacaccagcaccagcaatattgtgcaaagatacaagcgcatttatctatagttggccagcaccaaagatcatgccttgtacttctggagtctatctaggtcgttatcgattgttgcatatatgcttaaaggattgtcgaaaacagccccgtcgaggtttcgaagggggacactggcgcggcttgccagcgacgcgtattgaaagttggacatttaatagtttcggagaacagagtaattaactcttcatttggcgca

SEQ ID NO: 3 - DNA, 635 bp, Arxula adeninivorans
cacagaccagacggaatcatcctaacattagcaattccaaatccgccgacccgttcgttactcaggttgatcataaggggtcctcattccccaagaggagaaaacggtgtcttaattgcgcacctcttgtttttgtagcatttttcaactaactagaccgttcgttctatacctaattacaacatccgcccacctttctccttattgatttgcaattactatccgaatagcgccgatattgtttggtgccgtagatgcaatgttattacggcaggaaaaatatgtacggataattgttccgcgaaaatccggaatcgtaaattgtattatcggatctgatgaatcagctatgaaagataatatatggtatagctacgactatagtaaaccatagtcaaaatcaaaagtttcaacgttttcactgttatctcatttatccccggattggccaagaaactaaggttttttggaccctggaacatgcataatgcggagatctcactcggcccgacttggaagtgtaagggaggggatccaactcggcccgactggaaggtgcaatgagtgaatggaagtagactggaggactatataaacactcgcagcctggttacaattgacgtggacgaaacaga

SEQ ID NO: 4 - DNA, 468 bp, Arxula adeninivorans
aactctagtgccaaatagactgtacatattaccaaatactgatatccattcatctttctatctgcttatctcgagatgtacgcaatccctatctctttatctcctaggaccttgatatcattggcggaacgtaccgaagttcggttgggaatttccgagacggcgcacagtaactctatctgaaccagggacaaagattaattgaaagaaatgtctttcggctaaaaaagagtaaggaaagtgagaaagcaggcagcaaagctttgcctgggccatcggcagagcctatatttcggacatgtacgactggggctttcaattagtgtctattctaactaattttcatgtatttgcccgtcggatagccgccgggtccacccgaccccacacggcgaacatccagtcaaacggttgatataaaaagggtctaacttccgcaggtgcaggttttttcaggtcaattgtaca

SEQ ID NO: 5 - DNA, 617 bp, Arxula adeninivorans
tcctgttcactattcatctccagacatatctggtgtacctcgttgttgaatttctctcttgccgtatttaccattttgaaatctcgcagcaatttgaggagtagcaacatgccttaataggtagacctccaagctgctggacattatggtcataggatattcagtccatgtagtggcatcaagatcatgaaataatgaatcttaccactctatctgtgtagcaaaggtggcccttaaatttctcatacagtctgatcaactgtgatggtgctgtacgacgcgcaaaaaaagtaggtagaagtatcaagtcaatatttcgggcaattaagattgctcggcatttggtcctgtatattgttctccgttatgacggtaacgcctcatttatatcagatgggacctcatgaggacctgtgagacccaattatcgattatttctattccagttcggctctcgactgtcgctacttatttggagcctgttcgctgaggcgataatttaagggccaacctgtacgacccatttaggcaataattaagcttccagctattaatataaatgcctcagacgaccctttacccttttgtctataatcaactctggtcctgcaagtgag

SEQ ID NO: 6 - DNA, 680 bp, Arxula adeninivorans
gggtcctctagaaggaaagcatcttgatagagtattataatggaggagcgagcgacacctactgcaagagcgctgccatgttgataaatagcagtatcgccgggcagctttcttatatatggagactttggacatctccgataccagtaattgactttatacatcttcggaaacgaatagccgaccatgcttgccgaaggtattccacctgcaaggttttggtacaaagtatctttttggagttgcgacggtcttaaaatgcacgctagcatgtatccattttcggctactcgctttacctttttgaaatccacttctaagcctgtcattcgaggagaatggctgctagtatgtacattattgatcgtggctgttttcaaagcagaggtcgtcaatagccttgtcctttgtacaccgttgatcgcatacctcagttacctcttaatttcaccattctaacggaatccaacttgtgcacataatattcattaacagtgccacattaacatcagttcaacgctatctattttccccagctcttacagcctcttttatcatacctttggtgtggttagtaacgtgacaaaattcgacgtgccgtgcacatggcatatatcacctgataacttccacttaccaatgcccaaaaactcacagttcacccacaaaacgccaccact

SEQ ID NO: 7 - DNA, 629 bp, Arxula adeninivorans
ccttcgtacggtcggtcttcatacggtttggtcacttcagcagacactcttacaataaccttagtcttgcctaagctcacctctacatgtcctaacgactctccaaatgtaatttccacatcgcgaaattgcagtagcttgcgtccatcaacacgggtttcctgcttcaacaggtccaggacaaagtccttttcattatttgacagttcgaccttcatggtatcgagttagcatactgagcgagcactgaaaagtcaatttcgaatcacgtgagatatacgtgatcatctcatctgccactcagctgaatatatcacgtgttatacctcgtttacaacgtataaaaacacgacaaaagcgagactcaattagcagtaattgggtgattcgacagcaatgacagcgctttcgtgatcattcaattcatttcaaagtacaatcgctaccggtctcaacttctgtcatctttacgactcccatccttgatttcatctctggtcatcgcgaaaacaacgtgttgaggaaaaaaagccccaccaaccgcggcgctcgaggcgaacctcctctcgaagacacacacatatatatggaccccctgtgagtttgaaacttttacactcaactttaaa

SEQ ID NO: 8 - DNA, 515 bp, Arxula adeninivorans
gagctcaactacatttccaaagaagcattaatcgaggatatcaagactgacgttcgagtagcactcaattcgctacaacgacctgactacgaaaagtacgagaaggatgagttttggacggataattagacgtaagcctagactgtgtcttccccccacgtgtcggttgttagccggggaaacccaacatttatgacgagcatagaaatagagcataattgaatccgactgtaaagaacgaaacagaactccacatgtccatgtcctatacataacgctaacaacaattctacataatccagcgtcactatgatccaatgggctatggccccatggttaatatattgacccggtcccccccttaggaattattcaacattagtcttaaccgtatcgggtggctctggcccggcccctccggtgttatagggttgctccggtccgacgtgtatatatatgaagcccttccccggaaaccactcctcgaaaacaaaatcacatctgattaataaaaa

SEQ ID NO: 9 - DNA, 429 bp, Arxula adeninivorans
agtatattgcgatggtgtaatggcattgaatgtgtgtgctggcactgggatttgacaaaggtgtccttggacccgtggtcccccaccggccaactggacccgccaactccgtccggtatagacagaggattaaaaaataaaggaaaaagcctctcgactcctgaagcaacaaagttaactaaaaccaccatttttacaccgatttcggtttgaggcaaacgagcatccgttttacaaaaaaccagcgcagttcctagtggaggcaagcccgagacgcgcgtttgttgtaatcgattctgatcccggtggcagtcttgaaaagtgtgtgagtgagtgattattgagagtcccgtgtttgttgttgttggtcatcagtccccctttacactttcatcgcccttttattcctaataacttgaataacgtgtc

SEQ ID NO: 10 - DNA, 600 bp, Arxula adeninivorans
aagcttggcacgaaactttgcaccgtcccctacctacctcgtccctgccgggacactatgcaattgctgcagtggcctgggattggctaaccttccggggcgggtagtgacgcccgcacagctctgcacggggattgatcggtaactgatcttcatctgataatttcacaccacttcattacccaattgggacgtgcaccggtgggacccgaccgccatactcacatttaccttcggccgactggaaaaagaaagctcgcagccgggtaagggaacattttatcagattgtccataaaattgccggcaaccttccaacagcataacctttcccgagtacttatttggcgtccaaggtcctttgttatatcgacatcatcttggaaccgagtttacaactggggtgcttgcttgactttaaagtttgatcgctcacgatcgttgataagcaaagtcggttaaatttcacctagcattcatcgagggggttctttgtggttgattttatattctttcccgtcccccgtacagtaattttgctcatttgagttgttttttctgacaaagtgcttgagcatatttaccttgtgtgcataatatt

SEQ ID NO: 11 - DNA, 1009 bp, Arxula adeninivorans
aatattatgcacacaaggtaaatatgctcaagcactttgtcagaaaaaacaactcaaatgagcaaaattactgtacgggggacgggaaagaatataaaatcaaccacaaagaaccccctcgatgaatgctaggtgaaatttaaccgactttgcttatcaacgatcgtgagcgatcaaactttaaagtcaagcaagcaccccagttgtaaactcggttccaagatgatgtcgatataacaaaggaccttggacgccaaataagtactcgggaaaggttatgctgttggaaggttgccggcaattttatggacaatctgataaaatgttcccttacccggctgcgagctttctttttccagtcggccgaaggtaaatgtgagtatggcggtcgggtcccaccggtgcacgtcccaattgggtaatgaagtggtgtgaaattatcagatgaagatcagttaccgatcaatccccgtgcagagctgtgcgggcgtcactacccgccccggaaggttagccaatcccaggccactgcagcaattgcatagtgtcccggcagggacgaggtaggtaggggacggtgcaaagtttcgtgccaagctttgtgagcgccaagagagcctacagatgtcgagaccacagattttattcccgaggatttccgactaaagatcacatctgattatgccgttttgggaaggtgccgtttgcggtaaaatctgatcaacccctacacggcagttctgaaagctgagtggttgagaggtgcaggtaagggagacggcaattgggtctagaacccaacaaaaaagtaactcggagccaatctaatctgatcaaggagaaaaagtaaaaataaaccaaagcaaaaaagtaaccggctgccgaatccctttttcgggtgcgtccactgatgttggggcccgaaacggggatcgtcctggtgctgaaatataaagcaattagcaatcccaccccttaaggggacatctagacactccagcgcacatca

SEQ ID NO: 12 - DNA, 507 bp, Arxula adeninivorans
cggtatgccctggaagggtaatgctgctgccaacgatgatgacttcctcgacctcactggccagaacgcccaggcccctgacttgcctgctggtttcactgcccgaggtatcgtggctctggtcttctcctgtatttctgcattcctgggtatgggaatgatcacctggtacggactcagcgacatttccaacactgagcgaaaggtgctagagcgagaaggtgtcatttccgagggagagcaggtcgaccctattgaccccagcaccgtcgctcaccctcatcaccacgaaaattagaagagttaattgtcacgattgttttgtttgtagttaggaacatgaactggtctccttatcataataatttcgcactaaatctttggaatatcttatcttatcgggtacggctcgtccgaggagtaccgaggtaggtgactgaaaatttcacgttttgaaaggtccccaaggcagtctttcacgaaccgtgtctcccccttgacaacact

SEQ ID NO: 13 - DNA, 787 bp, Arxula adeninivorans
gtaatctctgaaaattcccaacgagaagatggtgaaagcaagcaagtagcatcccaacttagggctaccggcaatcttagtcagtaccttggtcttgtactctgtactgttagtcttgtccaataactaatgatcatcgaatccttcttatagcatacctagtctggcgacaatgttccagaagattggattgaaggcgaccgaagccaacacccctgggttagcaacttgagtcaaagtaactccacgtcgtatacgtacacttgagagagtcctgagtgaggtcaattcgagcaatttctgcaacctttgctaccgagtccattataattcagtaatcctgtaatactaaaggttcacttggcaacctgattttccaggggacgttgcttgaatgtgaccccgcggctttattggctctagggaaaattccacggtttctatatacttcttgccgccatagttcatcattctagaagctcgattagcatgacacactagaatagcacagtattctgtcctactccctctgatcaattccatcattgttaaaggcgtttgagaatttggtagactcagtaacaatgccctcacttcctgcgcgcgccgcagaagtgatcgcctatccaaacttcctaattaagacatacaatacacacgcaggatcaaagtgtccaacaggaccagaaggaataatgccaaccaattgcagcaattgaagcgaggtggggatgaaatgaggtatatatagcaaataaatatccgtttgtattgcacatatcagcta

SEQ ID NO: 14 - DNA, 688 bp, Arxula adeninivorans
cggcaaacttgtacctcacgcccaaaacggaaaagagtgtttcggatacgggccgccatgatgactccggtccggaggttgacagctcaggggcaccagatagaccttattctgaagaaaccagcacttgtagccagtaacggtcattgcagactgccatagacataggttatttgtaattttccgtctttacaatgcccattgagcaagcttttctcatccaatccagaatgatatacggtaactttcgccataatagtaccggcgatctcccagagccgcacatgagcctcttggacattcggcacaccgatcatccacctcctctcttattgccttgccatgggcgttcgatgagtaaccctcttgaagtgggtacaattcacgcctgtttgccactccgtagacgacaatagaagatcacactatccagctgtacttgacatacacgttcaccgggtcctaaactactgcctggttctccctcttttctttggacgttttcctcaacgggctcgcctacctcgcatacgatgcatgcgcctcacaatgtaaatacttgtaaatacgaaaaagaaattattgggcttcgtccccgccgatcatcgttgtgatcatggaatcaaaagtatataaactgccaaggatcccccaagcttaaaggtttcatccgcgagttagtgctgaa

SEQ ID NO: 15 - DNA, 2175 bp, Arxula adeninivorans
ttttcaatcgacgaattgcaattgacaaagagagaacatgctcttaagactgaacatcttatcagacaagggccgcatcgagcggctacctcgtaattgtggtcagtcattgacaaccattagcaagccattggcaacgggacaacgcaggcgaatctcactcgcagttactgcctgcaatgacaattgctcctcttcgcgcaaacataatagactttgataagcaccacaatcgcttcttccctcggtcgtactggcaaatttcatgcatgaatgcggctgcagcctcctgtcgatatgggacggagctctgttgctttcggggccctttccaagcttcaatccactaatcggagaatgttcatcaactggctcagctaatcagataggtcaacttgttccccagcggtttcaccacatgctgtcctttcctccccccgctcggaaggatacgcagaatgctttatcgtggggctgccccgggccgaaaccggcattttgggttgactcttttaaggaacgacatgcccaggggactctccactcacaaaccctttcaaaaaccatgagcaaaaacattatcattctgtctggagatcacgtaggacctgaggtcactgctgaggccattaaggtgctcgaagccattacccaggctcgtcctaacgtaaagttcaacttcgatcacaagctaattggaggagctgctatcgatgccaccggatctccccttcctgatgagactctggaggcttctaagaaagccgatgccgtgttgctgggagctgtaggaggtcccaagtggggaactggtgccgtccgtcctgagcagggtctgctcaagattcgaaaggaactcaacctgtatgccaacctgcgtccttgcaatttcatgtcagaaaagcttctggatctgtcccctctcaggtccgagattgtcaagggaaccaacttcactgttgttcgagagctcgtgggaggcatctattttggtacccgaaaggaagatgagggcaatggagaggcatgggacactgaaaaatacactgtggaggaggtaaagcgaattactcgaatggctgcttttctggcccttcagtctaaccctcctttgcctgtgtggagtttggacaaggctaacgtattggcttcttctcgtctgtggagaaagactgtgaccgagacaattgagaaggagttcccacagctgaccctcaaccaccagctgattgattctgcggccatgattcttatccagaacccatccaagatgaatggagtaattgtcacttcgaacatgtttggagacatcatttctgatgaggcctcagttattcctggatctctgggtctgctgccttctgcctctctgtcctctcttcccgacaagaacacggcctttggactgtacgagccttgtcacggaagtgcccccgacttgcctcctaataaggtgaaccccattgccacaattttgtctgctgccatgatgttgcgactttctttgaacctcaaggaggaagctgatgctgttgagaaggccgtcagtaaggtaattgacaatggcattgtcaccgccgatctcaagggtgcttcatctactaccgaggttggagatgccgttgctgctgaggtacagaagcttcttaaataaattcatgaatagtattaatgattggcaataaattgcaaaagtaaccaaataaagcaaaaaaaatggtaataaattgcaaaaaataggcgaagtatataggtatatataaaaaacacccacgctgggggtcgaacccagaatcttctgattagaagtcagacgcgttgccattacgccacgcaggcaaagcgataaaaaactctaggttttaggcttacaaatttggattagaaaaatcaagccaaaatcggcccgatttgggcattaacacaaaaaggactgtctaataaacagattttaagtctaagtctaaggcgttgacactgattttaactgaaactgggcgttaaaactggaagccattcacgctattatttgtaaatgtactgtccgagccaatcttcgcaaaaattgatagaattagtcgttattagatgccaatggcctatttggtaacgcctggagatagccaggagcgctaaggggtttcctcacaattaatagggactgtatctagacac

SEQ ID NO: 16 - DNA, 2175 bp, Arxula adeninivorans
ttttcaatcgacgaattgcaattgacaaagagagaacatgctcttaagactgaacatcttatcagacaagggccgcatcgagcggctacctcgtaattgtggtcagtcattgacaaccattagcaagccattggcaacgggacaacgcaggcgaatctcactcgcagttactgcctgcaatgacaattgctcctcttcgcgcaaacataatagactttgataagcaccacaatcgcttcttccctcggtcgtactggcaaatttcatgcatgaatgcggctgcagcctcctgtcgatatgggacggagctctgttgctttcggcgccctttccaagcttcaatccactaatcggagaatgttcatcaactggctcagctaatcagataggtcaacttgttccccagcggtttcaccacatgctgtcctttcctccccccgctcggaaggatacgcagaatgctttatcgtggggctgccccgggccgaaaccggcattttgggttgactcttttaaggaacgacatgcccaggggactctccactcacaaaccctttcaaaaaccatgagcaaaaacattatcattctgtctggagatcacgtaggacctgaggtcactgctgaggccattaaggtgctcgaagccattacccaggctcgtcctaacgtaaagttcaacttcgatcacaagctaattggaggagctgctatcgatgccaccggatctccccttcctgatgagactctggaggcttctaagaaagccgatgccgtgttgctgggagctgtaggaggtcccaagtggggaactggtgccgtccgtcctgagcagggtctgctcaagattcgaaaggaactcaacctgtatgccaacctgcgtccttgcaatttcatgtcagaaaagcttctggatctgtcccctctcaggtccgagattgtcaagggaaccaacttcactgttgttcgagagctcgtgggaggcatctattttggtacccgaaaggaagatgagggcaatggagaggcatgggacactgaaaaatacactgtggaggaggtaaagcgaattactcgaatggctgcttttctggcccttcagtctaaccctcctttgcctgtgtggagtttggacaaggctaacgtattggcttcttctcgtctgtggagaaagactgtgaccgagacaattgagaaggagttcccacagctgaccctcaaccaccagctgattgattctgcggccatgattcttatccagaacccatccaagatgaatggagtaattgtcacttcgaacatgtttggagacatcatttctgatgaggcctcagttattcctggatctctgggtctgctgccttctgcctctctgtcctctcttcccgacaagaacacggcctttggactgtacgagccttgtcacggaagtgcccccgacttgcctcctaataaggtgaaccccattgccacaattttgtctgctgccatgatgttgcgactttctttgaacctcaaggaggaagctgatgctgttgagaaggccgtcagtaaggtaattgacaatggcattgtcaccgccgatctcaagggtgcttcatctactaccgaggttggagatgccgttgctgctgaggtacagaagcttcttaaataaattcatgaatagtattaatgattggcaataaattgcaaaagtaaccaaataaagcaaaaaaaatggtaataaattgcaaaaaataggcgaagtatataggtatatataaaaaacacccacgctgggggtcgaacccagaatcttctgattagaagtcagacgcgttgccattacgccacgcaggcaaagcgataaaaaactctaggttttaggcttacaaatttggattagaaaaatcaagccaaaatcggcccgatttgggcattaacacaaaaaggactgtctaataaacagattttaagtctaagtctaaggcgttgacactgattttaactgaaactgggcgttaaaactggaagccattcacgctattatttgtaaatgtactgtccgagccaatcttcgcaaaaattgatagaattagtcgttattagatgccaatggcctatttggtaacgcctggagatagccaggagcgctaaggggtttcctcacaattaatagggactgtatctagacac

SEQ ID NO: 17 - DNA, 536 bp, Arxula adeninivorans
atcttctgatccgtgtgtcgctttacttttattctctctgtcgagcattactctagttggtacgactggtatgactggtatttgtggatcataactcacattagcacaggcacgtgacactggaaccgcgataaaatgatactggtaaatggacaccaagctatcaacccaatacataagactcataaaaagtagcacacatacacacacacacacccacacaacagcaatacatgtactcaacacctcacctcgcctaccgttcaccaggttgttggactgggattgcaccgagccgcacgcctgacggcaccacacggctttaaaccctcgctgcaggttccaccatggcaaaatggctcttgtcccaaagcacgcgccacacaattgggagggaaaacaacaccacaattggggcccagttttggagcaaaatatgtggccaatcacggcgctgtgccgcgccaaacctatataaaccctttgccaccctccttgtgttttttttttcttcttgataataacataaacacatc

SEQ ID NO: 18 - DNA, 281 bp, Arxula adeninivorans
ttaacttatactaacaatcaattgactatgttacaaaaatctttcctgaccgttaacggcctgtctcaccttgctcagtatttatccgaggatggttcgactgcctaatggggtaaagccggagcacggctagcatgtcatcggtatcgcccttttgggtactaactctgtggggtaaaaagcggacttgattgttccggttggattgcaaaatatttaagcattgagtttccataaagatgtgaagatttattcaatttatccattaccataatatagca

SEQ ID NO: 19 - DNA, 257 bp, Arxula adeninivorans
cctattgaccccagcaccgtcgctcaccctcatcaccacgaaaattagaagagttaattgtcacgattgttttgtttgtagttaggaacatgaactggtctccttatcataataatttcgcactaaatctttggaatatcttatcttatcgggtacggctcgtccgaggagtaccgaggtaggtgactgaaaatttcacgttttgaaaggtccccaaggcagtctttcacgaaccgtgtctcccccttgacaacact

SEQ ID NO: 20 - DNA, 1164 bp, Arxula adeninivorans
tttcgcgtcctggcggttgttttgtggtatgagcgttgatgtatttgatcggtaatcgcgtttaatcttctgatccgtgtgtcgctttacttttattctctctgtcgagcattactctagttggtacgactggtatgactggtatttgtggatcataactcacattagcacaggcacgtgacactggaaccgcgataaaatgatactggtaaatggacaccaagctatcaacccaatacataagactcataaaaagtagcacacatacacacacacacacccacacaacagcaatacatgtactcaacacctcacctcgcctaccgttcaccaggttgttggactgggattgcaccgagccgcacgcctgacggcaccacacggctttaaaccctcgctgcaggttccaccatggcaaaatggctcttgtcccaaagcacgcgccacacaattgggagggaaaacaacaccacaattggggcccagttttggagcaaaatatgtggccaatcacggcgctgtgccgcgccaaacctatataaaccctttgccaccctccttgtgttttttttttcttcttgataataacataaacacatcatgtctgggtaggtaaaaaaactgggtttgataatttttttgctgcagtactaactaaagacgcggaaaaggaggaaaaggacttggaaagggaggcgctaagcgtcaccgaaagattctacgtgacaacattcagggtattaccaagcccgccatccgtcgtcttgctcgacgaggaggtgtcaagcgtatctcggccctcatttatgaggagacccgatcggtgctcaagacctttttggagtctgtgatccgagatgccgtcacctacactgagcacgccaagagaaagactgtgacttctcttgatgtcgtgtacgctctcaagcgacagggacgaactctttatggttttggaggttaatttttttatttaaggataatttatgggtttaacactacaacacttagcatacactagagcactgaacgtatccttgaactcgtaacctatactagaacacgtagacatatatttgccacttgccacctccgtgacccctggactgccggttccatttgaccgtagccgccactgcaatgtcgtggtagcatttggtgatg

SEQ ID NO: 21 - protein, 103 aa, Arxula adeninivorans
MSGRGKGGKGLGKGGAKRHRKILRDNIQGITKPAIRRLARRGGVKRISALIYEETRSVLKTFLESVIRDAVTYTEHAKRKTVTSLDVVYALKRQGRTLYGFGG

SEQ ID NO: 22 - DNA, 1811 bp, Arxula adeninivorans
ctgtgagtggatctgcgtagacgataagatgaatgtttgggaaaccagagaacaggggcgtcccaatgtcgaagaaatgacaataagtttcgctggtaatgggccatgctgggttgcagcgtgcggaccagatccattgctacgggcggtgcaaaatggtgtggcaaagctcatctccgatggccacgacgtgcatcttcatattgaggcctttggctggtaattaacttatactaacaatcaattgactatgttacaaaaatctttcctgaccgttaacggcctgtctcaccttgctcagtatttatccgaggatggttcgactgcctaatggggtaaagccggagcacggctagcatgtcatcggtatcgcccttttgggtactaactctgtggggtaaaaagcggacttgattgttccggttggattgcaaaatatttaagcattgagtttccataaagatgtgaagatttattcaatttatccattaccataatatagcaatggctgcacaagtcgaggaacaggtgcttaatctccgagctcaagcggatcacaatccctcgtttgttctcaaaaagcccttggaattggggtttgaagagcgcccagtgcctgtgattactgaccctcgagatgttaaaatccaggtcaagaagacgggaatctgcggatctgatgttcacttttggcaacatggtcgtattggtgactacgtagtggaaaagccaatggtcctaggccatgagtccagtggagtggtggttgaagttggttctgaggtcacctccctcaaggtgggagaccgagtagctatggaacctggagtgccggacaggcgctcaaaggaatataagatgggacgatatcatttgtgccctcatgtgcgctttgcagcctgccctcccactgacggaacgctctgcaagtattacacacttcccgaggacttctgcgttaaactgcctgaaaacgtagattttgaggaaggagcattggtggagccattgagtgtagcagtccacaccgctaggcttcttggtatctaccctggctccaaggtggtcgtgttcggagcaggaccaattggccagctgtgcattggagtgtgcaaggcatttggtgccagtataattggcgcggtggatttgtttgagcaaaagctagagaccgcaaaggagtttggtgcatctcacacgtatgtccctcagaaaggggactcacatgacgagactgcccacaaaattctcgagctcctgcccaacaagcaggccccagatgtggtcattgatgccagtggagctgagcaatctatcaacgcaggaattgagcttctagagagaggtggaacctttggacaggtggccatgggccgaactgactacattcagtttgccgtttctcgtatggccatgaaggagattcgattccagggagtcttccgatatacctatggagattatgagctcgcaacccagttgattggtgatggaaagatccctgtaaagaaattggtcacccataggcggccatttgaaaaggccgaggaggcatatgaattggtcaagagcggtgttgcagtatataattgatgcctacgattcattgttttcggacacagttagttgttttccttttcccatctattcacattgttgaactgctattcataataacactatattggtcgtagtcccgtactgttgaatacaggatctcttcattccctcagcgggcatttaagggcactttgatatggccttaggttgggcttcttgggctctctcgcgatctcgcctagcgaagatct

SEQ ID NO: 23 - protein, 368 aa, Arxula adeninivorans
MAAQVEEQVLNLRAQADHNPSFVLKKPLELGFEERPVPVITDPRDVKIQVKKTGICGSDVHFWQHGRIGDYVVEKPMVLGHESSGVVVEVGSEVTSLKVGDRVAMEPGVPDRRSKEYKMGRYHLCPHVRFAACPPTDGTLCKYYTLPEDFCVKLPENVDFEEGALVEPLSVAVHTARLLGIYPGSKVVVFGAGPIGQLCIGVCKAFGASIIGAVDLFEQKLETAKEFGASHTYVPQKGDSHDETAHKILELLPNKQAPDVVIDASGAEQSINAGIELLERGGTFGQVAMGRTDYIQFAVSRMAMKEIRFQGVFRYTYGDYELATQLIGDGKIPVKKLVTHRRPFEKAEEAYELVKSGVAVKCIIDGPE

(19) United States
(12) Patent Application Publication
(10) Pub. No.: US 2003/0186376 A1
(43) Pub. Date: Oct. 2, 2003

(54) Method for synthesizing proteins in a yeast of the genus Arxula and suitable promoters

(75) Inventors: Gotthard Kunze, Gatersleben (DE); Ines Walter, Egloffstein (DE); Harald Rosel, Leipzig (DE); Erik Boer, Langenstein (DE); Regina Stoltenburg, Leipzig (DE); Thomas Wartmann, Aderstedt (DE); Bui Minh Duc, Wien (AT)

Correspondence Address: MORGAN, LEWIS & BOCKIUS LLP, 1701 MARKET STREET, PHILADELPHIA, PA, 19103-2921, US

(73) Assignee: Rhein Biotech Gesellschaft Fur Neue Biotechnologische Prozesse Und Produkte MBH

(21) Appl. No.: 10/291,307

(22) Filed: Nov. 8, 2002

Related U.S. Application Data
(63) Continuation of application No. PCT/EP01/05225, filed on May 8, 2001.

(30) Foreign Application Priority Data
May 8, 2000 (DE) 100 22 334.6

(51) Int. Cl. C12P 21/02; C12N 1/18; C12N 15/74

(52) U.S. Cl. 435/69.1; 435/254.2; 435/320.1

(57) ABSTRACT

The invention relates to a method for synthesising one protein or several proteins in a host cell of the yeast genus Arxula and a nucleic acid molecule including a suitable promoter for this purpose. Moreover, this invention relates to an expression vector, a host cell and a kit as well as the use of these. Object of the invention is to make available a method for synthesising a protein in a yeast of the genus Arxula, with which higher protein quantities can be obtained. This object is solved by a method for synthesising one protein or several proteins in a host cell of the yeast genus Arxula, comprising of (i) cloning of at least one nucleic acid, which encodes a heterologous protein, in an expression vector that contains an inducible promoter from a yeast of the Arxula genus or a promoter from an constitutive expressed gene, selected from the ARFC3 gene and the AHSB4 gene, from a yeast of the Arxula genus, so that the cloned nucleic acid is under the transcriptional control of the promoter; (ii) insertion of the expression vector obtained into a host cell of the yeast genus Arxula suitable for synthesizing a heterologous protein; (iii) cultivating of host cell obtained in (ii) and, if necessary, (iv) inducing of the promoter; (v) harvesting of the protein in a known manner.

### CROSS-REFERENCE TO RELATED APPLICATIONS

This application is a continuation of PCT Application No. PCT/EP01/05225, filed May 8, 2001, which claims the benefit of priority to German Patent Application No. 10022334.6, filed May 8, 2000.

### BACKGROUND OF THE INVENTION

This invention relates to a method for synthesising one or several proteins in a host cell of the yeast genus Arxula and a nucleic acid molecule, including a suitable promoter for this purpose. Moreover, this invention relates to an expression vector, a host cell and a kit as well as use of these.

Yeasts have been used since the eighties as producers of heterologous proteins. For example, e.g. HbsAg, α1-antitrypsin, hirudin, glucoamylase, EBV Envelope Protein, h-SOD, HSA, h-interferon α and h-EGF have been synthesized in S. cerevisiaeas heterologous proteins (Gellissen & Hollenberg 1997). There has been success with heterologous gene expression. But, genes, which especially code secretory proteins, usually cannot be expressed satisfactory in S. cerevisiase. Thus, it was started early on establishing host-vector systems with non-conventional yeast, such as Kluyveromyces lactis, Hansenula polymorpha and Pichia pastoris. As a result, higher yields of heterologous proteins have been achieved. For example, prochymosin could be produced in K. lactis in a magnitude of a few g/l. However, these systems do not meet all requirements placed on them. For example, problems arise, especially when heterologous secretory proteins are synthesised, which restrict the use of yeast for heterologous gene expression (Sudbery 1996, Hollenberg & Gellissen 1997, Gellissen & Hollenberg 1997).

For expression of heterologous genes, up to now, yeast species were used, which form yeast cells or pseudomycelium. Since filamentous fungi, such as Aspergillus species, are known as much better “secreter,” especially for glycoproteins, the use of yeast that can also form myceliums is a possibility for improving heterologous gene expression. Unfortunately, most mycelium-forming yeast species are pathogenic (e.g. Candida albicans) or have hardly been investigated. A non-pathogenic yeast, which has been investigated genetically and molecular biologically and which can form mycelium under certain cultivation conditions is Arxula.

This yeast genus, with its most well known species A. adeninivorans, was first described by Middelhoven et al. in 1984. First, they called this dimorphic, xerotolerant, ascomycota, anamorphous, arthroconidal and nitrate-positive yeast Trichosporon adeninovorans, before von der Walt changed the name to A. adeninivoransin 1990. This species is characterised by extraordinary biochemical properties. Underlines are, e.g. the large spectrum of substances (among others, adenine and other purines), which can be used as C or N source, resistance to NaCl (growth up to a NaCl concentration of 17.5%) and the extraordinary high thermotolerance for yeast. The maximum growth speed in sugar-containing medium is achieved at temperatures between 37° C. and 41° C. However, also at temperatures above 45° C. A. adeninivoranscan still be cultivated, without the necessity of prior adaptation to this extremely high growth temperature.

In order to be able to use yeast of the genus Arxula and especially A. adeninivorans for synthesising proteins, transcription promoters must be isolated, with which strong expression of heterologous genes can be achieved. Up to now, however, only promoters have been used, e.g. AILV1-promoter, leading to a very weak expression of heterologous genes (for example XylE gene of Pseudomonas putida) in Arxula, and thus are not suitable for large-scale protein production. Thus, there is a need for an efficient and regulatable method for expressing proteins in Arxula and a concomitant need for promoters suitable for use in such protein expression in Arxula.

### BRIEF SUMMARY OF THE INVENTION

The invention relates to a method for synthesising one protein or several proteins in a host cell of the yeast genus Arxula and a nucleic acid molecule including a suitable promoter for this purpose. Moreover, this invention relates to an expression vector, a host cell and a kit as well as the use of these. Object of the invention is to make available a method for synthesising a protein in a yeast of the genus Arxula, with which higher protein quantities can be obtained. This object is solved by a method for synthesising one protein or several proteins in a host cell of the yeast genus Arxula, comprising of (i) cloning of at least one nucleic acid, which encodes a heterologous protein, in an expression vector that contains an inducible promoter from a yeast of the Arxula genus or a promoter from an constitutive expressed gene, selected from the ARFC3 gene and the AHSB4 gene, from a yeast of the Arxula genus, so that the cloned nucleic acid is under the transcriptional control of the promoter; (ii) insertion of the expression vector obtained into a host cell of the yeast genus Arxula suitable for synthesizing a heterologous protein; (iii) cultivating of host cell obtained in (ii) and, if necessary, (iv) inducing of the promoter; (v) harvesting of the protein in a known manner.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 illustrates the nucleotide sequence of the ARFC3 promoter from A. adeninivorans (SEQ ID NO: 1). The TATA-box and MluI-location have been double or single underlined. The arrow points to the transcription start point.

FIG. 2 illustrates the ARFC3 transcription concentration in (A-C) A. adeninivorans LS3-30° C. For this purpose, the cultures were cultivated in YMM, without (A) and with 5% (B) or 10% NaCl addition (C), the cells were harvested after (1) 20, (2) 36, (3) 45, (4) 60 and (5) 70-hour cultivation, the total RNA was isolated and used for Northern hybridisation. A BglII/Dral DNA fragment with a part of the ARFC3 gene from A. adeninivorans was used as radioactively labelled probe.

FIG. 3 illustrates the gene and restriction map of 3323 bp size DNA fragment with the AHSB4 gene.

FIG. 4 illustrates the nucleotide sequence (including promoter and terminator region) (SEQ ID NO: 20) and the derived amino acid sequence (SEQ ID NO: 21) of the AHSB4 gene.

FIG. 5 illustrates the AHSB4 transcript analysis of A. adeninivoransLS3. For this purpose, the yeast cells were cultivated for A) for 20, 36, 40, 60 and 70-h (1-5) in YMM with 2% maltose at 30° C. or (B) after 45-hour cultivation in YMM with 2% Maltose, the cells were changed in YMM with 2% maltose and 5% NaCl and cultivated for another 0, 0.5, 1, 2, and 19 h (1-5) at 30° C. Then, RNA was isolated from the cells and the B4 transcript concentration was determined by means of Northern hybridisation. A 0.8 kbp EcoRI/XhoI DNA fragment with the AHSB4 gene was used as radioactively labelled probe.

FIG. 6 illustrates the nucleotide sequence of the GAA promoter of A. adeninivorans (SEQ ID NO: 3). The TATA box and the CAAT box have been double and simple underlined. The arrows point to the transcription start points.

FIG. 7 illustrates the GAA transcript concentration in (A) A. adeninivorans LS3-30° C. (yeast cell culture), (B) LS3-45° C. (mycelium culture), (C) 135-30° C. (mycelium culture). For this purpose, the cultures were cultivated in YMM with 1% maltose as C source. The cells were harvested after (1) 20, (2) 36, (3) 45, (4) 60 and (5) 70 hours of cultivation. The total RNA was isolated and used for Northern hybridisation. A 1.6 kbp BamHI/EcoRI-DNA fragment with the GAA gene of A. adeninivorans was used as radioactively labelled probe.

FIG. 8 illustrates the nucleotide sequence of the AACP1O promoter from A. adeninivorans (SEQ ID NO: 4). The TATA box and CAAT box have been double and single underlined.

FIG. 9 illustrates the AACP 10 transcript concentration in (A) A. adeninivorans LS3-30° C. (yeast cell culture), (B) LS3-45° C. (mycelium culture), (C) 135-30° C. (mycelium culture). For this purpose, the cultures were cultivated in YMM with 1% maltose as C source. The cells were harvested after (1) 20, (2) 36, (3) 45, (4) 60 and (5) 70 hours of cultivation. The total RNA was isolated and used for Northern hybridisation. A 1.5 kbp HindIII/SalI-DNA-fragment with the AACP 10 gene from A. adeninivorans was used as radioactively labelled probe.

FIG. 10 illustrates the nucleotide sequence of the APCR17 promoter of A. adeninivorans (SEQ ID NO: 5). The TATA box was double underlined.

FIG. 11 illustrates the APCR17 transcript concentration in A. adeninivorans LS3-30° C. (yeast cell culture) [1, 3] und LS3-45° C. (mycelium culture) [2, 4]. For this purpose, the cultures were cultivated in YMM with 1% maltose as C source. The cells were harvested after 40-hour cultivation. The total RNA was isolated and used for Northern hybridisation. A 0.3 kbp EcoRI/HindIII-DNA fragment of APCR1 gene from A. adeninivorans was used as radioactively labelled probe.

FIG. 11 illustrates the APCR17 transcript concentration in A. adeninivorans LS3-30° C. (yeast cell culture) [1, 3] und LS3-45° C. (mycelium culture) [2, 4]. For this purpose, the cultures were cultivated in YMM with 1% maltose as C source. The cells were harvested after 40-hour cultivation. The total RNA was isolated and used for Northern hybridisation. A 0.3 kbp EcoRI/HindIII-DNA fragment of APCR1 gene from A. adeninivorans was used as radioactively labelled probe.

FIG. 12 illustrates the gene and restriction map of a 3451 bp size DNA fragment of the chromosomal DNA of A. adeninivorans LS3 with the APRE4- and APCR17 promoters and the associated genes.

FIG. 13 illustrates the nucleotide sequence of APRE4 promoter of A. adeninivorans (SEQ ID NO: 6). Die TATA box has been double underlined.

FIG. 14 illustrates the APRE4 transcript concentration in (A) A. adeninivorans LS3-30° C. (yeast cell culture), (B) LS3-45° C. (mycelium culture), (D) 135-30° C. (mycelium culture). For this purpose, cultures are cultivated in YMM with 1% maltose as C source. The cells are harvested after (1) 20, (2) 35, (3) 45, (4) 60 and (5) 70 hours of cultivation. The total RNA is isolated and used for Northern hybridisation. A 0.8 kbp ApaI/HindIII-DNA fragment with the APRE4 gene from A. adeninivorans is used as radioactively labelled probe.

FIG. 15 illustrates the nucleotide sequence of the AACE1 promoter from A. adeninivorans (SEQ ID NO: 7). The two potential TATA boxes have been double underlined.

FIG. 16 illustrates the AACE1 transcript concentration in A. adeninivorans LS3-30° C. For this, the cultures were cultivated for 40 hours in YMM with 1% maltose as C source. Then, transferred into a salt-containing medium (5% NaCl -end concentration) and the cells were harvested after 0 (1), 0.5 (2), 1 (3), 2 (4) or 4-hour (5) cultivation. The total RNA was isolated thereof and used for Northern hybridisation. A 1.0 kbp HindIII/XhoI-DNA-fragment with the AACE1 from A. adeninivorans was used as radioactively labelled probe.

FIG. 17 illustrates the nucleotide sequence of the ATAL1 promoter from A. adeninivorans (SEQ ID NO: 8). The TATA box and the CAAT box have been underlined double and single.

FIG. 18 illustrates the ATAL1 transcript concentration in A. adeninivorans LS3-30° C. For this, cultures were cultivated for 40 hours in YMM with 1% maltose as C source and subsequently transferred to a salt-containing medium (5% NaCl end concentration) and the cells were harvested after 0 (1), 0.5 (2), 1(3) 2 (4) or 4 (5) hours of cultivation. The total RNA was isolated and used for Northern hybridisation. A 0.3 kbp EcoRI-cDNA fragment with the ATAL1 gene from A. adeninivorans was used as radioactively labelled probe.

FIG. 19 illustrates the nucleotide sequence of the AGLC7 promoter of A. adeninivorans (SEQ ID NO: 9). The TATA box has been double underlined.

FIG. 20 illustrates the AGLC7 transcript concentration in A. adeninivorans LS3-30° C. For this, cultures were cultivated for 40 hours in YMM with 1% maltose as C source, then transferred into a salt-containing medium (5% NaCl end concentration) and the cells were harvested after 0 (1), 0.5 (20, 1 (3), 2 (4) or 4-hour (5) cultivation. The total RNA was isolated and used for Northern hybridisation. A 0.4 kbp EcoRI/XbaI-DNA fragment with the AGLC7 gene from A. adeninivorans was used as radioactively labelled probe.

FIG. 21 illustrates the nucleotide sequence of the AFET3 promoter from A. adeninivorans (SEQ ID NO: 10). Die TATA box has been double underlined.

FIG. 22 illustrates the AFET3 transcript concentration in (A) A. adeninivorans LS3-30° C. (yeast cell culture), (B) LS3-45° C. (mycelium culture), (C) 135-30° C. (mycelium culture). For this, the cultures were cultivated in YMM with 1% maltose as C source without (1) and with 1 (2), 5 (3), 10 (4), 50 (5) and 200 μM FeEDTA (6), the cells were cultivated after 20-hour cultivation. The total RNA was isolated thereof and used for Northern hybridisation. A 1.2 kbp HindIII/SalI-DNA fragment with the AFET3 gene from A. adeninivorans was used as radioactively labelled probe.

FIG. 23 illustrates the gene and restriction map of the 3451 bp size DNA fragment with the AFET3 and AFTR1 promoters and the associated genes.

FIG. 24 illustrates the nucleotide sequence of the AFTR1 promoter from A. adeninivorans (SEQ ID NO: 11). The TATA box and CAAT box has been double and single underlined.

FIG. 25 illustrates the nucleotide sequence of the AEFG1 promoter from A. adeninivorans (SEQ ID NO: 12).

FIG. 26 illustrates the AEFG1 transcript concentration in (A) A. adeninivorans LS3-30° C. (yeast cell culture), (B) LS3-45° C. (mycelium culture), (C) 135-30° C. (mycelium culture) after a shift from aerobic to anaerobic cultivation conditions. For this, the cells were cultivated for 40 hours under aerobic conditions in YMM with 1% maltose as C source (1), and then incubated under anaerobic conditions for another 2, 4, 6, 9, and 24 hours (2-6), the total RNA was isolated and used for Northern hybridisations. A 2.1 kbp HindIII-DNA fragment with the AEFG1 gene from A. adeninivorans was used as radioactively labelled probe.

FIG. 27 illustrates the nucleotide sequence of the ASTE6 promoter from A. adeninivorans (SEQ ID NO: 13). The three potential TATA boxes have been underlined.

FIG. 28 illustrates the nucleotide sequence of the APMD 1 promoter of A. adeninivorans (SEQ ID NO: 14). The three potential TATA boxes have been underlined.

FIG. 29 illustrates a diagram with the degradation metabolism of D-xylose to D-xylulose-5P.

FIG. 30 illustrates the gene and restriction map of the 2418 bp sized DNA fragment with the AXDH gene.

FIG. 31 illustrates the nucleotide sequence (including promoter and terminator region) (SEQ ID NO: 22) and the derived amino acid sequence (SEQ ID NO: 23) of the AXDH gene.

FIG. 32 illustrates the AXDH transcript analysis of A. adeninivorans LS3. For this, yeast cells were cultivated for 24 h (1, 3, 5, 7, 9, 11, 13, 15) or 48 h (2, 4, 6, 8, 10, 12, 14, 16) in YMM with 2% glucose (1, 2), 2% galactose (3, 4), 2% sorbite (5, 6), 2% fructose (7, 8), 2% maltose (9, 10), 2% mannite (11, 12), 2% xylit (13, 14) and 2% xylose (15, 16). Then the RNA was isolated from the cells and the AXDH transcript concentration was determined by means of Northern hybridisation. A 0.7 kbp XhoI-DNA fragment with the AXDH gene was used as radioactively labelled probe.

FIG. 33 illustrates proof of the inducibility of the AXDH promoter from A. adeninivorans LS3. For this, the yeast cells were cultivated in YMM with 12% gluclose for 48 hours and then transferred to YMM with (A,B) 2% xylit, (C) 2% galactose or (D) 5% sorbite. From these cultures, the cells were harvested after 0 (1), 0.5 (2), 1 (3), 2 (4) and 19 h (5), and the total RNA isolated, which is used for Northern hybridisation. The DNA with the AXDH gene (B-D) or with the TEF1 gene (A) was used as radioactively labelled probe.

FIG. 34 illustrates the influence of the xylit concentration on the AXDH transcript accumulation. For this, the yeast cells were cultivated for 48 hours in YMM with 2% Glucose and then transferred to YMM with (A) 2%, (B) 5% and (C) 23% xylit. From these cultures, the cells were harvested after 0 (1), 0.5 (2), 1 (3), 2 (4) and 19 h (5), the RNA isolated that is used for Northern hybridization. The DNA fragment with the AXDH gene was used as radioactively labelled probe.

FIG. 35 illustrates the influence of xylit concentration on the growth capacity of A. adeninivorans LS3. For this, yeast cells were cultivated in YMM with 2%, 5% or 23% xylit at 30° C. and the optical density at 600 nm was measured during growth.

FIG. 36 illustrates the gene and restriction map of the 2838 bp size DNA fragment with the ALEU2 gene.

FIG. 37 illustrates the gene and restriction map of the 2185 bp size DNA fragment with the ALEU2m gene.

FIG. 38 illustrates the construction schema for the plasmid pAL-ALEU2m.

FIG. 39 illustrates the results of a Southern hybridisation for determining the plasmid copy number integrated into the genome of A. adeninivorans G1211 /pAL-ALEU2m. Chromosomal DNA from (1) G1211/pAL-ALEU2m-1, (2) G1211/pAL-ALEU2m-2, (3) G1211/pAL-ALEU2m-3, (4) G1211/pAL-ALEU2m-4, (5) G1211/pAL-ALEU2m-5, (6) G1211/pAL-ALEU2m-6 and G1211 (7) was constrained with BglII and hybridised against the radioactively marked plasmid (A) pUC19 (cleaved with BamHI) or against a BglII-DNA fragment with the ALEU2m gene.

FIG. 40 illustrates the construction schema for the plasmid pBS-ALEU2-SwARS1.

FIG. 41 illustrates the construction schema for the plasmid pAL-HPH-AXDH(ATG)-GFP.

FIG. 42 illustrates the proof of the GFP in A. adeninivorans LS3/pAL-HPH-AXDH(ATG)-GFP. For this, the cultures were cultivated for 48 hours in YMM with 2% xylit and then the transmission and the GFP fluorescence of the cells was analysed with the help of the fluorescence microscope.

FIG. 43 illustrates the proof of the GFP in A. adeninivorans 135/pAL-HPH-AXDH(ATG)-GFP. For this, the cultures were cultivated for 48 h in YMM with 2% xylit and then the transmission and the GFP fluorescence of the cells was analysed with the help of the fluorescence microscope.

FIG. 44 illustrates the results of a Western blot analysis for identification of the GFP in protein extracts of A. adeninivorans LS3 (1), LS3/pAL-HPH-AXDH(ATG)-GFP1/1-93-42-7 (2), LS3/pAL-HPH-AXDH(ATG)-GFP1/1-93-42-9 (3), LS3/pAL-HPH-AXDH(ATG)-GFP17-106-132-12 (4) and LS3/pAL-HPH-AXDH(ATG)-GFP17-106-132-15 (5). An anti-GFP antibody (Molecular Probes, USA) was used as antibody.

FIG. 45 illustrates the results of a Western blot analysis for identification of the GFP in protein extracts of A. adeninivorans 135 (1), 135/pAL-HPH-AXDH(ATG)-GFP17-106-132-1 (2), 135/pAL-HPH-AXDH(ATG)-GFP17-93-117-4 (3), 135/pAL-HPH-AXDH(ATG)-GFP17-106-132-17 (4) and 135/pAL-HPH-AXDH(ATG)-GFP17-106-132-18 (5). An anti-GFP antibody (Molecular Probes, USA) was used as antibody.

FIG. 46 illustrates the construction schema for the plasmid pAL-HPH-AXDH-HSA.

FIG. 47 illustrates the results of a Western blot analysis for identification of the HSA in the cultivation medium in A. adeninivorans 135 (1), 135/pAL-HPH-AXDH-HSA-8-129 (2) and 135/pAL-HPH-AXDH-HSA-8-130. An anti HSA antibody (Biotrend Chemikalien GmbH, BRD) was used as antibody.

FIG. 48 illustrates the construction schema for the plasmid pAL-HPH-AHSB4(ATG)-GFP.

FIG. 49 illustrates the construction schema for the plasmid pAL-HPH-AHSB4-HSA.

FIG. 50 illustrates the gene and restriction map for the transformation of the linearised plasmid pAL-HPH-lacZ used in A. adeninivorans.

FIG. 51 illustrates the lacZ transcript concentration in A. adeninivorans LS3/pAL-HPH1-lacZ-80, cultivated (A) at 30° C. (yeast cells) or (B) at 45° C. (myceliums) and 135/pAL-HPH1-lacZ-2, cultivated (C) at 30° C. (mycelium). For this, the cultures were cultivated in YMM with maltose as C source. The cells were harvested after (1) 20, (2) 36, (3) 45, (4) 60 and (5) 70-hour cultivation. The total RNA was isolated thereof and used for Northern hybridisation. A 3 kbp BamHI/EcoRI-DNA fragment with the IacZ gene was used as radioactively labelled probe.

FIG. 52 illustrates the TEF1-, GAA- and lacZ transcript concentration in A. adeninivorans LS3 cultivated at 30° C. (yeast cells) and LS3/pAL-HPH-GAA-lacZ-80/1 cultivated at 30° C. (yeast cells) or 45° C. (mycelium). For this, the cultures were cultivated in YMM with maltose as C source. The cells were harvested after 20, 36, 45, 60 and 70-hour cultivation. The RNA was isolated and used for Northern hybridisation. A 1.6 kbp BamHI/EcoRI-DNA fragment with the GAA-gene or a 3 kbp BamHI/EcoRI-DNA fragment with the lacZ gene was used as radioactively labelled probe.

FIG. 53 illustrates the results of a Western blot analysis for identification of the intracellular analysis of β-galactosidase (lacZp) and the extracellular glucoamylase (Gaap) of A. adeninivorans LS3e/pAL-HPH-GAA-lacZ-80/cultivated at 30° C. (yeast cells) or 45° C. (mycelium), 135 /pAL-HPH-GAA-lacZ-2 cultivated at 30° C. (mycelium) and LS3 cultivated at 30° C. (yeast cells). An anti-Gaap antibody and an anti-B-galactosidase antibody (Boehringer-Mannheim, BRD) was used as antibody.

FIG. 54 illustrates the construction scheme for the plasmid pBS-ARFC3-PHO5-EBN.

FIG. 55 illustrates the construction scheme for the plasmid pAL-HPH-ARFC3-HSA.

FIG. 56 illustrates the construction scheme for the plasmid pAL-HPH-AEFG1 (ATG)-GFP.

FIG. 57 illustrates the construction scheme for the plasmid pAL-HPH-AEFG1-HSA.

FIG. 58 illustrates the results of a Western blot analysis for identification of the HSA in the cultivation medium of A. adeninivorans LS3 (1), LS3/pAL-HPH-AEFG1-HSA-32-124 (2), 135/pAL-HPH-AEFG1-HSA-32-125 (3) and 135 (4). An anti-HAS antibody (Biotrend Chemikalien GmbH, BRD) was used as antibody.

### DETAILED DESCRIPTION OF THE INVENTION

In order to be able to use yeast of the genus Arxula and especially A. adeninivorans for synthesising proteins, transcription promoters, with which strong expression of heterologous genes can be achieved, must be isolated. Until the present invention, however, only promoters have been used in Arxula, e.g. AILV1 promoter, leading to a very weak expression of heterologous genes (for example, XylE gene of Pseudomonas putida), and thus are not suitable for large-scale protein production. Thus, there is a need for an efficient and regulatable method for expressing proteins in Arxula and a concomitant need for promoters suitable for use in such protein expression in Arxula.

It is an object of the present invention to make available a method for synthesizing proteins in a yeast of the genus Arxula, with which method high protein yields can be obtained. This particular object is achieved by a method for synthesising one or several proteins in a host cell of the yeast genus Arxula, consisting of:

(i) cloning of at least a nucleic acid, which encodes a heterologous protein, in an expression vector, containing a inducible promoter from a yeast of the genus Arxula

or a promoter from a constitutively expressed gene, selected from the ARFC3 gene and the AHSB4 gene, from a yeast of the genus Arxula, so that the cloned nucleic acid is under the transcriptional control of the promoter;

(ii) insertion of the expression vector obtained in (i) into a host cell of the yeast genus Arxula, suitable for synthesizing a heterologous protein;

(iii) cultivating the host cell obtained in (ii), and if necessary

(iv) inducing the promoter;

(v) harvesting of protein in a known way.

An “inducible promoter” according to the invention is a nucleic acid sequence, which under the respective induction conditions, for example, higher temperature or salt concentration or anaerobic conditions, leads to at least a double initiation frequency of the transcription of a coding nucleic acid for a heterologous protein, which is under its control in comparison with non-induction conditions. The transcription frequency can be indirectly determined on the basis of the concentration of the respective transcript, measured by Northern hybridisation. A “constitutive promoter” according to the invention is a nucleic acid sequence, which causes basically the same initiation frequency under different conditions. According to the invention, a “nucleic acid, which encodes a heterologous protein” is a coding nucleic acid sequence, derived from another gene than the promoter used as control of its transcription.

In an embodiment of the method according to the invention, the promoter derived from the ARFC3 gene of the genus Arxula comprises the sequence given in SEQ ID NO: 1 or SEQ ID NO: 2. In another embodiment, the AHSB4 promoter derived from the AHSB4 gene of the yeast of the genus Arxula comprises the sequence given in SEQ ID NO: 17.

In another embodiment of the method in this invention, the inducible promoter is derived from a gene of Arxula yeast, whose expression is associated with a change in cell morphology. Preferably, the promoter is derived from the GAA gene, the AACP10gene, the PCR17 gene, or the APRE4 gene of a yeast of the genus Arxula. Especially preferred promoters comprise at least one of the sequences given in SEQ ID NO: 3, SEQ ID NO: 4, SEQ ID NO: 5 or SEQ ID NO: 6.

In another embodiment of the method according to the invention, the promoter is inducible by changing the salt concentration in culture medium. Preferred promoters of this type come from the AACE1 gene, ATAL1 gene or AGLC7 gene of a yeast of the genus Arxula. Especially preferred the promoter comprises the sequence given in SEQ ID NO: 7, SEQ ID NO: 8 or SEQ ID NO: 9.

In another embodiment of the method according to the invention, the promoter is inducible by changing the salt and/or Fe(II) ion concentration in culture medium. Preferred promoters of this type come from the AFET3 gene or the AFTR1 gene of a yeast of the genus Arxula. Preferably, the promoter comprises the sequence given in SEQ ID NO: 10 or SEQ ID NO: 11.

A promoter, which is inducible by changing the salt and/or oxygen concentration in culture medium, is used in another embodiment of the method according to the invention. Preferably, such a promoter is derived from the AEFG1 gene of a yeast of the genus Arxula. Especially preferred is a promoter, which comprises the sequence given in SEQ ID NO: 19.

In another embodiment of the method according to the invention, the promoter is inducible by a toxic agent. Examples for toxic agents, which can induce Arxula promoters are, among others, leptomycin B, valinomycin, chloroquine, cycloheximide, staurosporin and actinomycin D. Concentrations of the toxic agent suitable for induction of promoters according to the invention are ±0.008 μg/ml. The invention makes available a promoter, which is inducible by a toxic agent, for example by cycloheximide or chloramphenicol, derived from the ASTE6 gene and preferably including the sequence given in SEQ ID NO: 13. A promoter inducible by leptomycin B, derived from the APMD 1 gene of a yeast of the genus Arxula and preferably including the sequence given in SEQ ID NO: 14 is especially preferred.

In another embodiment of the method according to the invention the inducible promoters can be induced by xylit, xylolse, sorbite, and/or galactose. A promoter, derived from the AXDH gene of a yeast of the genus Arxula and preferably comprising the sequence given in SEQ ID NO: 18 is especially preferred.

Promoters derived from a gene of the yeast Arxula adeninivorans are preferred for the implementation of the method according to the invention.

The expression vector used in the method according to the invention can contain at least a marker gene. Preferably the marker gene is the ALEU2 gene of Arxula adeninivorans or a variant thereof, which retains the function of ALEU2 gene (e.g. can complement leu2-auxotrophy). Especially preferred is a marker gene with the sequence given in SEQ ID NO: 15 or SEQ ID NO: 16. Other marker genes, e.g. the LYS2 gene of S. cerevisiaeor A. adeninivorans, or the AILV1 gene of A. adeninivorans, can also be used.

The expression factor can comprise a sequence, which makes possible the integration of the vector or part of it into the genome of the host cell, for example, a sequence from the 25S-rDNA of yeast of the genus Arxula, especially A. adeninivorans . Alternatively the expression vector can comprise sequences, which make possible the replication of the vector in the host cell, such as ARS-sequences from S. cerevisiae , Schwanniomyces occidentalis, Hansenula polymorpha, Arxula adeninivorans, Yarrowia lipolytica, Picha pastoris, Kluyveromyces lactis or Debaryomyces hansenii.

The invention also makes available a nucleic acid molecule, which is suitable as promoters for expression of heterologous genes for synthesising one or several proteins in a host cell of the yeast genus Arxula.

In an embodiment of the invention, the nucleic acid molecules comprises of:

(1) an inducible promoter of a gene of a yeast of the genus Arxula, whose expression is associated with a change in cell morphology, and selected from the following nucleic acids:a nucleic acid with the sequence given in SEQ ID NO: 4;

(a) a nucleic acid with the sequence given in SEQ ID NO: 5;

(b) a nucleic acid with the sequence given in SEQ ID NO: 6;

(c) a nucleic acid with a sequence that is at least 50% identical with one of the sequences given in (a), (b) or (c);

(d) a nucleic acid, which hybridises with the complementary strand of one of nucleic acids given in (a), (b) or (c);

(e) a derivative one of the nucleic acids given in (a), (b) or (c) obtained by substitution, addition and/or deletion of one or several nucleotides;

(f) a fragment of one of nucleic acids given in (a) to (f), which retains the function of the inducible promoter;

(g) a combination of several nucleic acids given in (a) to (g), whereby the sequences of the nucleic acids can be the same or different; or

(2) a nucleic acid with a sequence that is complementary to one of the nucleic acids given in (a) to (h).

A change in cell morphology (yeast⇄mycelium) in accordance with the invention takes place in the cells of Arxula adeninovorans in the logarithmic growth phase at the latest 2 hours after one of the following changes in cultivation conditions:

change in cultivation temperature (<42° C.—yeast cells, 42° C.—pseudomycelium, >42° C.—mycelium),

addition of Cd²⁺ to cultivation medium (e.g. 0.1 mM Cadmium sulphate)

addition of NaCl to cultivation medium (≧10%),

addition of tunicamycin (≧8 μg/ml),

change in oxygen concentration in the culture (anaerobic conditions).

According to the invention, the term “% identity” relates to identity at the DNA level, which can be determined according to known methods, e.g. computer-aided sequence comparison (Altschul et al. 1990).

The term “identity,” as known to one skilled in the art relates to the degree of closeness between two or more DNA molecules, defined by the agreement between the sequences. The percent of “identity” results from the percent of identical areas in two or more sequences, with consideration of holes or other sequence peculiarities.

The identity of related DNA molecules can be determined with the help of known methods. Normally, computer programs are used with algorithms taking into consideration of special requirements. Preferred methods for determining identity at first create the greatest agreement between the investigated sequences. Computer programs for determining the identity between two sequences comprises, but are not limited, to GCG program package, including GAP (Devereux et al. 1984); Genetics Computer Group University of Wisconsin, Madison, (Wis.)); BLASTP, BLASTN and FASTA (Altschul et al. 1990). The BLAST X program can be obtained from the National Centre for Biotechnology Information (NCB1) and from other sources. (BLAST Manual, Altschul S. et al., NCB NLM NIH Bethesda Md. 20894; Altschul et al. 1990). The known Smith Waterman algorithm can also be used to determine identity.

Preferred parameters for sequence comparison include the following:

| Algorithm: | Needleman and Wunsch (1970) |
|---|---|
| Comparison matrix: | matches = +10, mismatch = 0 |
| Gap value (Gap Penalty): | 15 |
| Gap length penalty: | 1 |

The GAP program is also suitable for use with respect to the above-mentioned parameters. The above-mentioned parameters are default parameters for nucleic acid sequence comparison.

Other exemplary algorithms, gap opening penalties, gap extension penalties, comparison matrices including those mentioned in the Program Manual, Wisconsin Package, Version 9 September 1997, can be used. The selection depends on the comparison to be performed and also on whether the comparison is to be done between pairs of sequences, in which case GAP or best fit is preferred, or between a sequence and a comprehensive sequence database, in which case FASTA or BLAST is preferred.

The feature “sequence, which hybridises with the complementary strand of a sequence according to (a), (b) or (c)” points to a sequence, which under stringent conditions hybridises with the complementary strand of a sequence with the features given in (a), (b) or (c). For example, hybridisations can be performed at 68° C. in 2× SSC or according to the protocol of the Dioxygenin-Labelling-Kit from Boehringer (Mannheim).

The nucleic acid given in (d) can also be at least 60%, 70%, 80% or 90% identical with one of the sequences in (a), (b) or (c). Especially preferably it is 95% identical with one of this sequences.

In another embodiment of the invention, the nucleic acid molecule comprises of:

(1) an promoter, which is inducible by changing the salt concentration in culture medium, and which is selected from the following nucleic acids:

(a) a nucleic acid with the sequence given in SEQ ID NO: 7;

(b) a nucleic acid with the sequence given in SEQ ID NO: 8;

(c) a nucleic acid with the sequence given in SEQ ID NO: 9;

(d) a nucleic acid with a sequence that is at least 50% identical with one of the sequences given in (a), (b) or (c);

(e) a nucleic acid, which hybridises with the complementary strand of one of the nucleic acids given in (a), (b) or (c);

(f) a derivative of one of the nucleic acids given (a), (b), or (c) obtained by substitution, addition and/or deletion of one or several nucleotides;

(g) a fragment of one of the nucleic acids given in (a) to (f), which retains the function of the inducible promoter;

(h) a combination of several of the nucleic acids given in (a) to (b), whereby the sequences of the nucleic acids can be the same or different; or

(2) a nucleic acid with a sequence that is complementary to the sequence of one of the nucleic acids given in (a) to (h).

The nucleic acid given in ( c) can also be at least 60%, 70%, 80% or 90% identical with one of the sequences in (a) or (b). Especially preferably, it is 95 identical with one of these sequences.

In another embodiment, the nucleic acid molecule comprises:

(1) an inducible promoter by changing the salt and/or Fe (II) ion concentration in culture medium, selected from the following nucleic acids:

(a) a nucleic acid with the sequence given in SEQ ID NO: 10;

(b) a nucleic acid with the sequence given in SEQ ID NO: 11;

(c) a nucleic acid with a sequence that is at least 50% identical with one of the sequences given in (a) or (b);

(d) a nucleic acid, which hybridises with the complementary strand of one of the nucleic acid given in (a) or (b);

(e) a derivate of one of the nucleic acids given in (a) or (b) obtained by substitution, addition and/or deletion of one or several nucleotides;

(f) a fragment of one of the nucleic acids given in (a) to (e), which retains the function of the inducible promoter;

(g) a combination of several of the nucleic acids given in(a) to (f), whereby the sequences of the nucleic acids can be the same or different; or

(2) A nucleic acid with a sequence that is complementary to the sequence of one of the nucleic acids given in (a) to (g).

The nucleic acid given in ( c) can also be at least 60%, 70%, 80%, or 90% identical with one of the sequences given in(a) or (b). Especially preferably it is 95% identical with one of these sequences.

Another nucleic acid molecule according to the invention comprises of:

(1) a promoter inducible by a toxic agent, selected from the following nucleic acids:

(a) a nucleic acid with the sequence given in SEQ ID NO: 13;

(b) a nucleic acid with the sequence given in SEQ ID NO: 14;

(c) a nucleic acid with a sequence that is at least 50% identical with the sequence given in (a) or (b);

(d) a nucleic acid that hybridises with the complementary strand of one of the nucleic acids given in (a) or (b);

(e) a derivative of one of the nucleic acids given in (a) or (b) obtained by substitution, addition and/or deletion of one or several nucleotides;

(f) a fragment of one of the nucleic acids given in (a) to (e), that retains the function of the inducible promoter;

(g) a combination of several of the nucleic acids given in (a) to (g), whereby the sequences of the nucleic acids can be the same or different; or

(2) a nucleic acid with a sequence that is complementary to the sequence of the nucleic acid given in (a) to (g).

The nucleic acid given in (c) can also be at least 60%, 70%, 80%, or 90% identical with one of the sequences given in (a) or (b). Especially preferably it is 95% identical with one of these sequences.

Another nucleic acid molecule made available by the invention comprises of:

(1) a promoter, inducible by changing the salt and/or oxygen concentration in the culture medium, selected from the following nucleic acids:

(a) a nucleic acid with the sequence given in SEQ ID NO: 12;

(b) a nucleic acid with a sequence that is at least 50% identical with one of the sequences in (a);

(c) a nucleic acid that hybridises with the complementary strand of the nucleic acid given in (a);

(d) a derivative of one of the nucleic acid given in (a) obtained by substitution, addition, and/or deletion of one or several nucleotides;

(e) a fragment of one of the nucleic acids given in (a) to (d), which retains the function of the inducible promoter;

(f) a combination of several of the nucleic acids given in (a) to (e), whereby the sequences of the nucleic acids can be the same or different; or

(2) a nucleic acid with a sequence that is complementary to one of the nucleic acids given in (a) to (f).

The nucleic acid given in (b) can also be at least 60%, 70%, 80% or 90% identical with the sequence given in (a). Especially preferably it is 95% identical with this sequence.

Another nucleic acid molecule made available by the invention comprises of:

(a) a promoter inducible by xylit, xylose, sorbite and/or galactose, selected from the following nucleic acids:

(b) a nucleic acid with the sequence given in SEQ ID NO: 18;

(c) a nucleic acid with a sequence that is at least 50% identical with the sequence given in (a);

(d) a nucleic acid that hybridises with the complementary strand given in (a);

(e) a derivative of the nucleic acid given in (a) obtained by substitution, addition and/or deletion of one or several nucleotides;

(f) a fragment of one of the nucleic acids given in (a) to (d), which retains the function of the inducible promoter;

(g) a combination of several of the nucleic acids given in (a) to (e), whereby the sequences of the nucleic acids can be identical or different; or

(2) a nucleic acid with a sequence, that is complementary to the sequence of one of the nucleic acids given in (a) to (f).

The nucleic acid given in (b) can also be at least 60% , 70%, 80% or 90% identical with the sequence given in (a). Especially preferably it is 95% identical with this sequence.

Another nucleic acid molecule made available by the invention comprises of:

(1) a constitutive promoter, selected from the following nucleic acids:

(a) a nucleic acid with the sequence given in SEQ ID NO: 17;

(b) a nucleic acid with a sequence having at least 50% identity with the sequence given in (a);

(c) a nucleic acid that hybridises with the complementary strand of the nucleic acid given in (a);

(d) a derivative of the nucleic acid given in (a) obtained by substitution, addition and/or deletion of one or several nucleotides;

(e) a fragment of one of the nucleic acids given in (a) to (d) that retains the function of the constitutive promoter;

(f) a combination of several of the nucleic acids given in (a) to (e), whereby the sequences of the nucleic acids can be identical or different; or

(2) a nucleic acid with a sequence that is complementary to the sequence of one of the nucleic acids given in (a) to (f).

The nucleic acid given in (b) can also be at least 60%, 70%, 80% or 90% identical with the sequence given in (a). Especially preferably it is 95% identical with this sequence.

The nucleic acid molecule according to the invention can comprise of a nucleic acid sequence for a heterologous gene that is under the transcriptional control of the promoter. A “heterologous gene” as used here is a coding nucleic acid sequence, derived from another gene than the promoter used to control its transcription. The coding nucleic acid sequence can come from the yeast genus Arxula or from another organism. Examples for expressable heterologous genes are, among others, the genes for insulin, phytase, HGH, hirudin, lactoferrin or G-CSF.

The invention also makes available an expression vector, which comprises at least a nucleic acid molecule according to the invention. Preferably, the expression vector contains at least one marker gene. In a preferred embodiment, the expression vector according to the invention contains as marker gene the ALEU2 gene from Arxula adeninivorans or a variant. Especially preferably, the marker gene has the sequence given in SEQ ID NO: 15 or SEQ ID NO: 16. Other marker genes, such as the LYS2 gene from S. cerevisiaeor A. adeninivorans, or the AILV1-gene from A. adeninivorans can also be used.

The expression vector according to the invention can also comprise a sequence that makes possible the integration of the vector of part of the vector into the genome of a host cell of the yeast genus Arxula, for example a sequence from the 25S-rDNA of a yeast from the Arxula genus, especially A. adeninivorans . In another embodiment, the expression vector comprises a sequence, which makes possible the replication of the vector in a host cell of the Arxula yeast genus, such as ARS sequences from S. cerevisiae, Schwanniomyces occidentalis, Hansenula polymorpha, Arxula adeninivorans, Yarrowia lipolytica, Picha pastoris, Kluyveromyces lactis or Debaryomyces hansenii.

Furthermore, the invention makes available a host cell, containing a nucleic acid molecule according to the invention or an expression vector according to the invention. Preferably, the host cell belongs to the Arxula yeast genus. The yeast Arxula adeninivorans is especially preferred as host cell.

The invention also makes available a kit, which comprises of:

(a) an expression vector according to the invention that is suitable for cloning of a nucleic acid, which encodes a heterologous protein, and

(b) a host cell for induction of the promoter and synthesizing the heterologous protein.

The nucleic acid molecule, the expression vector, the host cell and the kit according to the invention can be used for expression of a gene under the control of the promoter or for synthesizing of one or more proteins.

### EXPERIMENTAL EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

Materials and Methods

Bacterial strains and culture conditions included E. coli LE392 (supE supF, hsd R r) (Sambrook et al. 1989) and E. coli TOP F′[mcraΔ (mmr-hsdRMS-mcrBC) F80ΔlacZDM15 DlacX74 deoR recA1 araD139 Δ(ara leu) 7697 galU galK 1⁻ rspL and A1 nupG F]—Invitrogen/USA. LB medium (Sambrook et al. 1989) was used for cultivation of strains. Yeast strains and culture conditions included A. adeninivorans LS3 (wild type strain) (Kunze and Kunze 1994), A. adeninivorans 135 (mutant with altered dimorphism) (Wartmann et al. 2000) and A. adeninivorans G1211 [ALEU2] (Samsonova et al. 1996). Yeast was cultivated in a complex (full medium—YEPD medium) or in a selective (yeast minimum medium—YMM) medium with 1% or 2% glucose or maltose as C source (Rose et al. 1990; Tanaka et al. 1967).

Transformation of A. adeninivorans

The transformation method for A. adeninivorans is comprehensively described in Rösel and Kunze (1998) or in Wartmann et al. (1998).

Northern Blot Analysis

RNA of A. adeninivorans was isolated according to the method described by Stoltenburg et al. (1995). In the case of agarose gel electropheresis, the method described according to Sambrook et al. (1989) was used. The RNA hybridisation was carried out at 42° C. according to the manual for GeneScreen nylon membranes delivered by NEN Research Products/USA.

Southern Blot Analysis

DNA of A. adeninivorans was prepared according to the method described by Kunze et al. (1987). The DNA fragments used as radioactive probe were labelled with [α−³²P]dATP (Amersham, UK) with use of a “Random Primer Labelling Systems” (Gibco, USA) according to the information from the manufacturer. Hybridisation was done at 65° C.

Western Blot Analysis

The procedure described by Wartmann et al (2000) was used for Western blot analysis of GFP, Gaap, and lacZp. HSA was detected with the anti-HSA antibody of Biotrend Chemikalien GmbH, BRD.

### Example 1

### The Constitutive ARFC3-Promotor

The yeast replication factor C (RF-C) is a multipolypeptide complex, composed of 5 subunits, coded by 5 different genes. One of these is the ARFC3 gene, which was isolated from the yeast A. adeninivorans . Besides the coding area, the ARFC3 promoter was also isolated and tested for its suitability for heterologous expression.

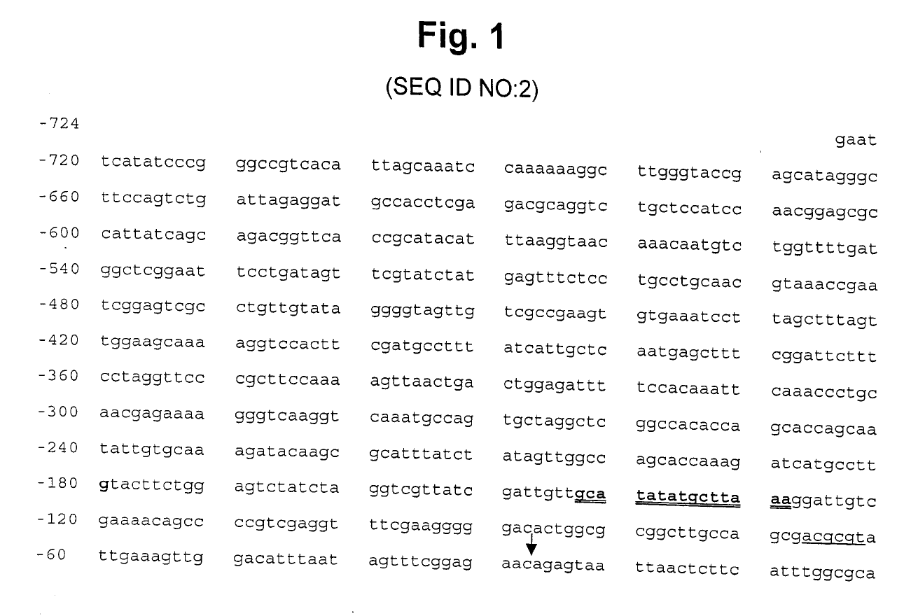

The promoter region of the ARFC3 gene comprises a TATA box (position −143 to −129) and in the region −67 to −82, a typical MluL location for the RFC gene. With the help of a 5′ RACE (Gibco, USA), the position −27 was determined as transcription start point (SEQ ID NO: 1, FIG. 1).

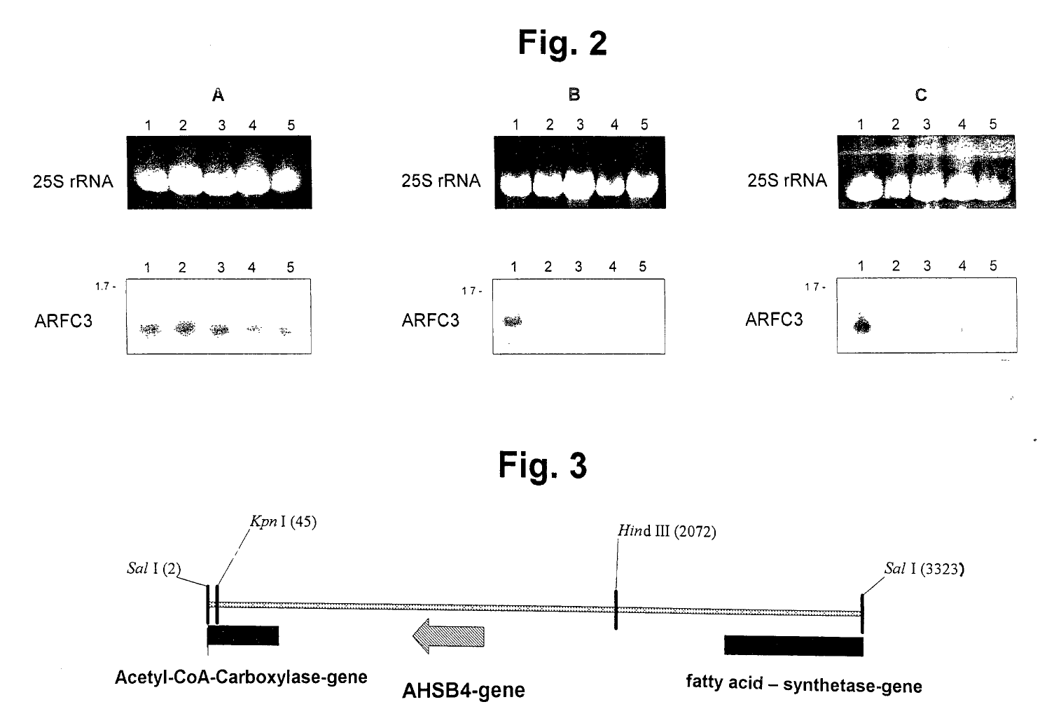

The ARFC3 transcript was already detectable after 20 hours of cultivation in all salt-free media (FIG. 2A). The determined transcript concentrations remain constant during the entire 70-hour cultivation. Thus, the ARFC3 promoter is a constitutive promoter. Adding salt (≧5% NaCl) leads to a reduction in the ARFC3 transcript concentration (FIG. 2 B, C).

### Example 2

### The Constitutive AHSB4 Promoter

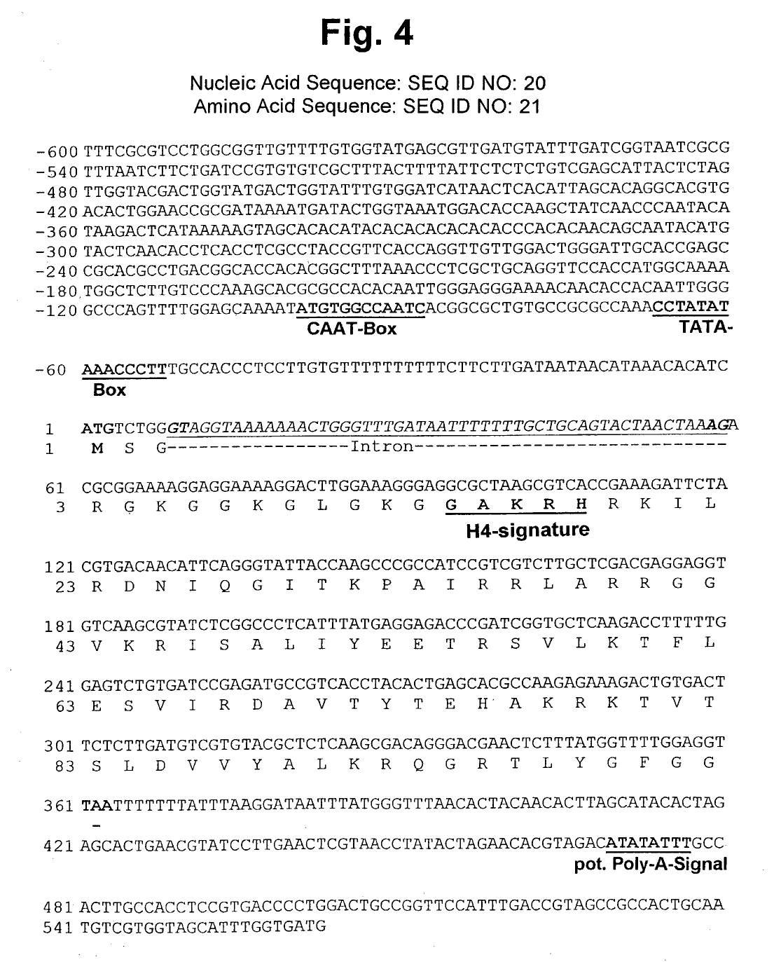

The AHSB4 gene from A. adeninivorans, which encodes the histon H4.2, was isolated as 3323 bp size Sal/Sal/fragment (FIG. 3). The gene comprises a 360-nucleotide size ORF, which encodes 120 amino acids. This ORF is interrupted by a 58 bp size intron. Within the amino acid sequence, the H4 signature was identified on the basis of homology comparison. In the 600 bp size promoter region of this gene, a CAAT box (position -89 -100) and the TATA box (position −67-−53) could be identified (FIG. 4).

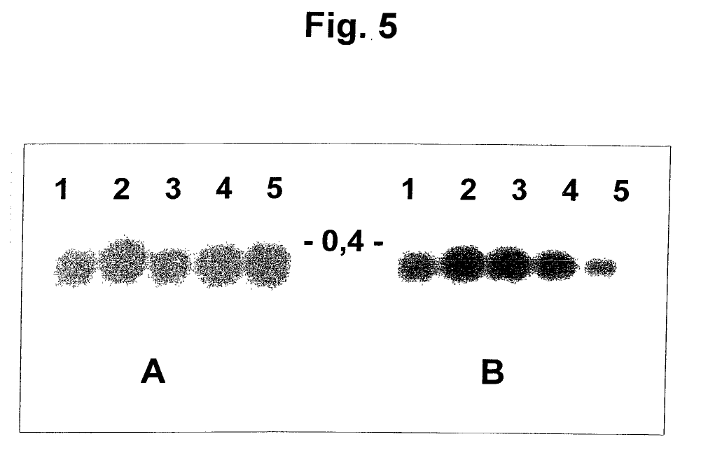

The activity of the AHSB4 promoter was analysed with the help of Northern hybridisation. For this purpose, A. adeninivorans LS3 yeast cells were cultivated in YMM (yeast minimum medium) with 2% maltose for 20, 36, 40, 60, and 70 hours, harvested and after isolation of the total RNA and Northern hybridisation, their AHSB4 transcript accumulation was determined. A constitutive expression of this gene was detected (FIG. 5A). Also adding 5% salt to YMM does not change the AHSB4 transcript accumulation or changes it only slightly (FIG. 5B).

### Example 3

### The Inducible GAA Promoter

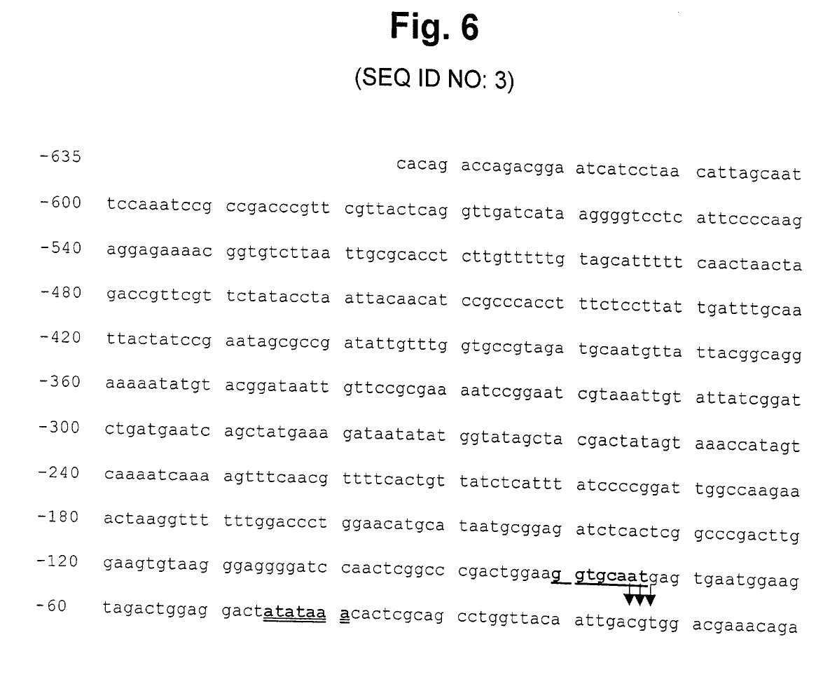

The yeast A. adeninivorans can, among others, use starch as C source. For this, it secretes a glucoamylase in cultivation medium, which cleaves the starch into glucose that is absorbed by the yeast and used as C source. This glucoamylase is coded by the GAA gene, isolated from the yeast A. adeninivorans LS3. The GAA promoter was tested for its suitability for heterologous expression. The promoter region of this gene comprises a TATA box (position −47 to −40) and a potential CAAT box (position −81 to −74). The transcription start point determined with the help of primer extension (Promega, FRG) is at the positions −15, −14, and −13 (SEQ ID NO: 3, FIG. 6).

The GAA promoter was characterised with the help of Northern hybridisation. For this purpose, A. adeninivorans yeast cells and mycelium of wild type strain LS3 or a mutant with altered dimorphism (A. adeninvorans 135; Wartmann et al. 2000) was cultivated at 30° C. and 45° C. in YMM with glucose, maltose and starch as C source. The cells were harvested after a corresponding period and the total RNA isolated. It was used for Northern hybridisation, whereby as radioactively labelled probe, a 1.6 kbp BamHI/EcoRI-DNA fragment with the GAA gene was used.

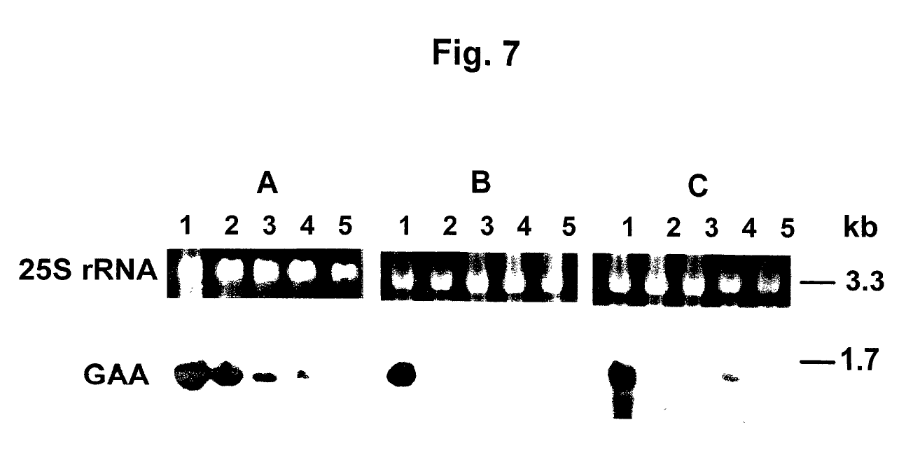

The maximum GAA transcript concentration was already reached in YMM with maltose or starch as C source after 20 hours of cultivation, both in the yeast cells and mycelium cultures. It dropped during further cultivation and after 45 hours and 60 hours of cultivation, no GAA transcript could be detected (FIGS. 7A, B, C). This course was neither influenced by the cell morphology nor by the cultivation temperature. In contrast to this, when glucose was used as C source during the entire cultivation, no GAA transcript was detectable. This is thus a GAA promoter, i.e. an adjustable promoter, which is activated as a function of the C source and cell morphology (e.g. maltose, starch, mycelium) or inhibited (e.g. glucose-yeast cells).

### Example 4

### The Inducible AACP10 Promoter

The AACP10 gene was isolated in the search for genes, expressed as a function of the morphology. This gene comprises an ORF of 1443 bp, which encodes a 52.1 kDa protein (481 amino acids). This protein has a 17.3% homology to lysosomal acid phosphatase (PPAL_RAT) of rats (Rattus norvegicus) or a 18.8% homology to lysosomal acid phosphatase ACPH-1 of Drosophila (CDPROT33, Intelligenetics, USA).

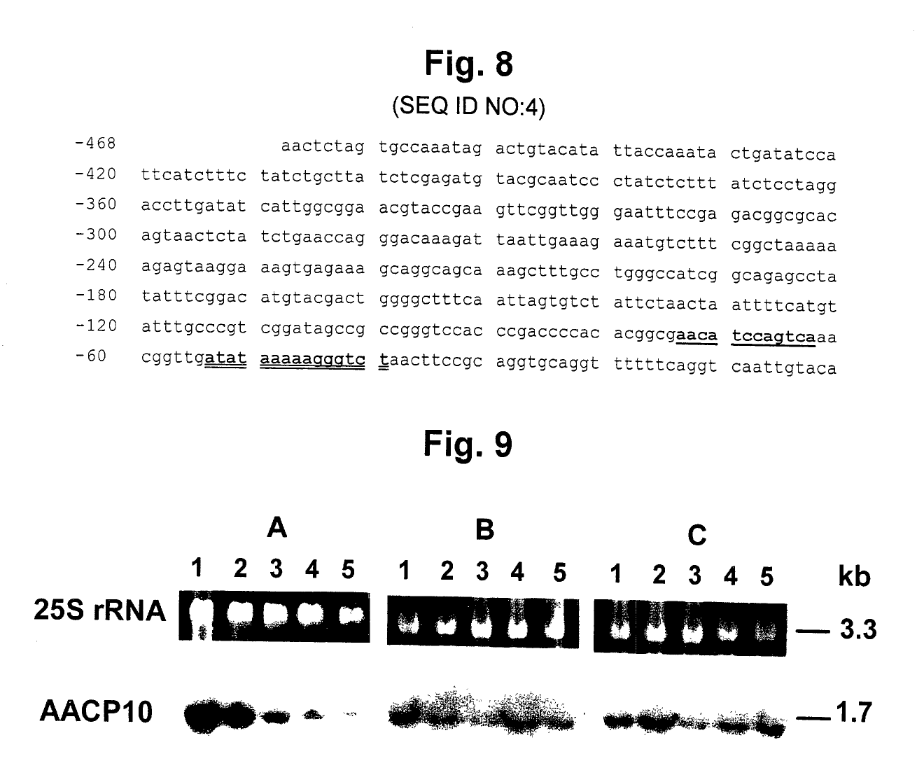

The promoter region including 478 nucleotides of the AACP10 gene comprise a TATA box (position −54 to −39) and a CAAT box (position −4 to −63) (SEQ ID NO: 4, FIG. 8).

The activity of the AACP 10 promoter was determined by means of Northern hybridisation. For this, A. adeninvorans yeast cells and mycelium of wild type strain LS3 or of the mutant with altered dimorphism (A. adeninivorans 135; Wartmann et al. 2000) was cultivated at 30° C. and 45° C. in YMM with glucose, maltose and starch as C source. The cells were harvested after a corresponding period and the total RNA isolated. This was used for Northern hybridisation, whereby a 1.5 kbp HindIII/SalI-DNA fragment with the ACP10gene was used as radioactively labelled probe.

The maximum AACP10transcript concentration was already detectable (FIG. 9A) in the yeast cell culture A. adenininvorans LS3-30° after 20 hours of cultivation in YMM with maltose as C source. During further cultivation it dropped. However, after 70 hours of cultivation, AACP10 transcripts could still be detected. In contrast, this gene gene is only slightly expressed (FIGS. 9B, C) in both mycelium cultures LS3-45° C. and 135-30° C. AACP 10 transcripts can only be identified after long detection of hybridized nitro-cellulose filter. Only the morphology influences the expression of this gene, but not temperature. For example, no difference can be found with the respect to transcript accumulation between the two mycelium cultures cultivated at different temperatures 45° C. (FIG. 9 B) and 135-30° C. (FIG. 9C). The same results were obtained after using YMM with other C sources (e.g. glucose) (FIG. 9). Thus, this is an adjustable AACP10 promoter, which can be activated or inhibited as a function of the morphology of the Arxula culture (yeast cell, mycelium).

### Example 5

### The Inducible APCR17 Promoter

In the search for genes, the APCR17 gene was isolated, which is expressed as a function of morphology. This gene comprises ORF of only 384 bp, which encodes a 14.7 kDa protein (128 amino acids). The protein Apcr17p has a potential transmembrane domain at the N-termination (type II membrane protein) and a potential mitochondrial signal sequence. Since no homologous sequences could be found to the APCR17 gene at the amino acid level yet, it is not possible to assign this gene to already known genes.

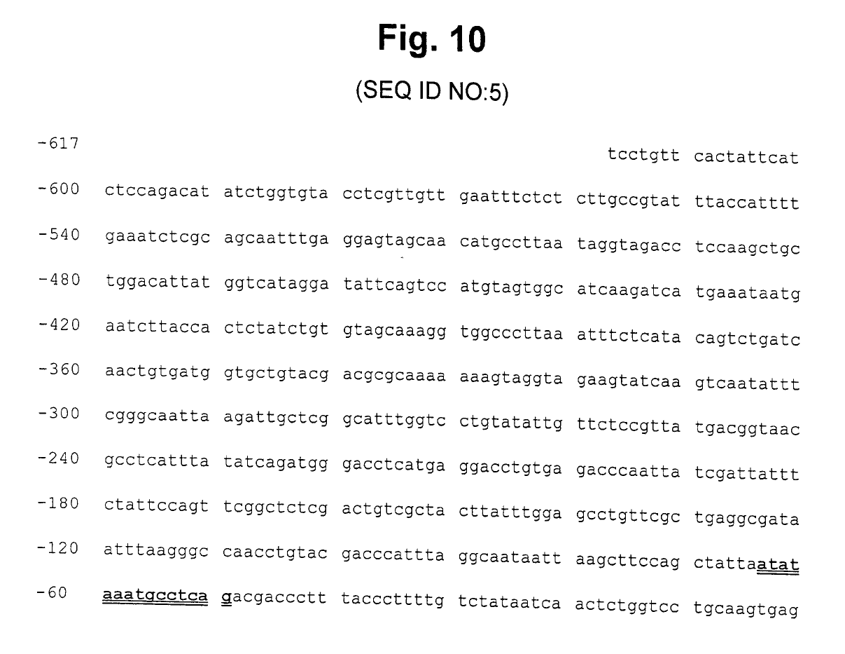

The promoter of the APCR17 gene was isolated. In this area including 617 nucleotides, the TATA box was determined between the positions −64 and −49 (SEQ ID NO: 5, FIG. 10).

The activity of the APCR17 promoter was analysed by means of Northern hybridisation. For this purpose, A. adeninivorans yeast cells and mycelium of wild type strain 1S3 or of a mutant with altered dimorphism (A. adeninivorans 135; Wartmann et al. 2000) was cultivated at 30° C. and 45° C. in yeast minimum medium (YMM) with maltose as C source. The cells were harvested after a corresponding period and the RNA was isolated. This was used for Northern hybridisation, whereby a 0.3 kbp EcoRI/HindIII-DNA fragment with the APCR17 code was used as radioactively labelled probe.

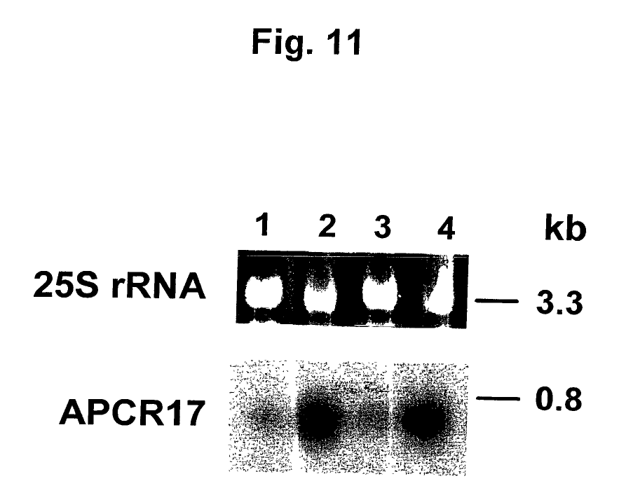

An APCR17 transcript could be detected in the mycelium culture A. adeninivorans LS3-45° C. after 40 hours of cultivation in YMM with maltose as C source. In contrast, this gene is not expressed in yeast cell cultures. Even after long detection of the hybridised nitrocellulose filter with the radioactively labelled APCR17 probe, it was not possible to identify APCR17 transcripts (FIG. 11). Thus, the APCR17 promoter must be called an adjustable promoter. In contrast to the AACP10 promoter, it is however only active in the mycelium culture, while APCP17 transcripts were not detectable in the mycelium culture.

### Example 6

### The Inducible APRE4 Promoter

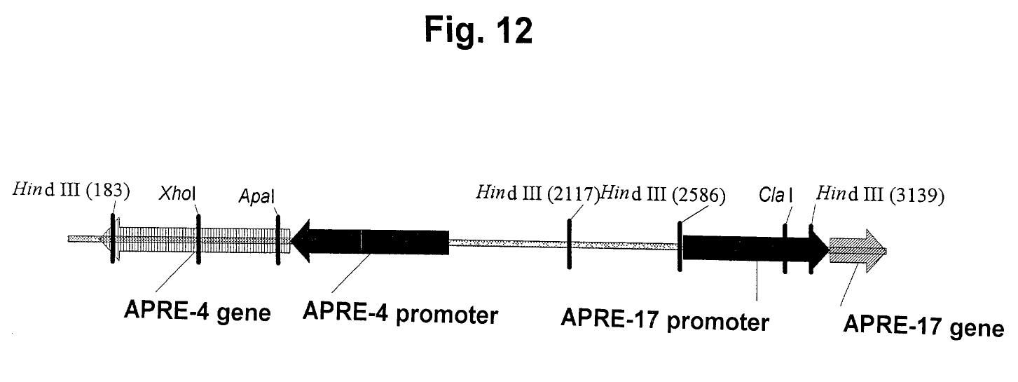

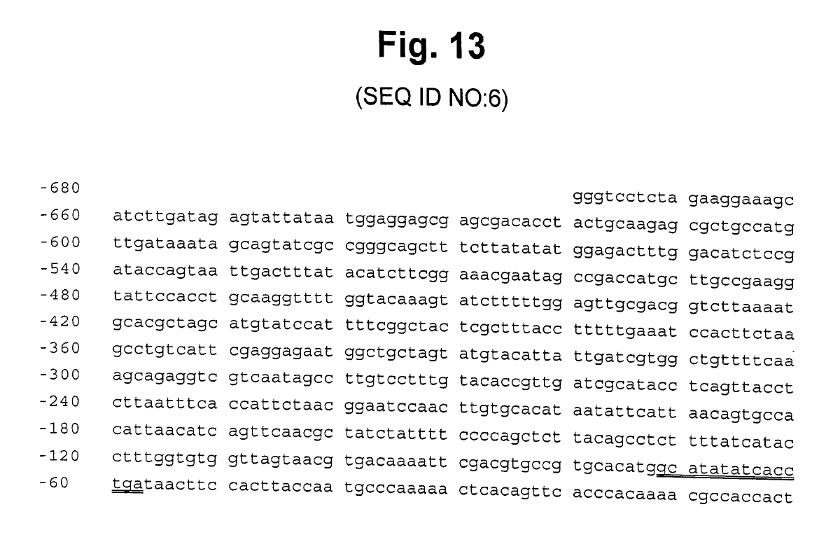

The APRE4 gene, which is directly besides the APCR17 gene on the chromosomal DNA of A. adeninivorans LS3 (FIG. 12), was also isolated as a gene, which is expressed as a function of the morphology. This gene comprises an ORF of 801 bp, which encodes a 29.9 kDa protein (267 amino acids). The protein APRE4p has a 51.5% homology to the proteasom-β-subunit Pre4p of S. cerevisiae is located in cytoplasma and in the cell nucleus. The promoter region of the APRE4 gene including 674 nucleotides comprises of a TATA box (position −115 to −100). A CAAT box cannot be identified (SEQ ID NO: 6; FIG. 13).

Again, the activity of the APRE4 promoter was determined with Northern hybridisation. For this purpose, A. adeninivorans yeast cells and mycelium of the wild type strain LS3 or the mutant with altered dimorphism (A. adeninivorans 135; Wartmann et al. 2000) was cultivated at 30° C. and 45° C. in YMM with glucose, maltose and starch as C source. The cells were harvested after a corresponding period and the total RNA was isolated. This was used for Northern hybridisation, whereby a 0.8 kbp ApaI/HindIII-DNA fragment with the APREA gene was used as radioactively labelled probe.

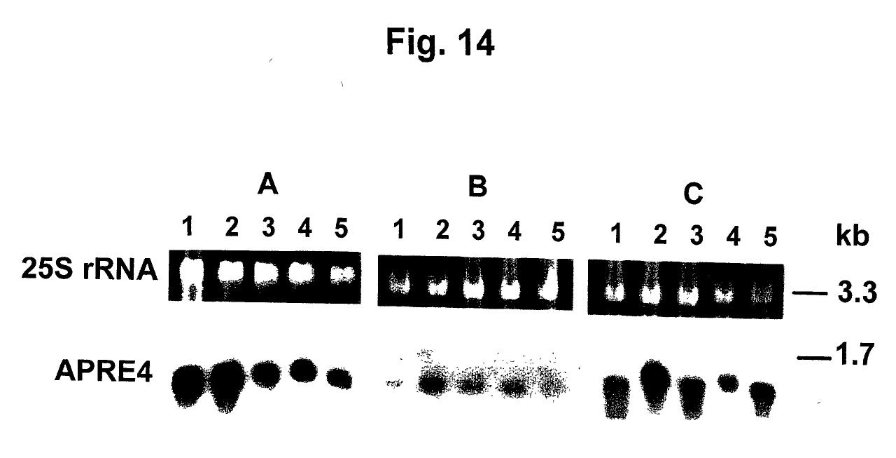

The maximum APRE4 transcript concentration was already reached after 20 hours of cultivation in YMM with maltose as C source (FIG. 14A). It remained at first constant during further cultivation, until it sank to a lower level after a cultivation period ≧45 hours. However, even after 70 hours of cultivation, APRE4 transcripts were still detectable. In contrast to this, this gene is expressed lesser in both mycelium cultures LS3-45° C. and 135-30° C. (FIGS. 14B, C). Northern hybridization signals can only be detected (FIG. 14B) after very long detection periods, especially in the culture LS3-45° C. However, somewhat more APRE4 transcripts can be determined in the mycelium culture 135-30° C. (FIG. 14C). Here weaker signals occur than with the yeast cell culture LS3-30° C., however, in comparison with the mycelium culture LS3-45° C. they are stronger. Thus, besides the morphology, the activity of the APRE4 promoter is especially influenced by the cultivation temperature. For example, maximum APRE4 promoter activities occur in yeast cells, cultivated at 30° C. On the other hand, a change in the C source of the YMM has no effects on the activity of this promoter.

### Example 7

### The AACE1 Promoter

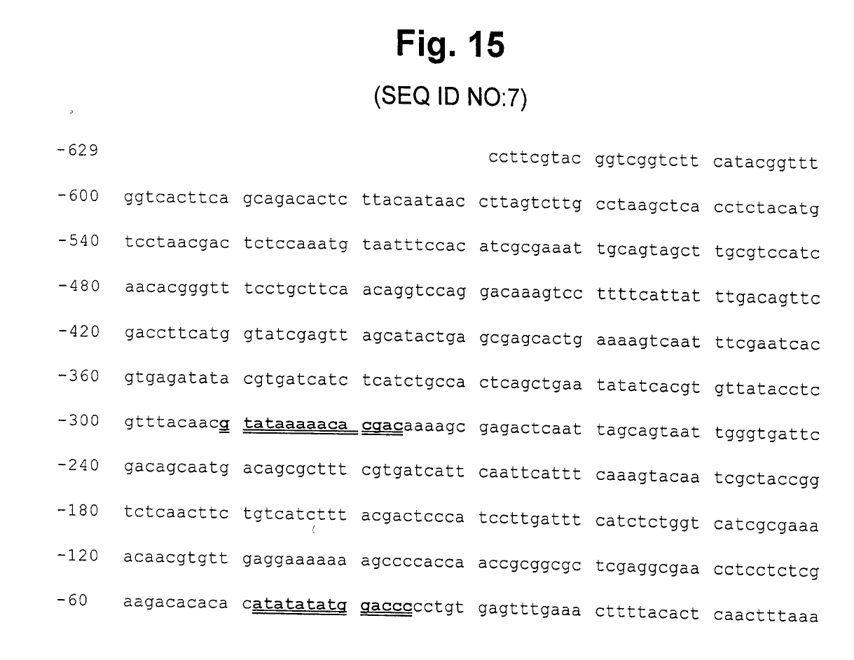

The AACE1 gene encodes an acetyl CoA-acetyltransferase of A. adeninivorans, which participates in the decomposition of fatty acids in mitochondrion. This enzyme cleaves the activated β keto acid with Coa to acetyl CoA (Kurihara et al. 1992). The promoter region of the AACE1 gene including 629 nucleotides contains two potential TATA boxes (position −291 to −277, −49 to −36) (SEQ ID NO: 1, FIG. 15).

The activity of the AACE1 promoter was analysed with the help of Northern hybridisation. For this purpose, A. adeninivorans yeast cells of the wild type strain LS3 or the mutant with altered dimorphism (A. adeninivorans 135; Wartmann et al. 2000) was cultivated at 30° C. and 45° C. in YMM with glucose, maltose and starch as C source at first for 40 hours in NaCl-free medium, centrifuged sterile and transferred to NaCl containing YMM (5% NaCl=end concentration). These cells were harvested after different times and the total RNA was isolated, which was used for Northern hybridisation. A 1.0 kbp HindIII/XhoI-DNA fragment with the AACE1 gene was used as radioactively labelled probe.

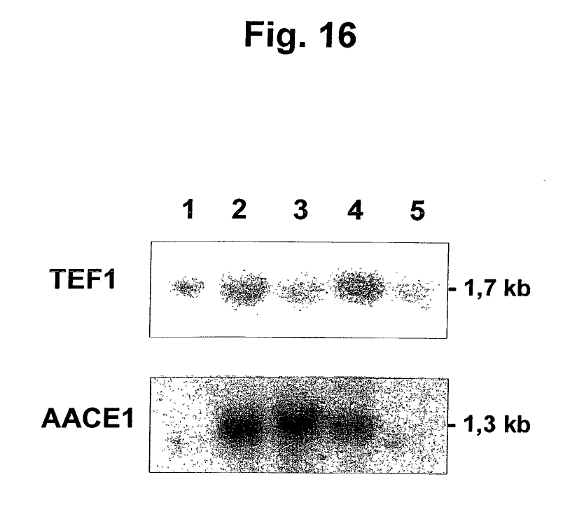

In contrast to the salt-free cultures, already after 30 minutes of cultivation of the Arxula cells in NaCl containing YMM, there is formation of AACE1 transcripts. The maximum transcript accumulation is reached after 1 hour of cultivation. Then, there is a decrease in AACE1 transcript concentration. After 4 hours of cultivation in salt-containing YMM, transcripts can only be detected in very low concentrations (FIG. 16). Thus, the AACE1 promoter is activated by adding NaCl. However, this activation is restricted with respect to time. The promoter activity reaches the initial state (salt-free medium) already after 4 hours. Morphology and temperature do not influence promoter activity or they influence it only slightly.

### Example 8

### The ATAL1 Promoter

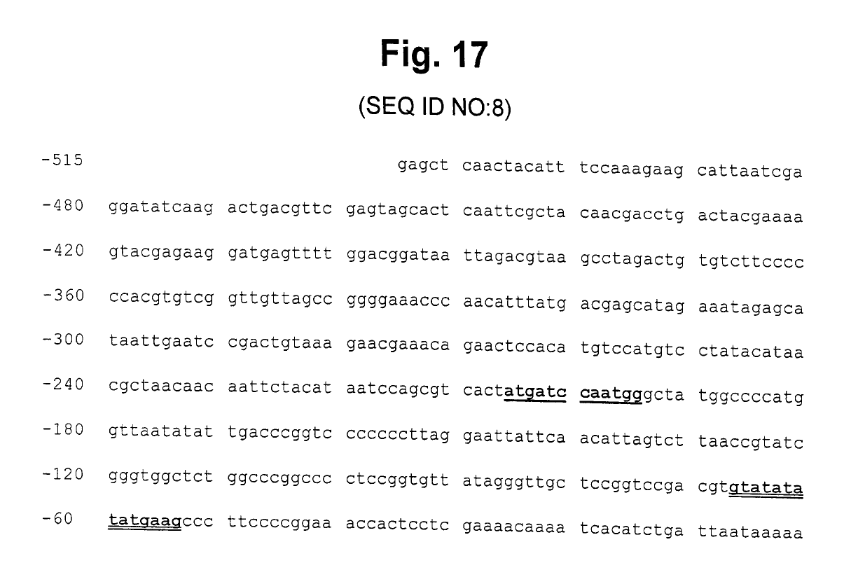

The ATAL1 gene encodes a transaldolase from A. adeninivorans, needed in the pentosephosphate pathway and having a high degree of homology to the transaldolase of S. cerevisiae (Johnston et al. 1997). The promoter region including 515 nucleotides of the ATAL1 gene contains a TATA box (position −67 to −54) and a potential CAAT box (position −206 to 195) (SEQ ID NO: 8; FIG. 17).

The ATAL1 promoter activity was analysed with the help of Northern hybridisation. For this purpose analogously to the AACE1 promoter investigation, A. adeninivorans yeast cells and mycelium from wild type strain LS3 or the mutant with altered dimorphism (A. adeninivorans 135; Wartmann et al. 2000) was cultivated at 30° C. and 45° C. in YMM with glucose, maltose and starch as C source at least in salt-free medium, then transferred to NaCl-containing YMM (5% NaCl=end concentration). Then the cells were harvested and the total RNA isolated. It was used for Northern hybridisation, whereby a 0.3 kbp EcoRI-DNA fragment with the ATAL1 gene was used as radioactively labelled probe.

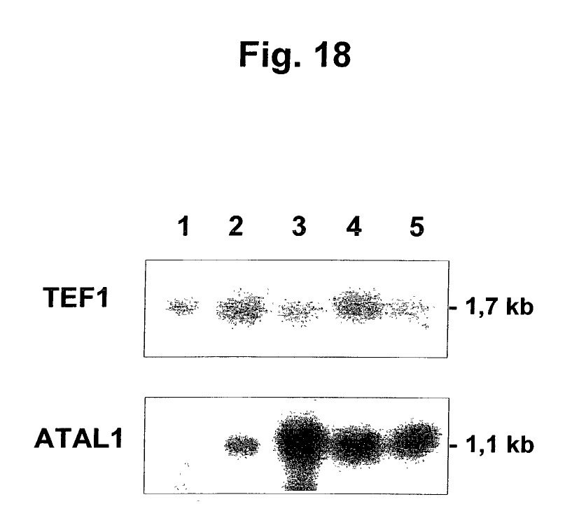

Already after 30 minutes of cultivation of the Arxula cells in NaCl-containing YMM, there is activation of the ATAL1 promoter as regards the ATAL1 gene. The maximum transcript accumulation is reached after 1 hour of cultivation and remains constant during the further 4-hour cultivation in salt-containing YMM (FIG. 18). Thus, the ATAL1 promoter, which is only slightly active in salt-free medium is activated by adding NaCl. In contrast, the morphology and temperature do not influence promoter activity or they influence it only very slightly.

### Example 9

### The AGLC7 Promoter

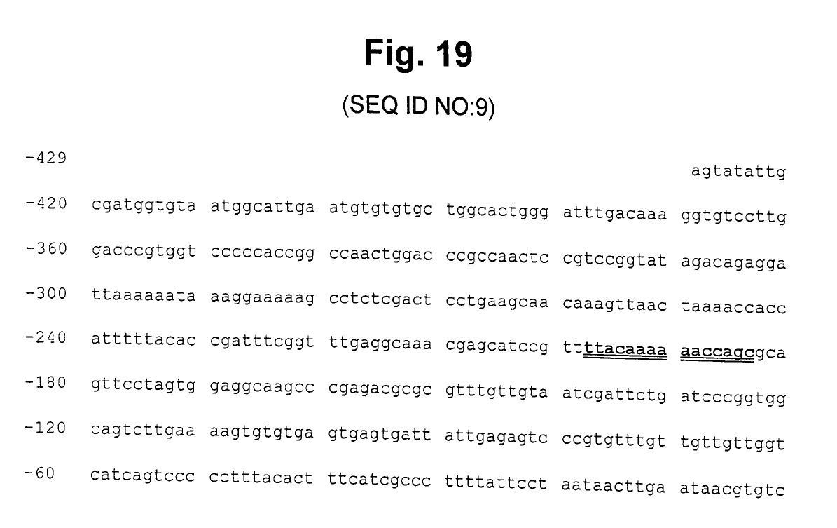

The AGLC7 gene encodes a protein phosphatase, that is located cytoplasmatically and has a high degree of homology to the protein phosphatase Glc7p from S. cerevisiae (Ramaswamy et al. 1998). In the promoter region of the AGLC7 gene including 429 nucleotides the TATA box could be identified between the position −198 to −184 (SEQ ID NO: 9; FIG. 19).

The activity of the AGLC7 promoter was analysed by means of Northern hybridisation. For this purpose, A. adeninivorans yeast cells and mycelium from wild type strain LS3 or the mutant with altered dimorphism (A. adeninivorans 135; Wartmann et al. 2000) was cultivated at 30° C. and 45° C. in YMM with glucose, maltose and starch as C source at least in salt-free medium, then transferred to NaCl-containing YMM (5% NaCl =end concentration). Then the cells were harvested and the total RNA isolated. It was used for Northern hybridisation, whereby a 0.4 kbp EcoRI/XbaI-DNA fragment with the AGLC7 gene was used as radioactively labelled probe.

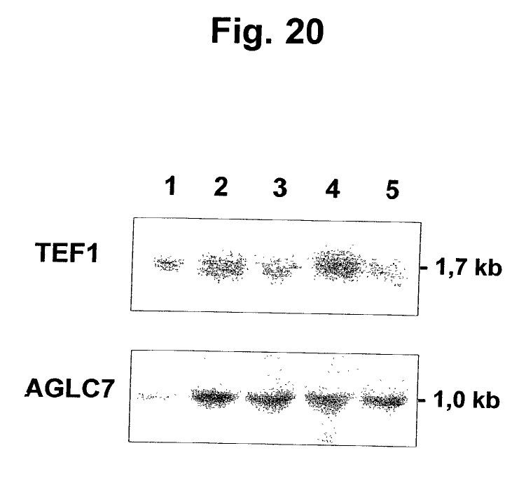

Thus, the AGLC7 promoter is active in salt-containing and salt-free YMM. However, if yeast cell cultures of mycelium cultures are transferred from a salt-free YMM to a salt-containing YMM, the AGLC7 promoter activity increases. These increased transcript concentrations remain constant during a 4-hour cultivation in salt-containing YMM (FIG. 20). Thus, the AGLC7 promoter can also be considered a promoter, of which the activity can be increased by adding salt to the YMM.

### Example 10

### The AFET3 Promoter

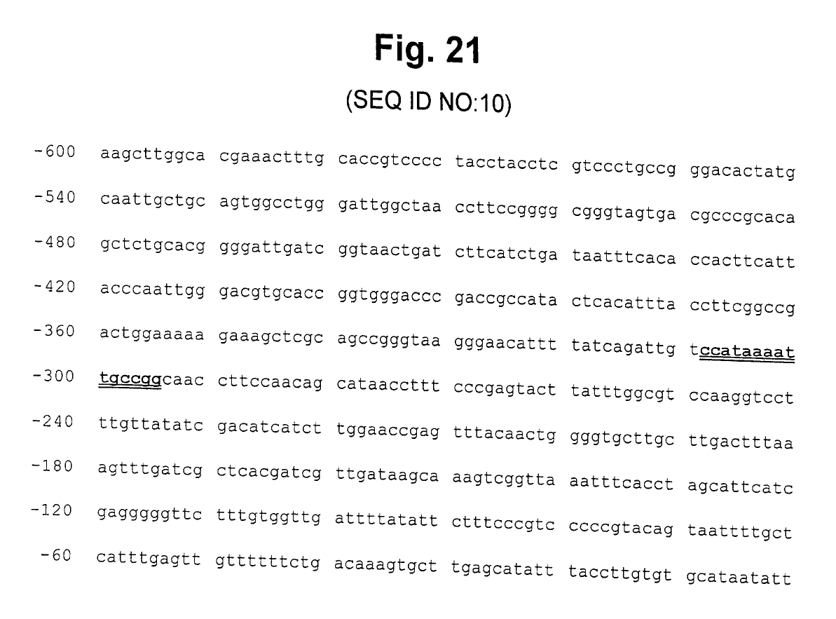

The yeast A. adeninivorans uses at least a “low affinity” and a “high affinity” system for the transport of iron ions. The Afet3 protein (Afet3p) that is coded by the AFET3 gene is a component of the “high affinity” system. This gene comprises of an ORF of 1845Bp, which encodes the 69.6 kDa Afet3p (615 amino acids). The protein has a 31% homology to the Fet3 protein of S. cerevisiae . The promoter region of the AFET3 gene comprising 600 nucleotides contains solely a TATA box (position −309 to −295) (SEQ ID NO: 10; FIG. 21).

The activity of the AFET3 promoter was determined via Northern hybridisation. For this purpose, A. adeninivorans yeast cells and mycelium from wild type strain LS3 or the mutant with altered dimorphism (A. adeninivorans 135; Wartmann et al. 2000) was cultivated at 30° C. and 45° C. in YMM with glucose, maltose and starch as C source with increasing Fe(II) concentrations. The cells were harvested after 40 hours and the total RNA was isolated. It was used for Northern hybridisation, whereby a 1.2 kbp Hindlll/SalI-DNA with the AFET3 gene was used as radioactively labelled probe.

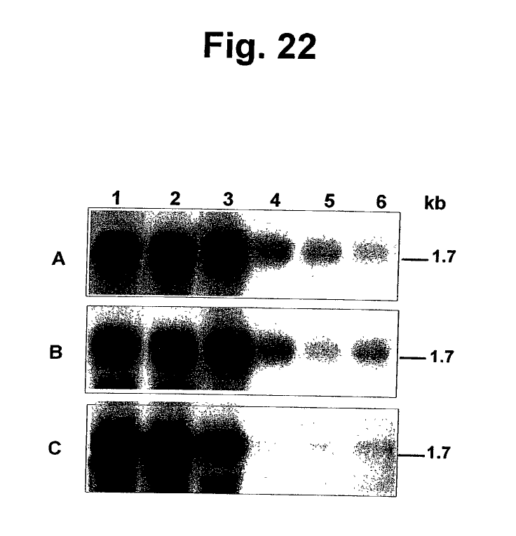

The highest AFET3 transcript concentrations were always reached when the media without FeEDTA were used. When FeEDTA is added to the cultivation medium, the transcript concentration decreases. At FeEDTA concentrations ≧5 μM FeEDTA, hardly any AFET3 transcript can be detected (FIG. 22). Thus, the AFET3 promoter can be regulated via the Fe(II) concentration. In contrast to this, change in the morphology (yeast cells-mycelium) or change in the C source (glucose, maltose, etc.) does not influence the AFET3 promoter activity.

### Example 11

### The AFTR1 Promoter

The Aftr1 protein that interacts with the Fet3 protein is another component of the “high affinity” systems and is located in the cytoplasm membrane. The corresponding AFTR1 gene comprises an ORF of 1230 bp, which encodes the 46.0 kDa Aftr1p (410 amino acids). This protein has a 45.3% homology to the Ftr1 protein of S. cerevisiae.

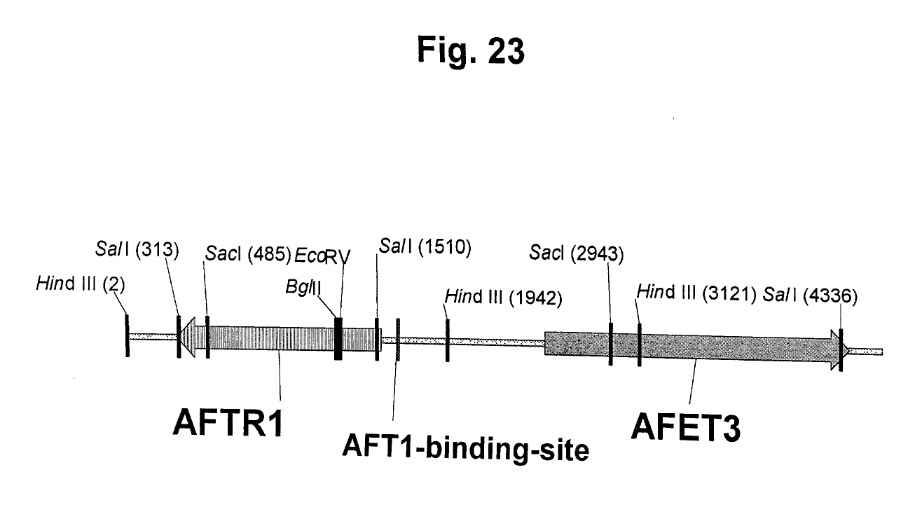

The location site of this gene is interesting. For example, the AFTR1 gene is directly next to the AFET3 gene. The promoters of both genes overlap. Since both genes are regulated via the Fe(II) concentrations, the AFT1 binding location between both genes influences both the expression of AFET3 and AFTR1 gene. Thus, this 1009 bp size DNA fragment can be used for simultaneous expression of two genes, whereby the strength of expression of both genes depends on the Fe(II) concentration (FIG. 23).

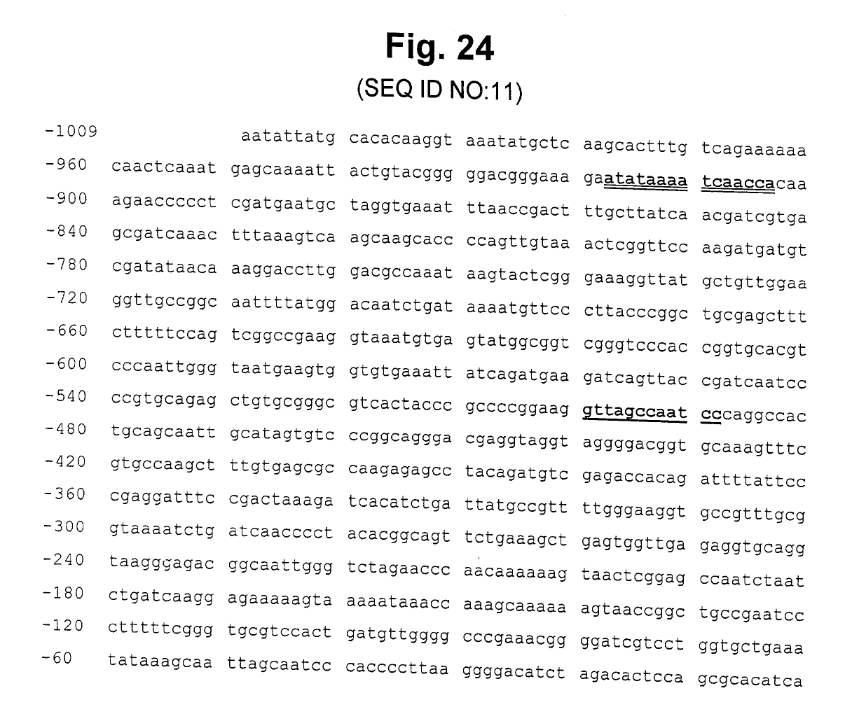

The promoter region of the AFTR1 gene including 1009 nucleotides contains a potential TATA box (position −918 to −904) and a CAAT box (position −500 tos −488) (SEQ ID NO: 11; FIG. 24).

The activity of the AFTR1 promoter was determined via Northern hybridisation. For this purpose, A. adeninivorans yeast cells and myceliums from wild type strain LS3 or the mutant with altered dimorphism (A. adeninivorans 135; Wartmann et al. 2000) was cultivated at 30° C. and 45° C. in YMM with glucose, maltose and starch as C source with increasing Fe(II) concentrations. The cells were harvested after 40 hours and the total RNA was isolated. It was used for Northern hybridisation, whereby a DNA fragment with the AFTR1 gene was used as radioactively labelled probe.

The highest AFTR1 transcript concentrations were reached in all three cultures when media without FeEDTA were used. If FeEDTA is added to the cultivation medium, the transcript concentration decreases. At FeEDTA concentration ≧5 μM FeEDTA, analogously to AFET3 gene, hardly any AFTR1 transcript can be detected. Thus, the AFTR1 promoter is also regulated via the concentration of Fe ions in the cultivation medium. Changes in the morphology (yeast cells, mycelium) or the C source used for cultivation (glucose, maltose, etc.) has no influence on the AFTR1 promoter activity.

### Example 12

### The AEFG1 Promoter

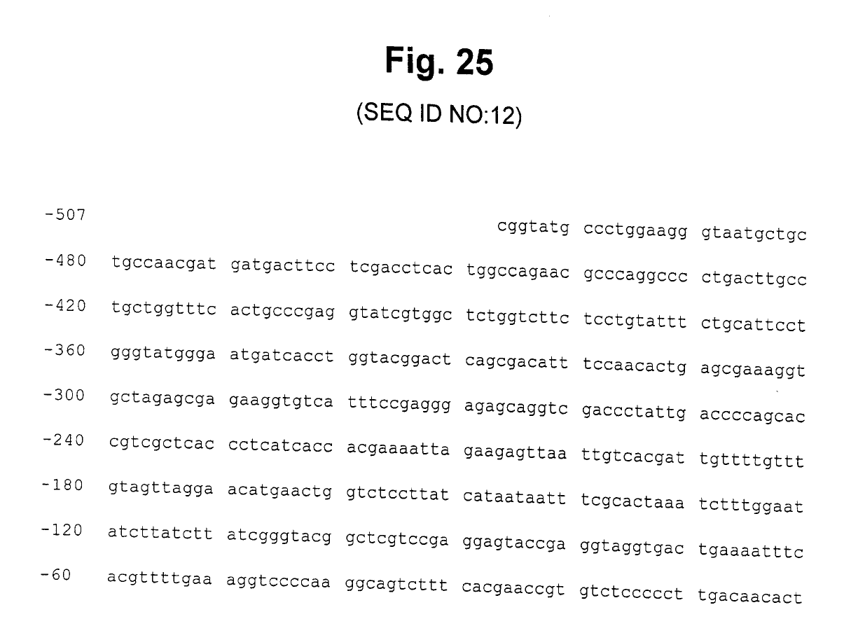

The 2271 bp size AEFG1 gene encodes the mitochondrial elongation factor G-1. This 83.9 kDa size protein (757 amino acids) contains a potential transmembrane domain on the C-termination (type Ib-membrane protein), having a potential mitochondrial signal sequence at the N-terminus and a 65.3% homology to Efg1p of S. cerevisiae. The promoter region of AEFG1 gene comprising of 507 nucleotides is represented in FIG. 25 (SEQ ID NO: 12).

The activity of the AEFG1 promoter was determined via Northern hybridisation. For this purpose, A. adeninivorans yeast cells and mycelium from wild type strain LS3 or the mutant with altered dimorphism (A. adeninivorans 135; Wartmann et al. 2000) were aerobically and anaerobically cultivated at 30° C. and 45° C. in YMM with glucose, maltose and starch as C source. The cells were harvested after 40 hours and the total RNA was isolated. It was used for Northern hybridisation, whereby a 2.1 kbp Hind/lll DNA fragment with the AEFG1 gene was used as radioactively labelled probe.

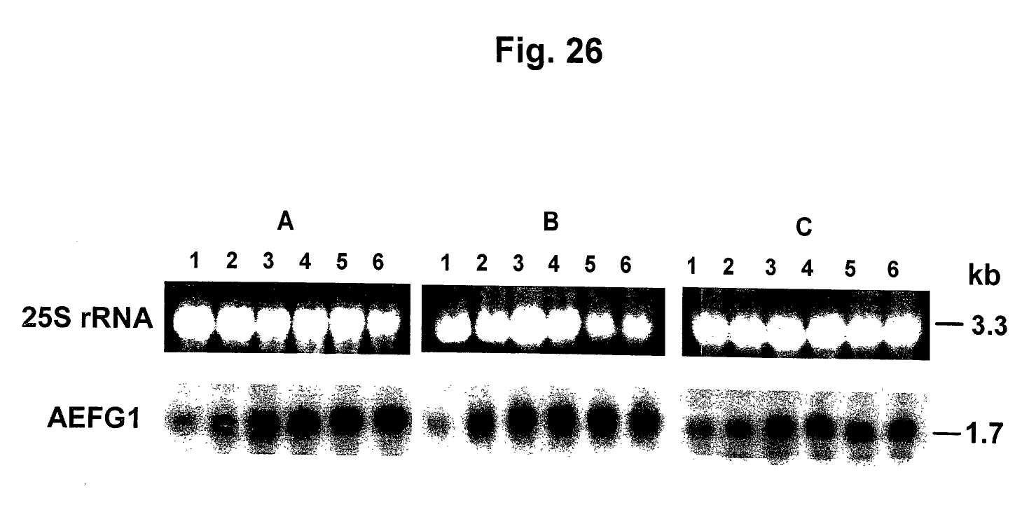

There is an increase in AEFG1 transcript concentrations after the shift from aerobic to anaerobic conditions. For example, the highest transcript concentrations were measured (FIG. 26) 24 hours after the shift, independently of the strain used. Thus, the AEFG1 promoter can be regulated via the shift from aerobic to anaerobic conditions. In contrast with this, a change in morphology (yeast cell—mycelium) or change in C source (glucose, maltose, etc.) has no influence on the AEFG1 promoter activity.

### Example 13

### ASTE6 Promoter

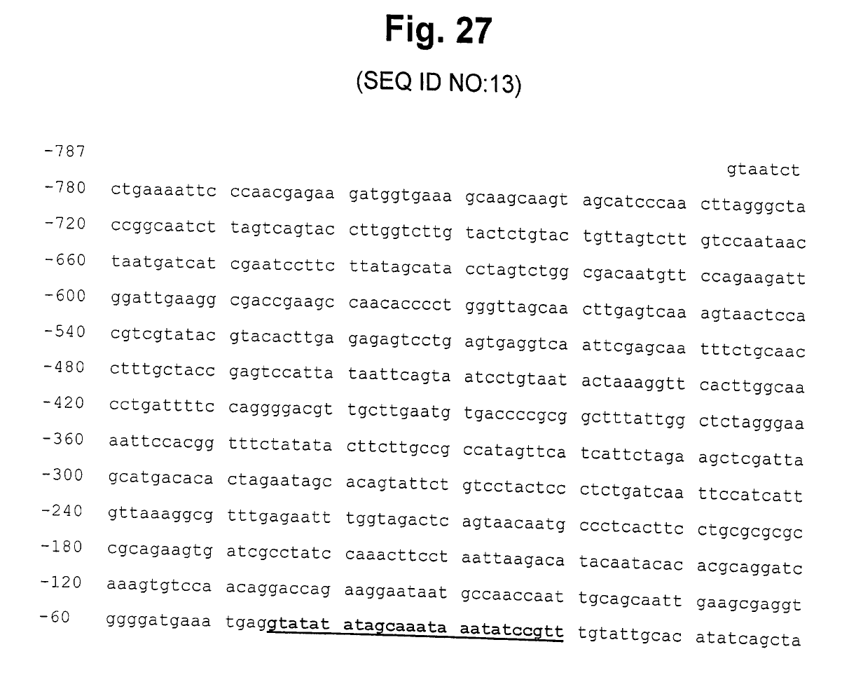

The yeast S. cerevisiae has a Ste6 protein, which transports the a-factor from the cells. In addition to the ATP/GTP binding regions, this protein belonging to the ABC transporters contains different transmembrane regions, which are very conserved in their sequence (Kuchler et al. 1989). Although no sexuality has been detected for A. adeninivorans, such a gene (ASTE6 gene) has been identified and isolated in Arxula. The Aste6p has a 31% homology to the amino acid sequence of Ste6p and contains all typical regions for this transporter (transmembrane domains, ATP/GTP binding regions). The promoter region of this gene comprises of three potential TATA boxes, located between the base positions −46 and −21 (SEQ ID NO: 13; FIG. 27).

The ASTE6 promoter was characterised with the help of Northern hybridisation. An ASTE6 transcript could only be detected when a toxic agent such as leptomycin B, cycloheximide, or chloramphenicol (≧0.008 μg/ml) was added to the medium. This effect was strain-independent. Thus, the ASTE6 promoter is an inducible promoter via toxic agent.

### Example 14

### The APMD1 Promoter

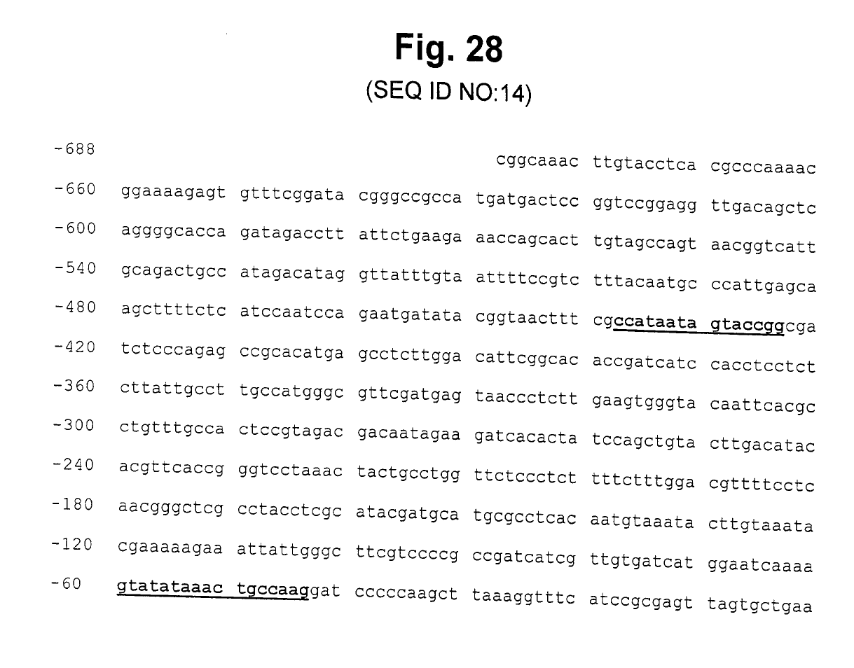

The PMD1 gene from Schizosaccharomyces pombe also encodes an ABC transporter that is responsible for the leptomycin B resistance of this yeast. This gene contains all typical conserved regions for such ABC transporters, such as ATP/GTP binding regions and different transmembrane regions (Nishi et al. 1992). Such a gene (APMD1 gene) was isolated from A. adeninivorans. It has a 45.9% homology to the Sch.pombe PMD1 gene at the amino acid level. The promoter region of the APMD1 gene comprises of three potential TATA boxes, located between the base positions −438 and −423, −60 and 45 or −58 and −43 (SEQ ID NO: 14; FIG. 28).

Northern hybridisations show that the APMD1 promoter is only active in the presence of leptomycin B, i.e. under these conditions a transcript can be detected. This effect is also strain independent. Thus, the APMD 1 promoter must also be considered an inducible promoter via leptomycin B.

### Example 15

### The AXDH Promoter

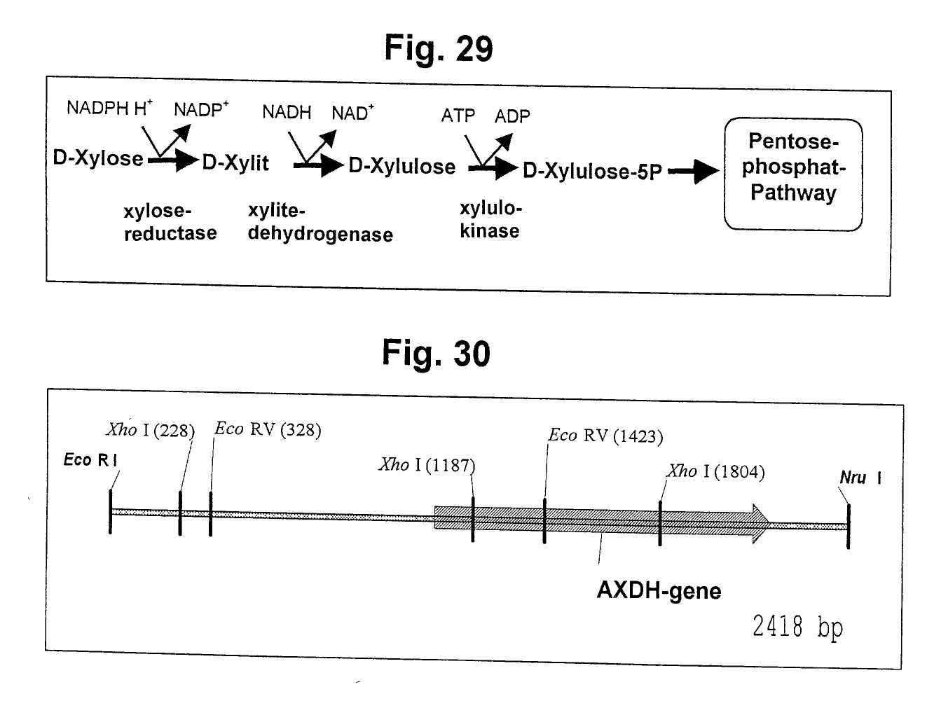

The yeast A. adeninivorans LS3 can use xylose and xylit as C source. For this, the enzymes xylose-reductase, xylit-dehydrogenase and xylulokinase are synthesized, which further hydrolises D-xylose intol D-xylit and D-xylulose-5-phosphate (FIG. 29).

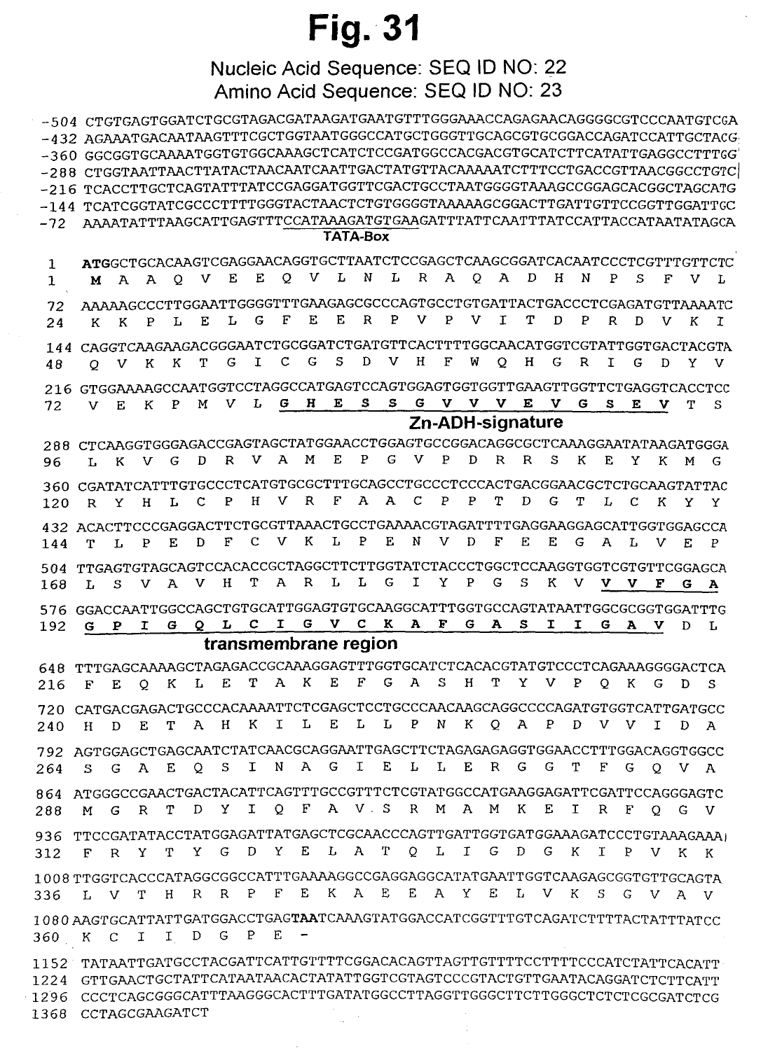

The enzyme xylit-dehydrogenase is encoded by the AXDH gene in the yeast A. adeninivorans LS3. A 2418 bp EcoRI-Nrul fragment (FIG. 30), containing the AXDH gene was isolated. The AXDH gene comprises of 1107 nucleotides, which code a membrane protein of 368 amino acids. Within the protein, the Zn-ADH signature and the transmembrane region could be identified on the basis of homology comparison. The 504 bp size promoter region of this gene comprises of a TATA box (position −50 to −36) (FIG. 31); SEQ ID NO: 22).

The AXDH promoter was characterised with the help of Northern hybridisations. For this purpose, A. adeninivorans LS3 yeast cells were cultivated at 30 ° in YMM with 2% glucose, galactose, sorbite, fructose, mannite, xylit and xylose. The cells were harvested after 24 h and 48 h and the total RNA isolated. It was used for Northern hybridisations, whereby a 0.7 kbp XhoI-DNA fragment with the AXDH gene was as radioactively labelled probe.

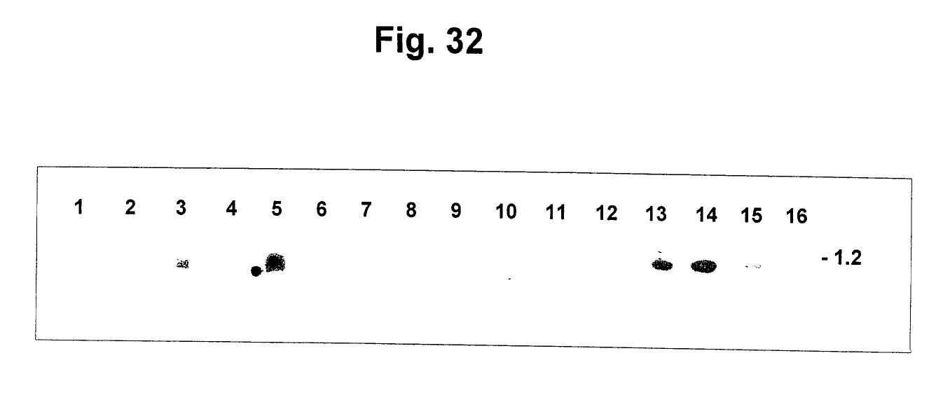

In contrast to the cultivations with glucose, fructose, maltose and mannite, when galactose, sorbite and xylose were used, hybridisation signals were detected. While the AXDH transcript is accumulated in cells only after 24 hours of cultivation in galactose, sorbite and xylose medium, AXDH transcripts could still be detected after 48 hours of cultivations in the case of cells growing in xylit (FIG. 32).

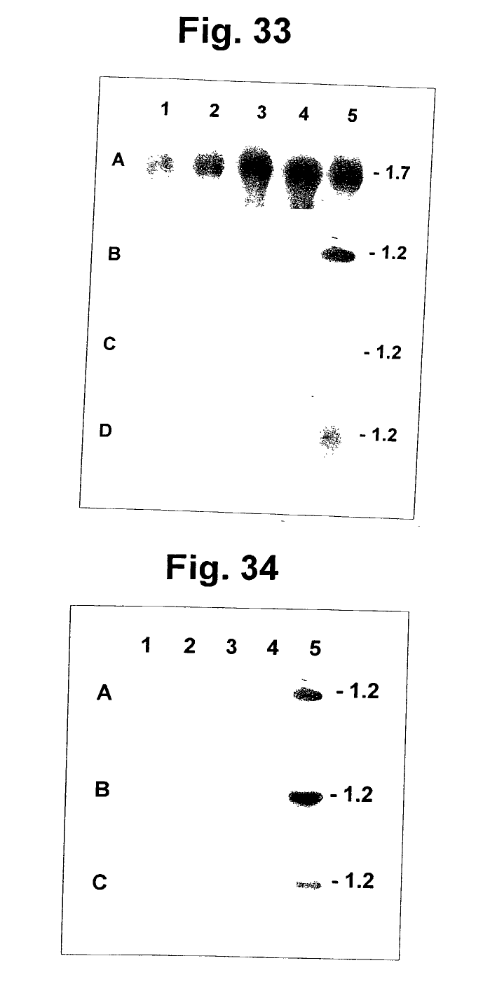

For proof of the inducibility of the AXDH gene by the corresponding sugar, the A. adeninivorans LS3 yeast cells were first cultivated for 48 hours in a YMM with 2% glucose as C source, before it was transferred into the medium with 2% xylit, 2% galactose or 5% sorbite. After the shift to xylit or sorbite-containing media, there is accumulation of AXDH transcript in the yeast cells. With the help of Northern hybridizations, AXDH transcripts can be detected 19 hours after the shift. In contrast with this, in the case of a shift from glucose to galactose medium, 19 hours is not sufficient to detect AXDH transcripts (FIG. 33).

In the last step, the influence of high xylit concentrations on the AXDH transcript accumulation of A. adeninivorans LS3 was tested. For this purpose, the yeast cells were firstly cultivated in YMM with 2% glucose for 48 h, before they were transferred to a new YMM with 2%, 5%, and 23% xylit. Independent of the xylit concentration, there is AXDH transcript accumulation in the yeast cells after 19 hours of cultivation. The highest AXDH transcript concentrations were reached when YMM with 5% xylit was used (FIG. 34).

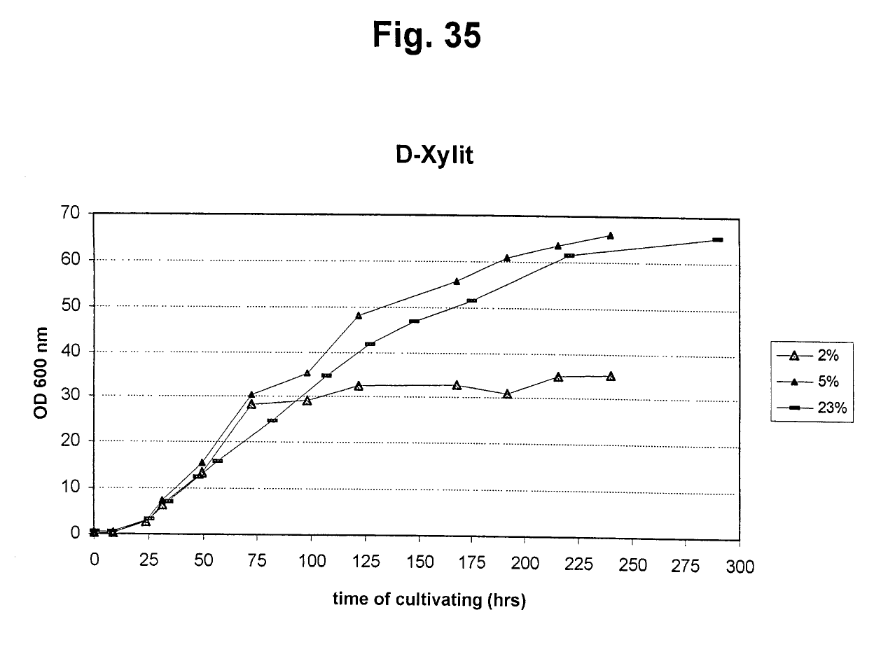

To determine whether A. adeninivorans LS3 can grow even in the presence of high concentrations of xylit, the yeast was cultivated in the presence of 2%, 5% and 23% xylit and the growth course was monitored via determination of the optical density. With an optical density of (at 600 nm) approx. 70 in the stationary growth phase, 2-fold higher cell densities were reached when 5% and 23% xylit were used as C source in comparison with 2% xylit. Thus, the maximum reachable cell titre can be considerably increased (FIG. 35) by rising the xylit concentration >2%.

### Example 16

### Construction of Expression Vectors for Synthesis of Protein in Arxula

The plasmid pUC 19 (Invitrogen, USA) was used as starting plasmid for the construction of expression vectors, used for the production of proteins in a yeast of the Arxula genus. Different elements were introduced in several cloning steps, suitable for the selection of the transformed host organisms with the expression vectors, for maintaining the inserted nucleic acids in the host organism and for expression of the coding gene for the desired protein.

1. Selection Marker

Different selection markers were used for selection of Arxula transformants, which contain at least one expression vector. On the one hand, the hph gene from E. coli was used as selection marker, which enables selection of transformants on the basis of its hygromycin B resistance (Rösel and Kunze 1995; Rösel and Kunze 1998). To ensure correct transcription of the hph gene in Arxula, the hph gene was placed under the control of the constitutively expressed TEF1 promoter of Arxula adeninivorans (Rösel and Kunze 1995) and the PHO5 terminator of Saccharomyces cerevisiae (Arima et al. 1983) was used as terminator.

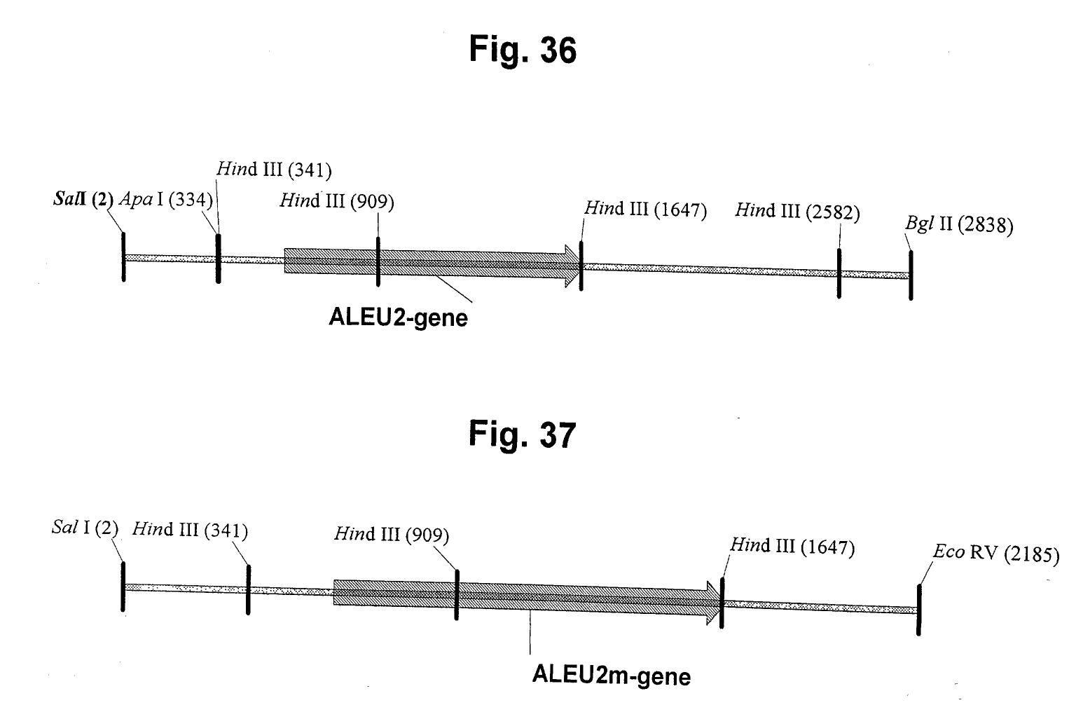

Alternatively, the ALEU2 gene from A. adeninivorans LS3 was used as selection marker. For isolation of this gene, S. cerevisiae SEY6210 [leu2] (Robinson et al. 1988) was transformed with a plasmid gene bank with cDNA of A. adeninivorans LS3 (Yep 112-cDNA) and the transformants obtained were tested after complementing the leu2 mutation. Of the LEU2 prototrophic transformants obtained, the plasmid DNA was isolated and analysed after retransformation in E. coli. All analysed cDNA areas of these screened plasmids were identical. A 2838 bp long Sa/l/Bg/ll fragment with the complete ALEU2 gene (FIG. 36) was incorporated between the Sa/l and the Bg/ll interfaces of the plasmid pUC19 (Plasmid pBSALEUSALI). The nucleotide sequence fo the ALEU2 gene of A. adeninivorans (SEQ ID NO: 15) shows a 78.6%ige homology to the LEU2 gene of S. cerevisiae.

For better handling of the ALEU2 gene, the nucleotide sequence was modified. Via mutagenesis, the ApaIresctriction site was changed so that there was no longer a recognition site for this restriction enzme. The modified ALEU2 gene, is called ALEU2m gene hereafter. At the same time, the fragment with the ALEU2m gene was shortened to 2187 bp and flanked with the SalI and EcoRV restriction site with the help of the PCR method (FIG. 37; SEQ ID NO: 16).

2. Integration-/Replication Sequences for Arxula

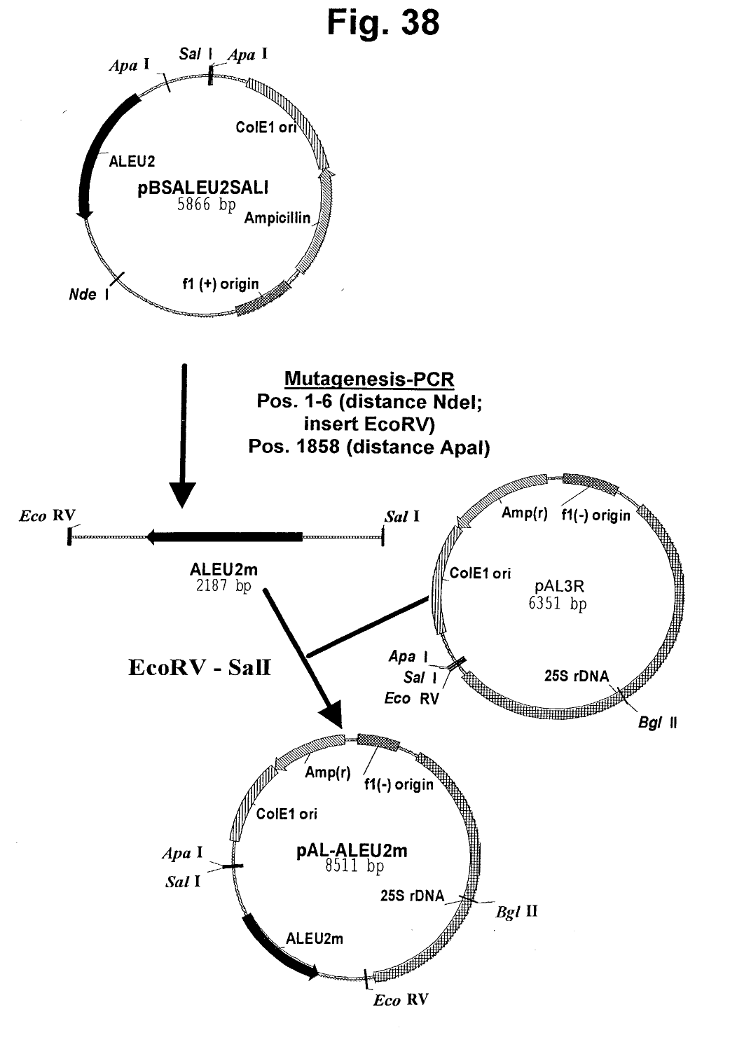

To make available expression vectors that are integrated into the genome of a yeast of the Arxula genus, an approximately 3.4 kbp long EcoRI fragment of the 25S-r DNA of Arxula adeninivorans (Rösel and Kunze 1998; Rösel and Kunze 1996) was inserted into the EcoRI interface of the pUC19 plasmid (plasmid pAL3R). Then, the EcoRV/SalI DNA fragment was inserted into the 25S-rDNA containing the plasmid PAL3R (FIG. 38) together with the ALEU2m gene. The resulting plasmid pAL-ALEU2m can be transformed in A. adeninivorans according to the method described by Rösel and Kunze (1998) after linearization with BglII. For this purpose, the strain A. adeninivorans G1211 [ALEU2] was used. The transformants obtained can be selected by ALEU2m, gene via complementing of the ALEU2 mutation.

The transformants obtained were characterised. To determine the copy number of the plasmids integrated into the genome, Southern hybridization was used. For this purpose, the chromosomal DNA was isolated from A. adeninivorans G1121/pAL-ALEU 2m, constrained with BglII, separated with gel electrophoresis, blotted on nitrocellulose and hybridized against the radioactively labelled pUC 19 plasmid or a Bl/ll fragment with the ALEU2m gene.

In contrast with non-transformed initial strain G1211, the DNA of the transformants G1211/pAL-ALEU2m hybridizes with the plasmid pUC19, which is the E. coli component of the Arxula vector pAL-ALEU2m. The radioactively labelled band has a length of approximately 8.5 kbp, i.e. it is identical with the initial plasmid pAL-ALEU2m. Thus, the plasmid pAL-ALEU2m is integrated into the Bg/ll location of the 25S-rDNA of A. adeninivorans.

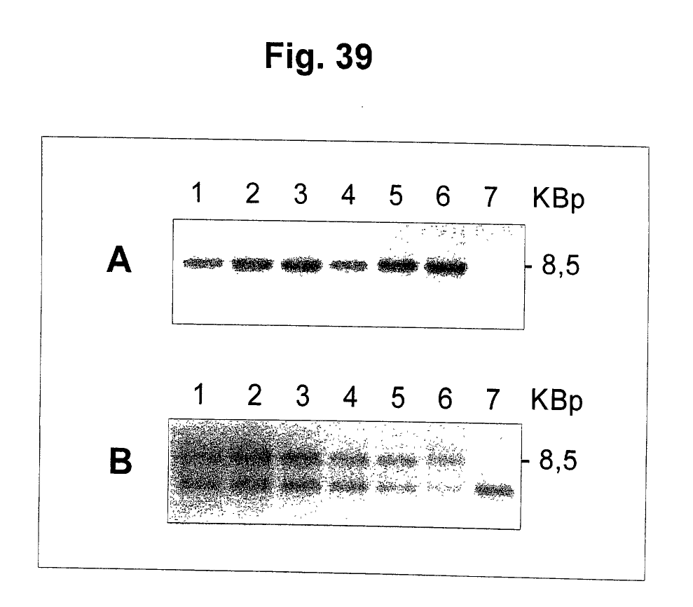

For determination of the copy number of the plasmid integrated into the genome, the Bg/lll-DNA fragment with the ALEU2m gene was used as hybridization probe. This fragment hybridises with the ALEU2 gene of Arxula and with the ALEU2m gene of the transformants. In all samples (initial strain 1211) and transformants), an approximately 3 kbp long fragment could be identified, which represents the Arxula-own ALEU2 gene (FIG. 39). To contrast to this, the transformant hybridises another 8.5 kbp band in the case of chromosomal DNA, which comprises the ALEU2m gene located on the plasmid pAL. If the intensities of the respective ALEU2 bands are compared with the ALEU2m bands, these are identical. Thus, for all tested transformants a copy of plasmid pAL-ALEU2m was integrated into the Arxula genome.

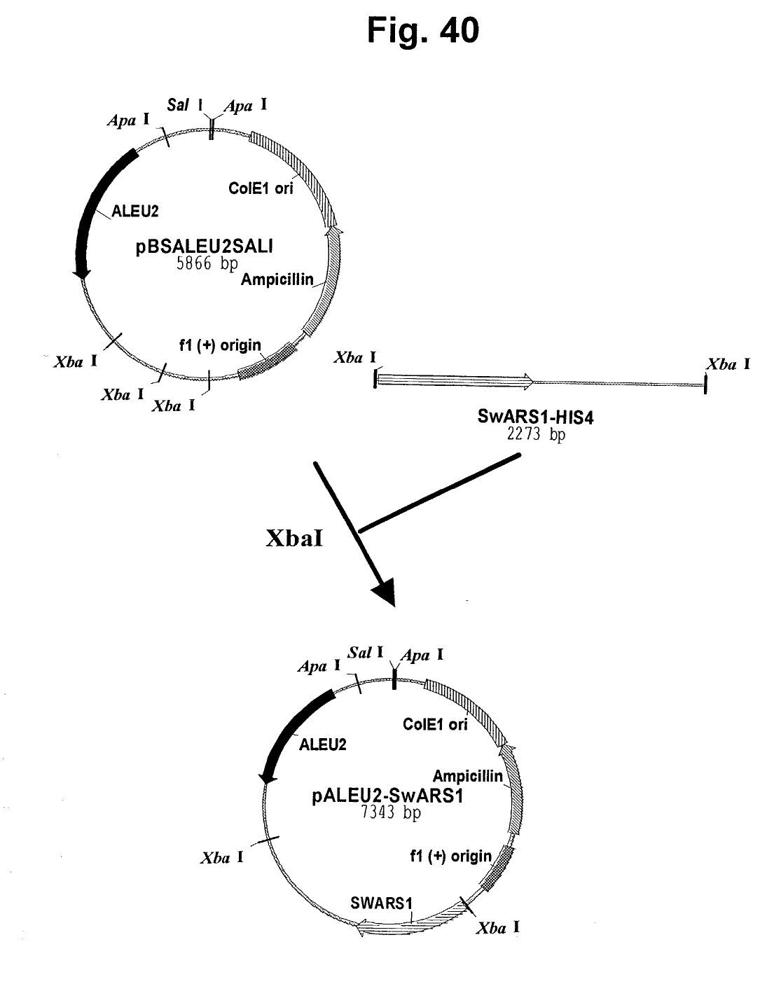

For producing expression plasmids, which can be replicated in a yeast of the genus Arxula, especially A. adeninivorans, the SwARS 1 sequence from Schwanniomyces occidentalis (EMBL-Datenbank, Nr. AJ278886) was tested for its functional ability in the Arxula system. For this purpose, the plasmid pBS-ALEU2-SwARS was constructed according to the method represented in FIG. 40 and transformed in A. adeninivorans G1211 [ALEU2]. The transformants were selected via their complementation of the ALEU2 mutation by the ALEU2 gene and then characterized. For construction of the corresponding plasmid pBS-ALEU2m-SwARS, which contains the modified ALEU2m gene in the place of the ALEU2 gene, the plasmid pBS-ALEU2-SwaRS was cleaved with the restriction enzymes EcoRI and SalI. Then, the EcoRI/SalI fragment obtained was bound to the pAL-ALEU2m (see FIG. 38) from the cleavage of the plasmid with the fragment resulting from the same restriction enzymes with ALEU2m, gene via ligation.

No significant increase in the transformation frequency compared to integrative transformation was found with plasmids pBS-ALEU2-SwARS and pBS-ALEU2m-SwARS. According to the procedure described by Rösel and Kunze (1998) (without prior linearization of the plasmid DNA), 50 to 200 transformants were obtained per μg of DNA. The plasmid DNA was isolated from these transformants and used for the transformation of E. coli. In contrast to integrative transformation, the ARS-containing plasmids could be isolated from the Arxula transformants and re-transformed into E. coli. The plasmid DNA was isolated again from these and constrained. The restriction samples obtained were identical to the initial plasmid (pBS-ALEU2-SwARS1 and. pBS-ALEU2m-SwARS1) Parallel, investigations on the mitotic stability of plasmid in A. adeninivorans G1211 were carried out. For this purpose, transformants were cultivated for approximately 100 generations under selective and non-selective conditions and then plated on selective and non-selective media. Under these conditions, mitotic stabilities of approx. 90% are achieved.

3. Expression Cassette

The expression vectors for producing proteins in Arxula contain an expression cassette with at least one of promoters made available by the invention and a terminator, for example the PH05 terminator from S. cerevisiae. There is a multiple cloning site between the respective promoter and the PH05 terminator (with unical interfaces for EcoRI-BamHI-NotI), with which the DNA fragments can be directly inserted with the genes foreseen for expression directly between the promoter and the PH05 terminator.

### Example 17

### Heterologous Gene Expression with the Help of the AXDH Promoter

1. Expression of the GFP Gene

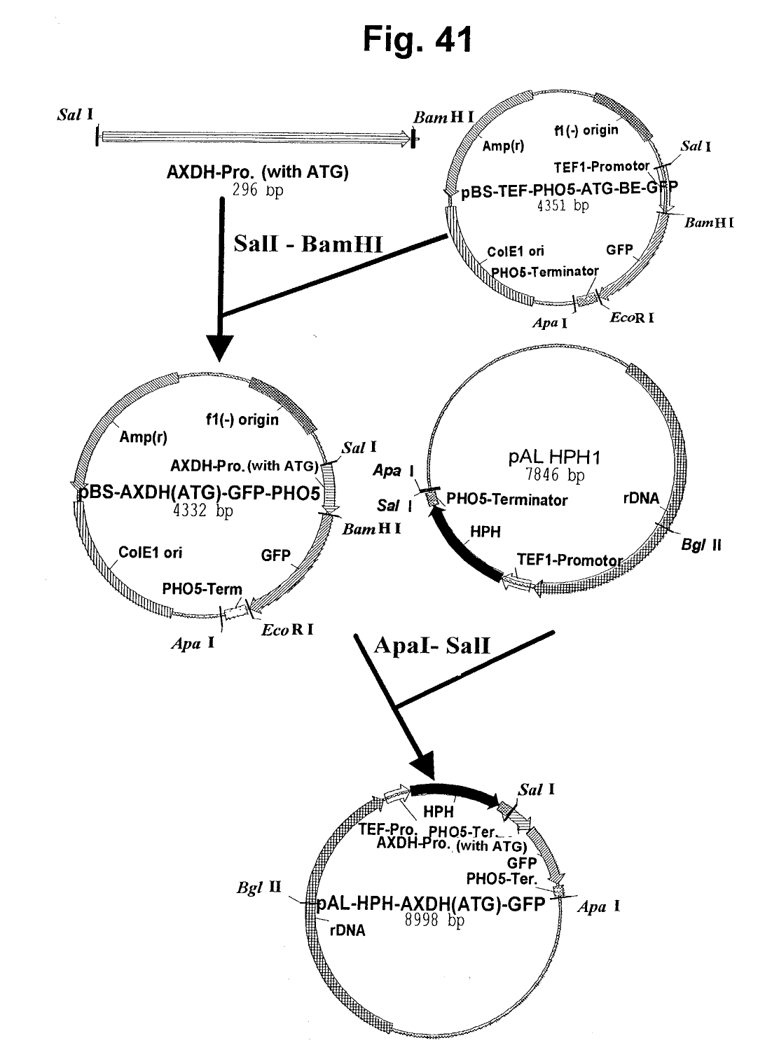

For the expression of the GFP gene (Kahana and SIlver 1996), the AXDH promoter including the start codon of the AXDH gene was used. For this purpose, the promoter region including 281 nucleotides (SEQ ID NO: 18) including start codon ATG was flanked with the restriction recognition sites for SalI and BamHI. The amplication product obtained via PCR, can then be inserted “in frame” before the coding area of the GFP gene. The cassette obtained with AXDH promoter -GFP gene -PHO5 terminator could be cleaved with SalI/ApaI from the plasmid pBS-AXDH(ATG)-GFP-PHO5 and inserted into the Arxula plasmid pAL-HPH 1 (Rösel and Kunze 1998). The resulting plasmid pAL-HPH-AXDH (ATG)-GFP (FIG. 41) can be directly transformed in A. adeninivorans LS3 and 135 after linearising with BglII. The transformants obtained were selected via their hygromycin B resistance.

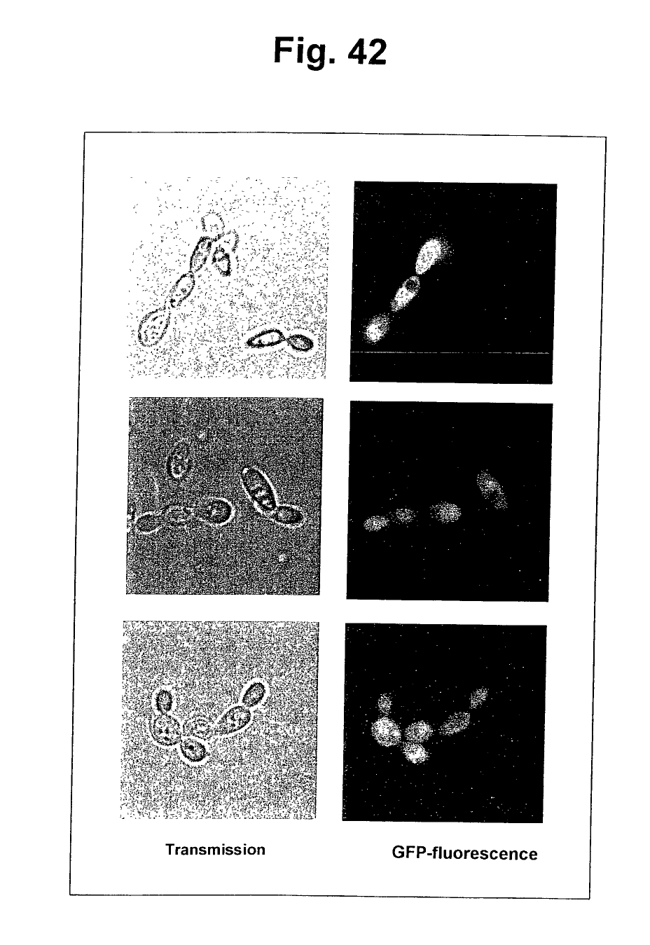

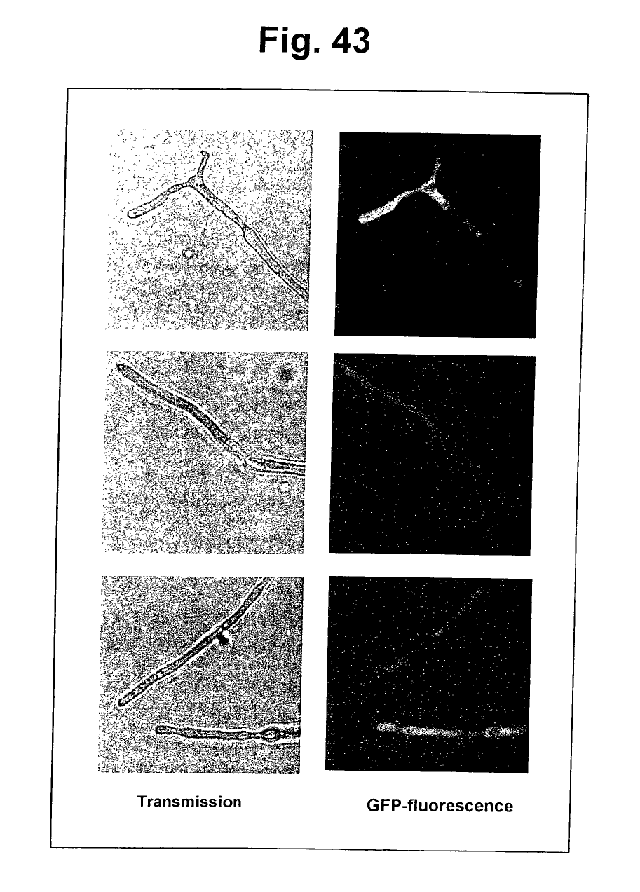

Information about the GFP accumulation was provided with the help of fluorescence microscopy. For this purpose, the transformants A. adeninivorans LS3/pAL-HPH-AXDH (ATG)-GFP and 135/pAL-HPH-AXDH (ATG)-GFP were cultivated for 48 h in YMM (Tanaka et al. 1967) with 2% xylit, harvested and the cells were directly analysed with the fluorescence microscope. Since the GFP gene has no signal sequence, the gene product (GFP) should be located in the cytoplasm, confirmed by the fluorescence investigations performed (FIG. 42, 43). The initial strains A. adeninivorans LS3 and 135 carried along as control exhibited no florescence.

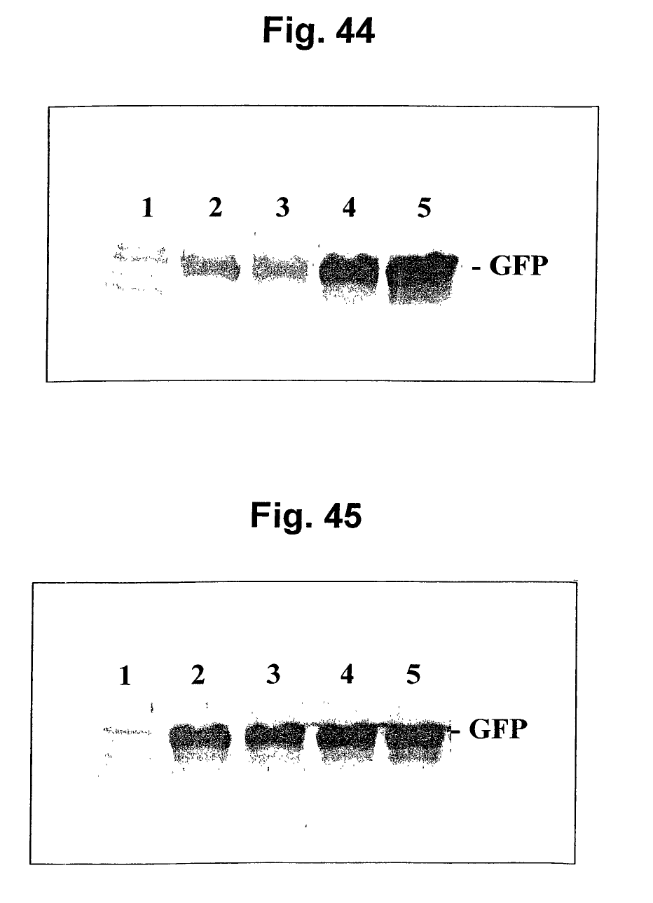

At the same time, GFP was detected by Western-Blot. For this purpose, the transformants A. adeninivorans LS3/pAL-HPH-AXDH(ATG)-GFP and A. adeninivorans 135/pAL-HPH-AXDH(ATG)-GFP were cultivated for 48 h in YMM with 2% xylit, harvested with the yeast cells or mycelium mechanically decomposed. The protein extracts obtained could be used directly for Western blot analysis. A polyclonal anti-GFP antibody (Molecular Probes, USA) was used as antibody (FIGS. 44, 45).

Parallel performed cultivations of the Arxula transformants in YMM with 2% glucose as C source proved again that the AXDH promoter is a promoter that can be regulated. For example, when such a YMM was used, it was not possible to detect GFP with fluorescence microscopy nor with Western Blot analysis.

2. Expression of the HSA Gene

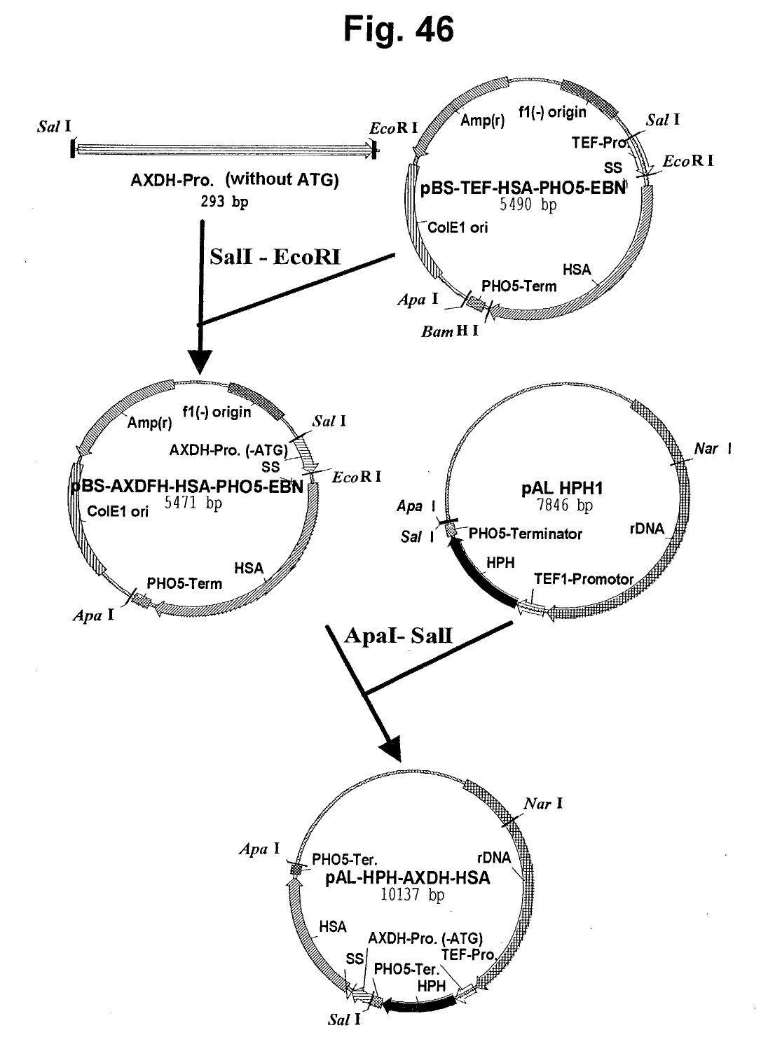

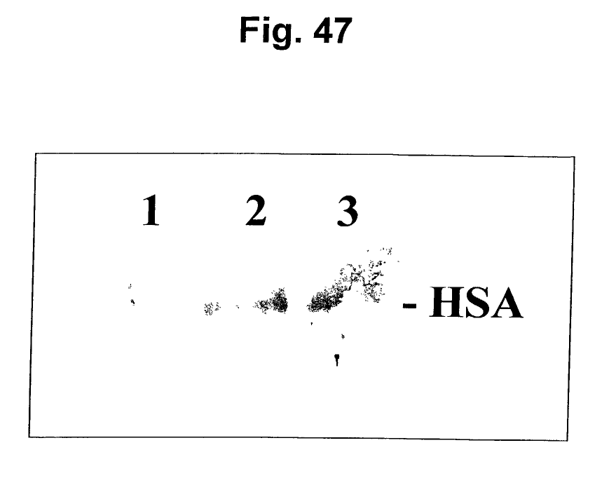

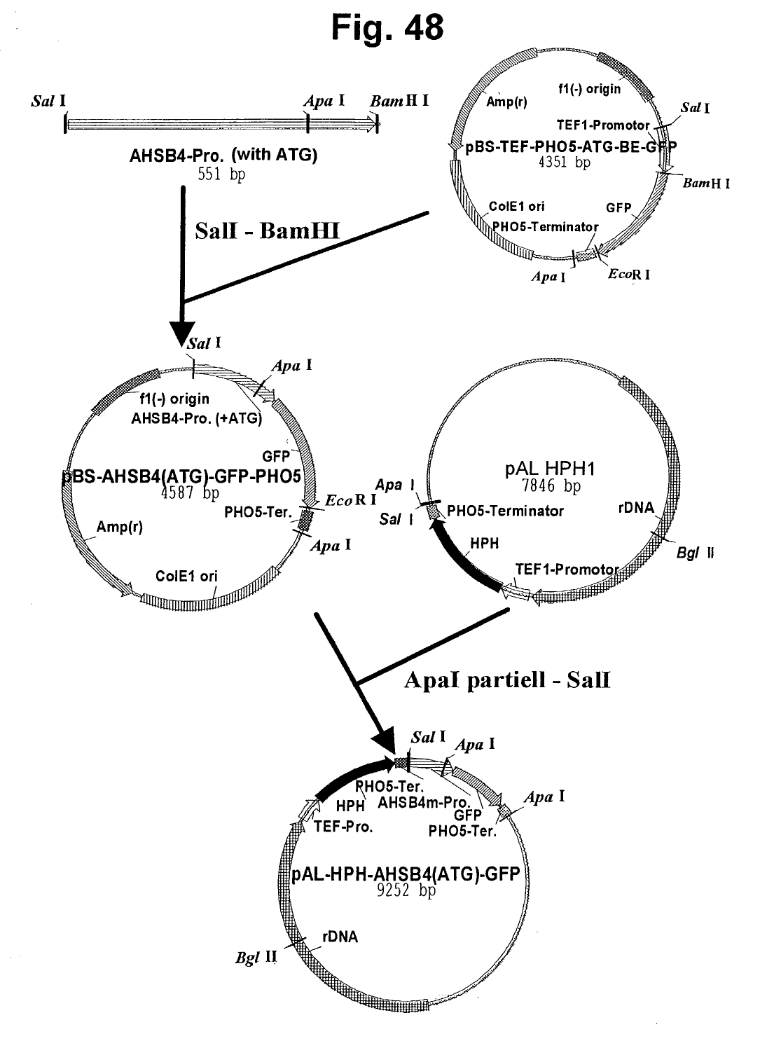

The AXDH promoter (without start codon from AXDH gene) was used for expression of the HSA gene. For this purpose, the promoter region comprising of 281 nucleotides (SEQ ID NO: 18) was flanked with the restriction recognition sites for SalI and EcoRI. The amplification product obtained via the PCR could directly inserted before the HSA gene. The cassette obtained with AXDH promoter—HSA—Gen—PHO5-terminator could be cleaved from the plasmid pBS-AXDH-HSA-PHO5-EBN with Sa/l-ApaI and inserted into the Arxula plasmid pAL-HPH1 (FIG. 46). The plasmid pAL-HPH-AXDH-HSA could be directly transformed into A. adeninivorans LS3 and 135 after linearising with Narl. The transformants obtained were selected via their hygromycin B resistance.

With the use of anti-HSA antibodies, the HSA expression was detected in the Western Blot analysis. For this purpose, the transformants A. adeninivorans LS3/pAL-HPH-AXDH-HSA and 135/pAL-HPH-AXDH-HSA were cultivated for 48 h in YMM (Tanaka et al. 1967) with 2% xylit, harvested and the cells as well as cultivation medium were used directly for Western Blot analysis. Since the HSA gene has its own signal sequence, which transports this protein from the human cell, it should be detectable in the cultivation medium. Experiments showed that more than 90% of the total HAS from the Arxula transformants were secreted into the cultivation medium. In the initial strains A. adeninivorans LS3 and 135 used as control, neither intra nor extracellular HSA could be detected.

### Example 18

### Heterologous Gene Expression With the Help of the AHSB Promoter

1. Expression of the GFP Gene

The expression of the GFP gene is possible with the help of the AHSB4 promoter. For this purpose, the promoter region (SEQ ID NO: 17) comprises of 536 nucleotides including start codon ATG was flanked with the restriction recognition sites for SalI and BamHI. The amplification product obtained via the PCR must subsequently be inserted “in frame” before the GFP gene. The cassette obtained with the AHSB4 promoter-GFP gene-PHO5-terminator could be cleaved from of the plasmid pBS-AHSB4(ATG)-GFP-PHO5 with SalI-ApaIand inserted into the plasmid pAL-HPH1 (FIG. 48). After linearising with Bg/ll the plasmid pAL-HPH-AHSB4(ATG)-GFP could be directly transformed into A. adeninivorans LS3 and 135. The transformants obtained were selected via their hygromycin B resistance.

For testing the GFP accumulation, fluorescence microscopy was used. For this purpose the transformants A. adeninivorans LS3 /pAL-HPH-AHSB4(ATG)-GFP and 135/pAL-HPH-AHSB4(ATG)-GFP was cultivated for 48 h in YMM (Tanaka et al. 1967) with 2% glucose, harvested and the cells analysed in fluorescence microscope.

2. Expression of the HSA Gene

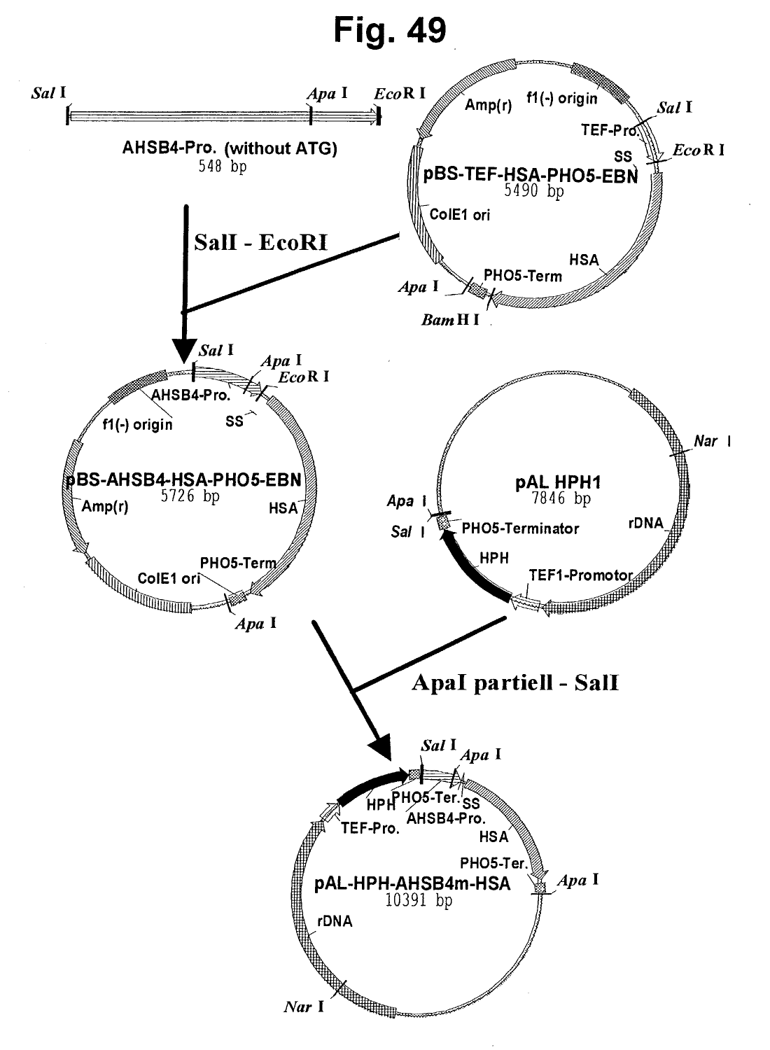

With the help of the AHSB4 promoter, the HSA gene in A. adeninivorans could also be expressed. For this purpose, the promoter region comprising of 536 nucleotides (SEQ ID NO: 17) was flanked with the restriction recognition sites for Sa/I and EcoRI. The amplification product obtained via the PCR can be directly inserted before the HSA gene. The cassette obtained with the AHSB4-promoter-HSA-gene-PHO5-terminator could be cleaved from the plasmid pBS-AHSB4-HSA-PHO5 with SalI-ApaI and inserted into the Arxula plasmid pAL-HPH1 (FIG. 49). After linearisation with NarI, the plasmid pAL-HPH-AHSB4-HSA could be directly transformed in A. adeninivorans LS3 and 135. The transformants obtained were selected via their hygromycin B resistance.

### Example 19

### Heterologous Gene Expression of lacZ with the Help of the GAA Promoter

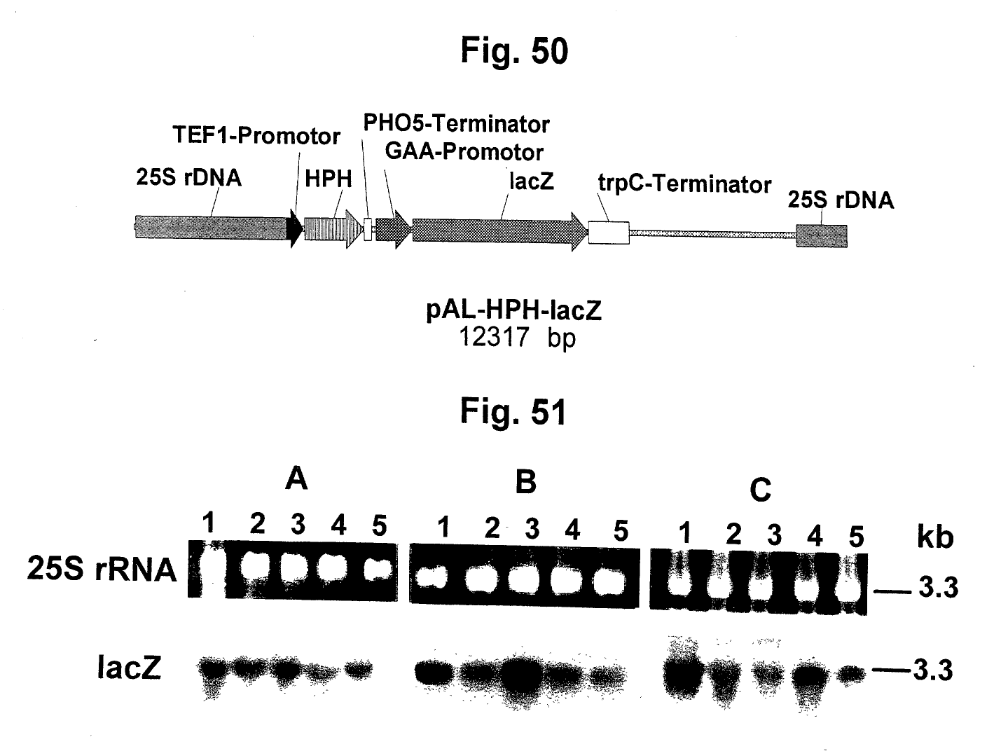

The GAA promoter was used for expression of the lacZ gene of E. coli. For this purpose a cassette with the GAA promoter (A. adeninivorans)-lacZ-Gen (E. coli)-PHO5-terminator (S. cerevisiae) was constructed and inserted into the Arxula vector pAL-HPH1 (FIG. 50). After linearisation of the plasmid obtained pAL-HPH-lacZ) by cleaving with BglIl, it was integrated into the 25S-rDNA of the Arxula genome by transformation and the transformants obtained were characterised.

In contrast to the initial strain A. adeninivorans 135, the transformant 135/pAL-HPH-lacZ has an approximately 2.1 kbp size lacZ transcript. However, this expression was only achievable when YMM was used with maltose or starch as C source. If glucose is used for the experiments, no lacZ transcript can be detected. Thus, the expression of the GAA promoter-lacZ-gene-cassette takes place as a function of the C source used and the culture (yeast cell-mycelium) (FIG. 51).

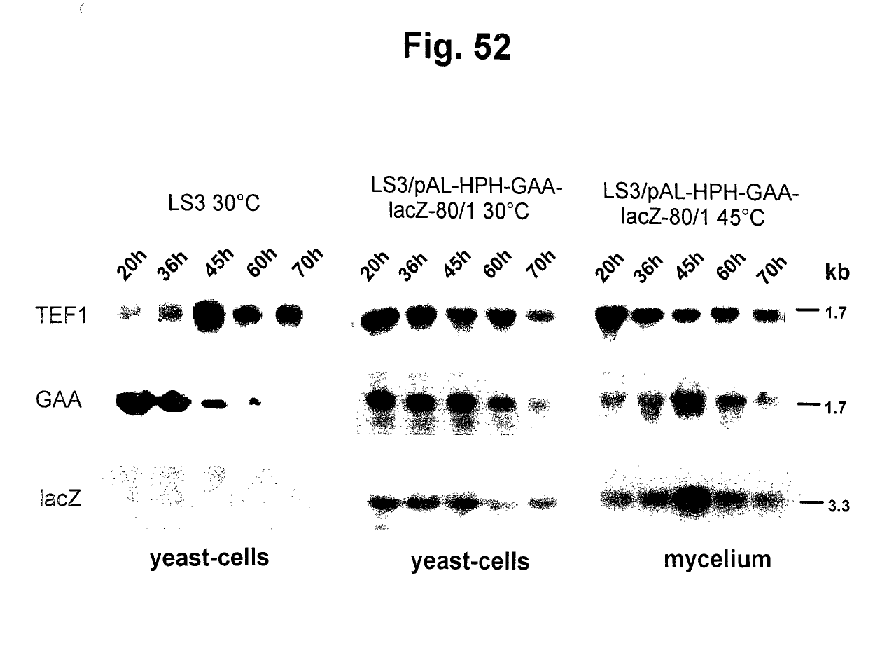

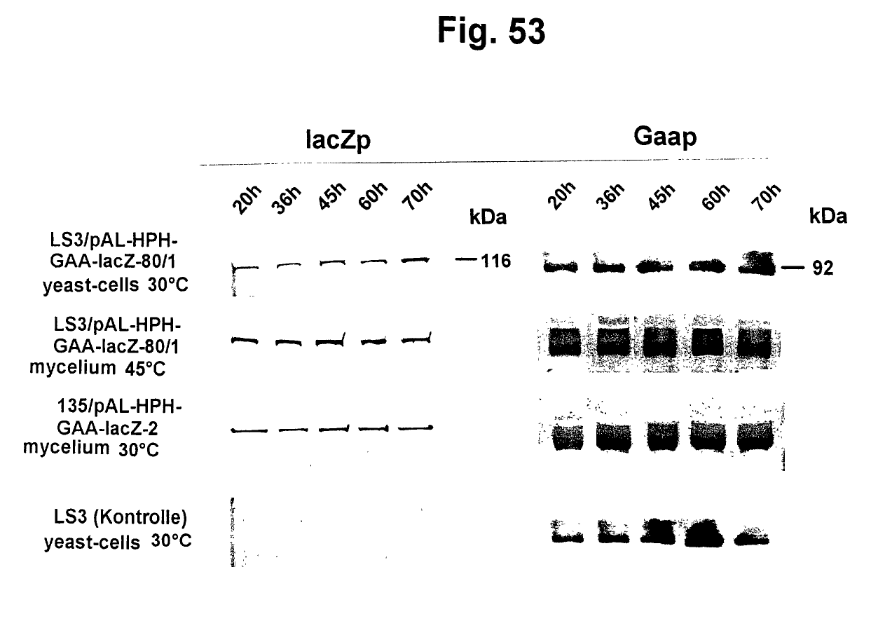

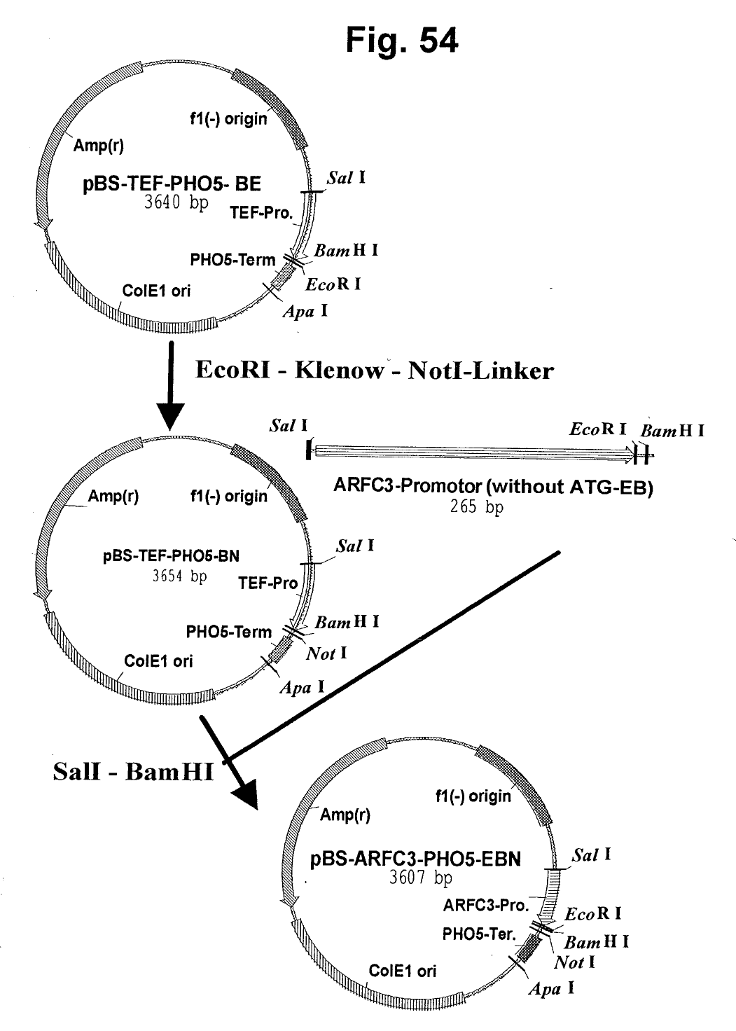

The TEF I-, GAA- and lacZ transcript concentrations in A. adeninivorans LS3, cultivated at 30° C. (yeast cells) and LS3/pAL-HPH-GAA-lacZ-80/1, cultivated at 30° C. (yeast cells) or 45° C. (myceliums) were determined by Northern hybridisation. For this purpose, cultures were cultivated in YMM with maltose as C source. The cells were harvested after 20, 36, 45, 60 and 70 hours of cultivation. The total RNA was isolated and used for Northern hybridisations. A 0.9 Kb Hindlll/Bg/ll fragment with the TEFI gene or a 3 kbp BamHI/EcoRI-DNA fragment with the lacZ gene was used as radioactive labeled probe (FIG. 52). The intracellular 13-galactosidase (lacZp) and the extracellular glucoamylase (Gaap) from A. adeninivorans LS3/pAL-HPH-GAA-lacZ-80/1, cultivated at 30° C. (yeast cells) or 45° C. (mycelium), 135/pAL-HPH-GAA-lacZ-2 cultivated at 30° C. (mycelium) and LS3, cultivated at 30° C. (yeast cells), were identified by Western blot analysis. An anti-Gaap antibody and an anti-B-galactosidase antibody (Boehringer-Mannheim, BRD) were used as an antibody (FIG. 53).

### Example 20

### Heterologous Gene Expression with the Help of the ARFC3 Promoter

For the expression of the HSA gene in A. adeninivorans under the control of the ARFC3 promoter, pAL-HPH1 was used as Arxula basis plasmid, which contains the hph gene derived from E. coli as selection marker. With this plasmid, genetically labelled and non-labelled strains can be transformed and the transformants obtained can be selected via their hygromycin B resistance.

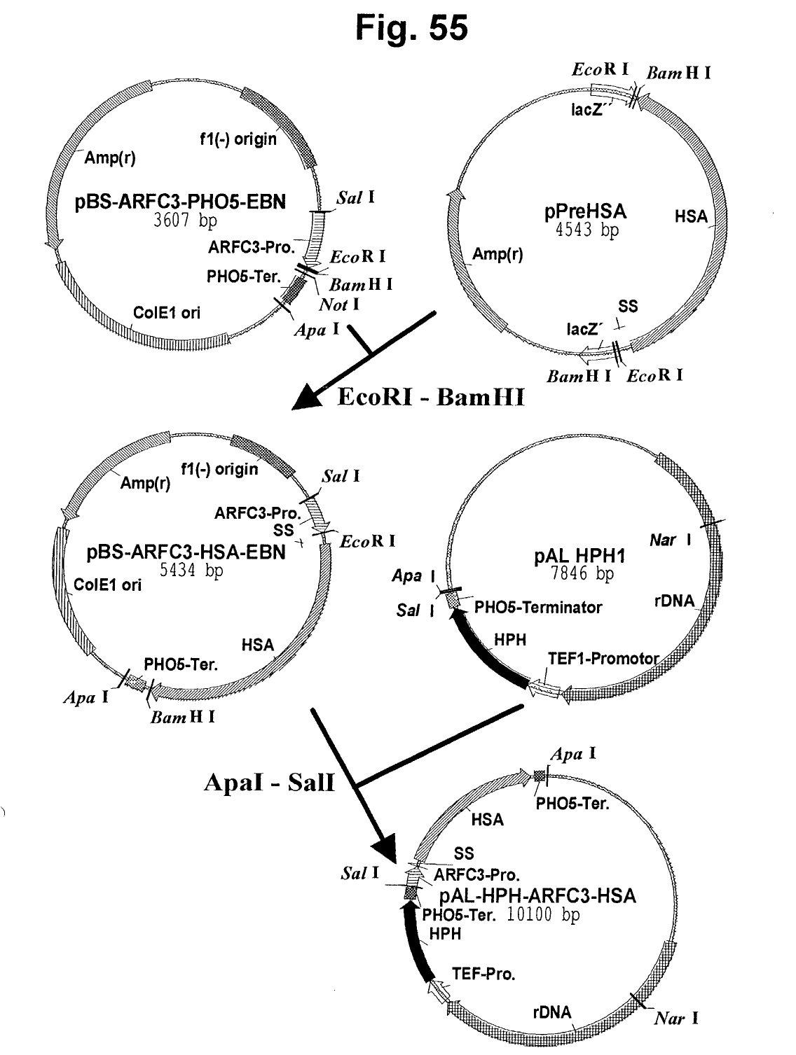

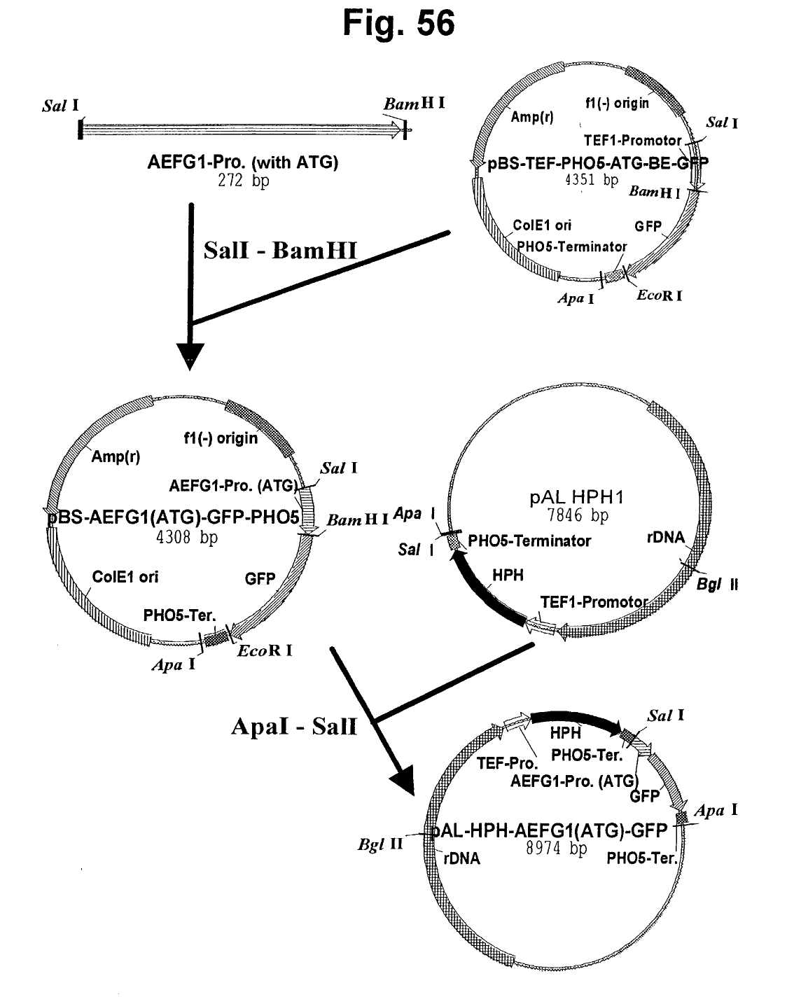

The HSA gene was inserted directly between the ARFC3 promoter from A. adeninivorans and the PHO5 -terminator of S. cerevisiae. The plasmid pAL-HPH-ARFC3-HSA (FIGS. 54 and 55) obtained after another cloning step was transformed in A. adeninivorans after linearization with the restriction enzyme Narl (Ehel). The wild type strain A. adeninivorans LS3 and a mutant with altered dimorphism (produces mycelium already at 30° C =A. adeninivorans 135) were used as host.

After characterisation of the Arxula transformants by proof of integration of the hph gene and HAS gene in their chromosomal DNA, the expression was analysed.

### Example 21

### Heterologous Gene Expression with the Help of the AEFG1 Promoter

1. Expression of the GFP Gene

The GFP gene could also be expressed with the help of the AEFG1 promoter. For this purpose, a promoter region comprising of 257 nucleotides (SEQ ID NO: 19) including start codon ATG was flanked with the restriction recognition sites for SalI and BamHI. The amplification product obtained via the PCR had to be inserted “in frame” before the GFP gene. The cassette with the AEFG1-promoter-GFP-gene-PHO5-terminator obtained could be cleaved from the plasmid pBS-AEFG1(ATG)-GFP-PHO5 with Sa/l-ApaI and inserted into the Arxula-plasmid pAL-HPH1. The plasmid pAL-HPH-AEFG1(ATG)-GFP can be directly transformed in A. adeninivorans LS3 and 135 after linearisation with Bg/ll. The transformants obtained were selected via their hygromycin B resistance.

The GFP accumulation was tested with the help of fluroscence microscopy. For this purpose the transformants A. adeninivorans LS3/pAL-HPH-AEFG1 (ATG)-GFP and 135/pAL-HPH-AEFG1(ATG)-GFP were cultivated for 48 h in YMM (Tanaka et al. 1967) with 2% glucose cultivated, harvested and the cells analysed with the fluorescence microscope. In this example as well the GTP protein should be in the cytoplasm. The fluorescence investigations performed confirm an expression of the GFP gene. However, it was relatively low. The shift from aerobic to anaerobic conditions led to an increase in the GFP accumulation. In contrast to this, the initial strains used as control exhibited no fluorescence.

2. Expression des HSA-Gens

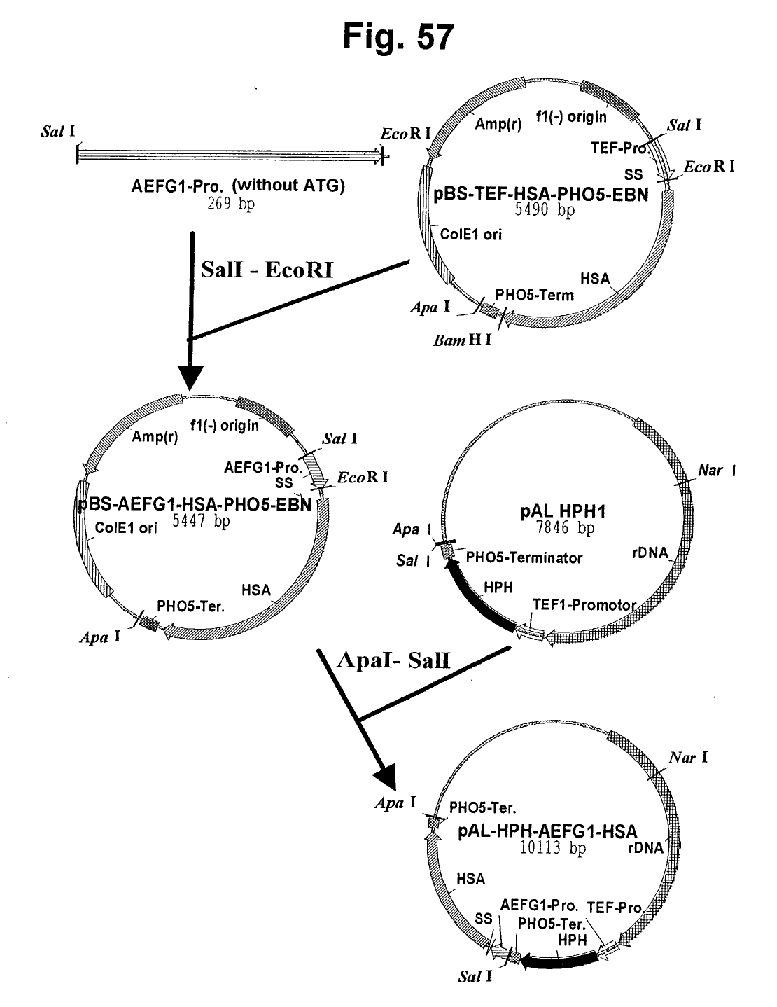

With the help of the AEFG1 promoter, the HSA− gene in A. adeninivorans could also be expressed. For this purpose, the promoter region comprising of 257 nucleotides (SEQ ID NO: 19) was flanked with the restriction recognition sites for SalI and EcoRI. The amplification product obtained via the PCR can be inserted directly before the HAS gene. The cassette with the AEFG1 promoter—HSA-gene—PHO5-terminator obtained could be cleaced back from the plasmid pBS-AEFG1-HSA-PHO5 with SalI-ApaI and inserted into the Arxula-plasmid pAL-HPH1 (FIG. 57). The plasmid pAL-HPH-AEFG1-HSA could be directly transformed in A. adeninivorans LS3 and 135 after linearisation with Narl. The transformants obtained were selected via their hygromycin B resistance.

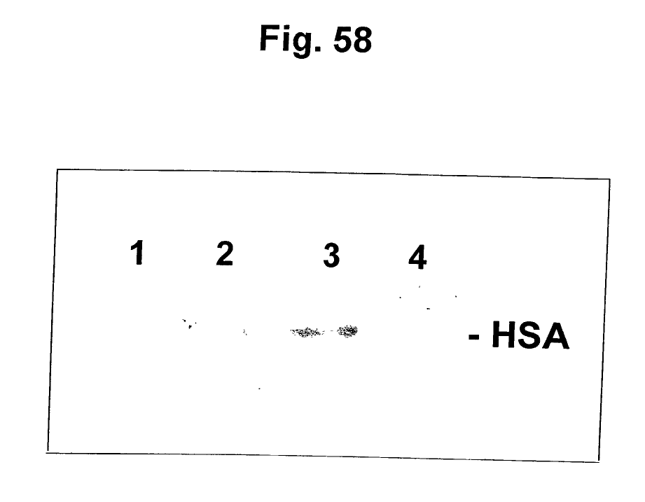

With the use of anti-HSA antibodies (Biotrend Chemikalien, BRD), the HSA expression was proven in Western blot analysis. For this purpose, the transformants A. adeninivorans LS3/pAL-HPH-AEFG1-HSA and 135/pAL-HPH-AEFG1-HSA were cultivated in YMM for 48 h (Tanaka et al. 1967) with 2%glucose, harvested and the cells as well as cultivation medium were used directly for Western blot analysis. Since the HAS gene contains its own signal sequence, which transports this protein out of the human cells, HAS should have been detectable in the cultivation medium. The experiments showed that more than 90% of the entire HSA from the Arxula transformants was secreted into the cultivation medium. In the initial strains A. adeninivorans LS3 and 135, used as control neither extracellular nor intracellular HSA could be detected (FIG. 58). The shift from aerobic to anaerobic conditions led to an increase in the HSA accumulation.

It will be appreciated by those skilled in the art that changes could be made to the embodiments described above without departing from the broad inventive concept thereof It is understood, therefore, that this invention is not limited to the particular embodiments disclosed, but it is intended to cover modifications within the spirit and scope of the present invention as defined by the appended claims.

### References

Altschul S F. et al.(1990) Basic local alignment search tool. J. Mol. Biol. 215: 403-410.

Arima K, Oshima T, Kubotal, Nakamura N, Mizunaga T, Tohe A (1983) The nucleotide sequence of the yeast PHO5 gene: a putative precursor of repressible acid Phosphatase contains a signal peptide. Nucleic Acids Res 11: 1657-1672

Devereux, J. et al. (1984) Nucleic Acids Research 12 (12): 387.

Gellissen G, Hollenberg, C P (1997) Application of yeasts in gene expression studies: a comparison of Saccharomyces cerevisiae, Hansenula polymorpha and Kuyveromyces lactis—a review. Gene 190, 87-97.

Hollenberg C P, Gellissen G (1997) Production of recombinant proteins by methylotrophic yeasts. Curr Opinion Biotechnol 8, 554-560.

Johnston M, Hillier L, Riles L, Albermann K, Andre B, Ansorge W, Benes V, Bruckner M, Delius H, Dubois E, Dusterhoft A, Entian K D, Floeth M, Goeffeau A, Hebling U, Heumann K, Heuss-Neitzel D, Hilbert H, Hilger F, Kleine K, Kotter P, Louis E, Messenguy F, Mewes H W, Hoheisel J D (1997) The nucleotide sequence of Saccharomyces cerevisiae chromosome. Nature 387, 87-90.

Kahana J A, Silver P A (1996) Use of A. victoria green fluorescent protein to study protein dynamics in vivo. Current protocols in molecular biology: 9.6.13.-9.6.19. John Wiley, New York.

Kuchler K, R. Steme, J Thomer (1989) Saccharomyces cerevisiae STE6 gene product: a novel pathway for protein export in eukaryotic cells. EMBO J 8, 3973-3984.

Kunze G, Bode R, Schmidt H, Samsonova I A, Birnbaum D (1987) Identification of a lys2 mutant of Candida maltosa by means of transformation. Curr Genet 11: 385-391

A. Kunze G, Kunze I. (1994) Characterization of A. adeninivorans strains from different habitats. Antonie van Leeuwenhoek 65: 607-614.

Kunze G, Kunze I. (1996) Arxula adeninivorans. In: Wolf K (Hrsg.) Nonconventional yeasts. Springer Verlag, Berlin-Heidelberg-New York, S. 389-409.

Kurihara T, Ueda M, Kanayama N, Kondo J, Teranishi Y, Tanaka A (1992) Peroxisomal acetoacetyl-CoA thiolase of an n-alkane-utilizing yeast Candida tropicalis. Eur J Biochem 210, 999-1005.

Middelhoven W J, Hoogkamer-Te Niet M C, Kreger van Rij N J W (1984) Trichosporon adeninovorans sp. nov., a yeast species utilizing adenine, xanthine, uric acids, putrescine and primary n-alkylamines as the sole source of carbon, nitrogen and energy. Antonie van Leeuwenhoek 50, 369-378.

Needleman und Wunsch (1970) J. Mol. Biol 48: 443-453.

Nishi K, M Yoshida, M Nishimura, M Nishikawa, M Nishiyama, S Horinouchi, T Beppu (1992) A leptomycin resistance gene of Schizosaccharomyces pombe encodes a protein similar to the mammalian P-glycoproteins. Mol Microbiol 6, 761-769.

Ramaswamy N T, Li L, Khalil M, Cannon J F. (1998) Regulation of yeast glycogen metabolism and sporulation by Glc7p protein phosphatase. Genetics 149(1):57-72.

Robinson J S, Klionsky D J, Banta L M, Emr S D (1988) Protein sorting in Saccharomyces cerevisiae. Isolation of mutants defective in the delivery and processing of multiple vacuolar hydrolases. Mol Cell Biol 8: 4936-4948.

Rose M D, Winston F, Hieter P. (1990) Methods in yeast genetics. A laboratory manual. Cold Spring Harbor Laboratory. Cold Spring Harbor. N.Y.

Rösel H, Kunze G (1995) Cloning and characterization of a TEF gene for elongation factor 1α from the yeast Arxula adeninivorans. Curr Genet. 28, 360-366.

Rösel H und Kunze G (1996) Identification of a group-I intron within the 25S rDNA from the yeast Arxula adeninivorans. Yeast 12: 1201-1208.

Rösel H, Kunze G (1998) Integrative transformation of the dimorphic yeast Arxula adeninivorans LS3 based on hygromycin B resistance. Curr Genet 33, 157-163.

B. Sambrook J, Fritsch E F, Maniatis T. (1989) Molecular cloning: a laboratory manual. 2. Ausgabe, Cold Spring Harbor Laboratory, Cold Spring Harbor, N.Y.

Samsonova I A, Kunze G, Bode R, Böttcher F(1996) A set of genetic markers for the chromosomes of the imperfect yeast A. adeninivorans. Yeast 12: 1209-1217.

Stoltenburg R, Wartmann T, Kunze I, Kunze G. (1995). Reliable method to separate free and membrane-bound polysomes from different species. Bio/Techn 18: 564-568.

Sudbery P (1996) The expression of recombinant proteins in yeasts. Curr Opin Biotechnol 7, 517-524.

Tanaka A, Ohnishi N, Fukui S (1967) Studies on the formation of vitamins and their function in hydrocarbon fermentation. Production of vitamin B6 by Candida albicans in hydrocarbon medium. J Ferment Technol 45, 617-623.

Van der Walt J P, Smith M T, Yamada Y (1990) Arxula gen.nov. (Candidaceae), a new anamorphic, arthroconidial yeast genus. Antonie van Leeuwenhoek 57, 59-61.

Wartmann T, Erdmann J, Kunze I, Kunze G. (2000) Morphology-related effects on gene expression and protein accumulation of the yeast Arxula adeninivorans LS3. Arch. Microbiol. 173: 253-261

Wartmann T, Rösel H, Kunze I, Bode R, Kunze G. (1998) AILV1 gene from the yeast Arxula adeninivorans LS3—a new selective transformation maker. Yeast 14: 1017-1025

What is claimed is:

1. Method for synthesising one or several proteins in a host cell of the yeast genus Arxula, comprising of:
(i) cloning at least a nucleic acid, which encodes a heterologus protein, in a an expression vector, which contains an inducible promoter from a yeast of the genus Arxula or a promoter from a constitutive expressed gene, selected from the ARFC3 gene and the AHSB4 gene, from a yeast of the genus Arxula, so that the cloned nucleic acid is under the transcriptional control of the promoter;
(ii) inserting the expression vector obtained in (i) into a suitable host cell of the yeast genus Arxula for synthesising the heterologous protein;
(iii) cultivating of the host cell obtained in (ii) and, if necessary
(iv) inducing the promoter;
(v) harvesting of the protein with a known method.

2. Method according to claim 1, characterized in that the ARFC3 promoter derived from the ARFC3 gene of a yeast of the genus Arxula comprises the sequence given in SEQ ID NO: 1 or SEQ ID NO: 2.

3. Method according to claim 1, characterised in that the AHSB4 promoter derived from the AHSB4 gene of a yeast of the genus ARXULA comprises the sequence given in SEQ ID NO: 17.

4. Method according to claim 1, characterised in that the inducible promoter is derived from a gene of the yeast genus Arxula, whose expression is associated with a change in cell morphology.

5. Method according to claim 4, characterised in that the promoter is derived from the GAA gene, AACP10 gene, the APCR17 gene or the APRE4 gene of a yeast of the genus Arxula.

6. Methods according to claim 4 or 5, characterised in that the promoter comprises the sequence given in SEQ ID NO: 3, SEQ ID NO: 4, SEQ ID NO: 5 or SEQ ID NO: 6.

7. Method according to claim 1, characterised in that the inducible promoter is inducible by changing the salt concentration in the culture medium.

8. Method according to claim 7, characterised in that the promoter is derived from the AACE1 gene, ATAL1 gene or the AGLC7 gene of the Arxula yeast genus.

9. Method according to claim 7 or 8, characterised in that the promoter comprises the sequence given in SEQ ID NO: 7, SEQ ID NO: 8 oder SEQ ID NO: 9.

10. Method according to claim 1, characterised in that the inducible promoter is inducible by a change in salt and/or Fe(II) ion concentration in the culture medium.

11. Method according to claim 10, characterised in that the promoter is derived from the AFET3 gene of the AFTR1 gene of the Arxula yeast genus.

12. Method according to claim 10 or 11, characterised in that the promoter comprises the sequence given in SEQ ID NO: 10 or SEQ ID NO: 11.

13. Method according to claim 1, characterised in that the inducible promoter is inducible by chaing the salt and/or oxygen concentration in the culture medium.

14. Method according to claim 13, characterised in that the promoter is derived from the AEFG1 gene of an Arxula yeast genus.

15. Method according to claim 13 or 14, characterised in that the promoter comprises the sequence given in SEQ ID NO: 12.

16. Method according to claim 1, characterised in that the inducible promoter can be induced by a toxic agent.

17. Method according claim 16, characterised in that the promoter is derived from the ASTE6 gene of the yeast genus Arxula.

18. Method according to claim 16 or 17, characterised in that the promtoer comprises the sequence given in SEQ ID NO: 13.

19. Method according to claim 16, characterised in that the promoter is inducible by lyptomycin B.

20. Method according to claim 19, characterised in that the promoter is derived from the APMD1 gene of the Arxula yeast genus.

21. Method according to claim 19 or 20, characterised in that the promoter comprises the sequence given in SEQ ID NO: 14.

22. Method according to claim 1, characterised in that the inducible promoter can be induced with xylit, xylose, sorbite and/or galactose.

23. Method according to claim 22, characterised in that the promoter is derived from the AXDH gene of the Arxula yeast genus.

24. Method according to claim 22 or 23, characterised in that the promoter comprises the sequence given in SEQ ID NO: 18.

25. Method according to one of the previous claims, characterised in that the promoter is derived from a gene of the Arxula adeninivorans yeast.

26. Method according to one of the preceding claims, characterised in that the expression vector still contains at least one marker gene.

27. Method according to claim 26, characterised in that the marker gene comprises the ALEU2 gene from Arxula adeninivorans or a variant of it.

28. Method according to claim 27, characterised in that the marker gene comprises the sequence given in SEQ ID NO: 15 or SEQ ID NO: 16.

29. Method according to one of the preceding claims, characterised in that the expression vector comprises a sequence, which makes possible the integration of the vector or a part of it into the genome of the host cell.

30. Method according to one of claims 1 to 28, characterised in that the expression vector comprises a sequence, which makes possible the replication of the vector in the host cell.

31. Nucleic acid molecule, comprising of:
(1) an inducible promoter of a gene from the yeast genus Arxula, whose expression is associated with a change in cell morphology, and selected from the following nucleic acids:
a nucleic acid with the sequence given in SEQ ID NO: 4;
a nucleic acid with the sequence given in SEQ ID NO: 5;
a nucleic acid with the sequence given in SEQ ID NO: 6;
a nucleic acid with a sequence that is at least 50% identical with one of the sequences given in (a), (b) or (c);
a nucleic acid that hybridises with the complementary strand of one of the a nucleic acids given in (a), (b) or (c);
a derivative of one of the nucleic acids given in (a), (b) or (c) obtained by substitution, addition and/or deletion of one or several nucleotides;
a fragment of one of the nucleic acids given in (a) to (f), which retains the function of the inducible promoter;
a combination of several of the nucleic acids given in (a) to (g), whereby the sequences of the nucleic acids can be identifical or different; or
(2) a nucleic acid with a sequence, complementary to the sequence of one of the the nucleic acids given in (a) to (h).

32. Nucleic acid molecule, comprising of:
(1) an inducible promoter by changing the salt concentration in the culture medium, selected from the following nucleic acids:
a nucleic acid with the sequence given in SEQ ID NO: 7;
a nucleic acid with the sequence given in SEQ ID NO: 8;
a nucleic acid with the sequence given in SEQ ID NO: 9;
(a) a nucleic acid with a sequence that is at least 50% identical with one of the sequences given in (b) or (c);
(b) a nucleic acid that hybridises with the complementary strand of one of the nucleic acids given in (a), (b), or (c);
(c) a derivative of one of the nucleic acids given in (a), (b), or (c) obtained by substitution, addition and/or deletion of one or several nucleotides;
(d) a fragment of one of the nucleic acids given in (a) to (f), which retains the function of the inducible promoter;
(e) a combination of several of the nucleic acids given in (a) to (g), whereby the sequences of the nucleic acids can be identical or difference; or
(2) a nucleic acid with a sequence, that is complementary to the sequence of one of the nucleic acids given in (a) to (h).

33. Nucleic acid molecule according to claim 31 or 32, characterised in that the nucleic acid given in (d) is at least 70% identical with one of the seqwuences given in (a), (b) or (c) or their complementary sequence.

34. Nucleic acid molecule according to claim 31 or 32, characterised in that the nucleic acid given in (d) is at least 90% identical with one of the sequences given in (a), (b), or (c) or their complementary sequence.

35. Nucleic acid molecule according to claim 31 or 32, characterised in that the nucleic acid given in (d) is at least 95% identical with one of the sequences given in (a), (b) or (c) or their complementary sequence.

36. Nucleic acid molecule, comprising of:
(1) an promoter which is inducible by changing the salt and/or Fe(ll) ion concentration in culture medium, selected from the following nucleic acids:
(a) a nucleic acid with the sequence given in SEQ ID NO: 10;
(b) a nucleic acid with the sequence given in SEQ ID NO: 11;
(c) a nucleic acid with a sequence having at least 50% identity with one of the sequences given in (a) or (b);
(d) a nucleic acid that hybridises with the complementary strand of one of the nucleic acids given in (a) or (b);
(e) a derivative of one of the nucleic acids given in (a) or (b) obtained by substitution, addition and/or deletion of one or several nucleotides;
(f) a fragment of one of the nucleic acids given in (a) to (e), which retains the function of the inducible promoter;
(g) a combination of several of the nucleic acids given in (a) to (f), whereby the sequences of the nucleic acids can be identical or different; or
(2) a nucleic acid with a sequence that is complementary to the sequence of one of the nucleic acids given in (a) to (g).

37. Nucleic acid molecule, comprising of:
A promoter inducible by an toxic agent, selected from the following nucleic acids:
a nucleic acid with the sequence given in SEQ ID NO: 13;
a nucleic acid with the sequence given in SEQ ID NO: 14;
a nucleic acid with a sequence having at least 50% identity with one of the sequences given in (a) or (b);
a nucleic acid that hybridises with the complementary strand of the nucleic acid given in (a);
a derivative of the nucleic acid given in (a) obtained by substitution, addition, and/or deletion of one or several nucletides;
a fragment of one of the nucleic acids given in (a) to (e) that retains the function of the inducible promoter;
a combination of several of the nucleic acids given in (a) to (g), whereby the sequences of the nucleic acids can be identifical or different; or
(2) a nucleic acid with a sequence that is complementary to the sequence of one of the nucleic acids given in (a) to (g).

38. Nucleic acid molecule according to claim 36 or 37, characterised in that the nucleic acid given in (c) is at least 70% identical with one of the sequences given in (a) or (b) or their complementary sequence.

39. Nucleic acid molecule according to claim 36 or 37, characterised in that the nucleic acid given in (c) is at least 90% identical with one of the sequences given in (a) or (b) or their complementary sequence.

40. Nucleic acid molecule according to claim 36 or 37, characterised in that the nucleic acid given in (c) is at least 95% identical with one of the sequences given in (a) or (b) of their complementary sequence.

41. Nucleic acid, comprising of:
an inducible promoter by changing the salt and/or oxygen concentration in the culture medium, selected from the following nucleic acids:
a nucleic acid with the sequence given in SEQ ID NO: 12;
a nucleic acid with a sequence having at least 50% identity with the sequence given in (a);
a nucleic acid that hybridises with the complementary strand of the nucleic acid given in (a);
a derivative of the nucleic acid given in (a) obtained by substitution, addition and/or deletion of one or several nucleotides;
a fragment of one of the nucleic acids given in (a) to (d) that retains the function of the inducible promoter;
a combination of several of the nucleic acids given in (a) to (e), whereby the sequences of the nucleic acids can be identical or different; or
(2) a nucleic acid with a sequence that is complementary to one of the nucleic acids given in (a) to (f).

42. Nucleic acid molecule, comprising of:
a promoter inducible by xylit, xylose, sorbite and/or galactose, selected from the following nucleic acids:
(a) a nucleic acid with the sequence given in SEQ ID NO: 18;
(b) a nucleic acid with a sequence having at leasat 50% identity with the sequence given in (a);
a nucleic acid that hybridises with the complementary strand of the nucleic acid given in (a);
a derivative of the nucleic acid given in (a) obtained by substitution, addition and/or deletion of one or several nucleotides;
a fragment of one of the nucleic acids given in (a) to (d) that retains the function of the inducible promoter;
a combination of several of the nucleic acids given in (a) to (e), whereby the sequences of the nucleic acids can be identifical or different; or
(2) a nucleic acid with a sequence that is complementary to the sequence of one of the nucleic acids given in (a) to (f).

43. Nucleic acid molecule comprising of:
(1) a constitutive promoter selected from the following nucleic acids:
(a) a nucleic acid with the sequence given in SEQ ID NO: 17;
(b) a nucleic acid with a sequence having at least 50% identity with the sequence given in (a);
(c) a nucleic acid that hybridises with the complementary strand of the nucleic acid given in (a);
(d) a derivative of the nucleic acid given in (a) obtained by substitution, addition and/or deletion of one or several nucleotides;
(e) a fragment of one of the nucleic acids given in (a) to (d) that retains the function of a constitutive promoter;
(f) a combination of several of the nucleic acids given in (a) to (e), whereby the sequences of the nucleic acids can be identical or different; or
(2) a nucleic acid with a squence that is complementary to the sequence of the nucleic acids given in (a) to (f).

44. Nucleic acid molecule according to one of claims 41 to 43, characterised in that the nucleic acid given in (b) is at least 70% identical with the sequence given in (a) or its complementary sequence.

45. Nucleic acid molecule according to one of claims 41 to 43, characterised in that the nucleic acid given in (b) is at least 95% identical with the sequence given in (a) or its complementary sequence.

46. Nucleic acid molecule according to one of claims 41 to 43, characterised in that the nucleic acid given in (b) is at least 95% identical with one of the sequence given in (a) or its complementary sequence.

47. Nucleic acid molecule according one of claims 31 to 46, characterised in that it comprises at least one nucleic acid sequence for a heterologous gene that is under the transcriptional control of the promoter.

48. An expression vector that comprises at least a nucleic acid molecule according to one of claims 31 to 47.

49. An expression vector according to claim 48, characterised in that it contains at least one marker gene.

50. An expression vector according to claim 49, characterised in that the marker gene is the ALEU2 gene from Arxula adeninivorans or a variant thereof.

51. An expression vector according to claim 50, characterised in that the marker gene comprises a sequence according to SEW ID NO: 15 or SEQ ID NO: 16.

52. An expression vector according to one of claims 48 to 51, characterised in that it comprises a sequence that makes possible the integration of the vector of part of it into the genome of a host cell of the yeast genus Arxula.

53. An expression vector according to one of claims 48 to 51, characterised in that it comprises a sequence that makes possible the replication of the vector in the host cell of the yeast genus Arxula.

54. A host cell containing a nucleic acid molecule according to one of claims 31 to 47 or an expression vector according to one of claims 48 to 53.

55. A host cell according to claim 54, characterized by the fact that it belongs to the yeast genus Arxula.

56. A host cell according to claim 55, characterized by the fact that it is Arxula adeninivorans.

57. Kit comprising of:
an expression vector according to one of claims 48 to 53, suitable for cloning of a nucleic acid, which encodes a heterologous protein, and a host cell suitable for induction of the promoter and synthesising the heterologous protein.

58. Use of a nucleic acid molecule according to one of claims 31 to 47 or an expression vector according to one of claims 48 to 53 or a host cell according to one of claims 54 to 56 or a kit according to claim 57 for expression of a gene under the control of the promoter.

59. Use of a nucleic acid molecule according to one of claims 31 to 47 or an expression vector according to one of claims 48 to 53 or a host cell according to one of claims 54 to 56 or a kit according to claim 57 for synthesising one protein or several proteins.
